(19) United States
(12) Patent Application Publication
(10) Pub. No.: US 2023/0280342 A1
(43) Pub. Date: Sep. 7, 2023

(54) IDENTIFICATION OF LOW-DENSITY INFLAMMATORY NEUTROPHILS IN SEVERE COVID-19 PATIENTS

(71) Applicant: UNIVERSITY OF LOUISVILLE RESEARCH FOUNDATION, INC., Louisville, KY (US)

(72) Inventors: Jun YAN, Louisville, KY (US); Jiapeng HUANG, Louisville, KY (US); Samantha MORRISSEY, Louisville, KY (US)

(73) Assignee: UNIVERSITY OF LOUISVILLE RESEARCH FOUNDATION, INC., Louisville, KY (US)

(21) Appl. No.: 18/008,391

(22) PCT Filed: Jun. 4, 2021
(86) PCT No.: PCT/US2021/035988

§ 371 Date: Dec. 5, 2022

Related U.S. Application Data
(60) Provisional application No. 63/035,422, filed on Jun. 5, 2020.

(51) Int. Cl. G01N 33/569

(52) U.S. Cl. CPC G01N 33/56983; G01N 33/56972

(57) ABSTRACT

In certain aspects, methods are provided for treating a subject having been diagnosed with coronavirus disease 2019 (COVID-19) with a therapeutic agent that inhibits low-density inflammatory neutrophil (LDN) population expressing intermediate levels of CD 16 (CD16^{Int}). In certain aspects, methods are provided for treating a subject having been diagnosed with coronavirus disease 2019 (COVID-19) with a therapeutic agent that inhibits CD66b+CD16^{Int}CD11b^{Int}CD44^{low}CD40⁺ low-density inflammatory band (LDIB) neutrophil population. In certain aspects, methods are provided for detecting the seventy level of coronavirus disease 2019 (COVID-19) in a subject, comprising measuring the level of CD16^{Int} low-density inflammatory neutrophil (LDN) in plasma as compared to a control.

### CROSS-REFERENCE TO RELATED APPLICATION

This application claims priority to U.S. Provisional Application No. 63/035,422 that was filed on Jun. 5, 2020. The entire content of the applications referenced above is hereby incorporated by reference herein.

### BACKGROUND

December 2019 saw the emergence of a novel viral pathogen, severe acute respiratory syndrome coronavirus 2 (SARS-CoV-2). At the beginning of June 2020, there were over 6.6 million cases worldwide with close to 400,000 reported deaths. SARS-CoV-2 is considered a lower respirators, tract pathogen that gains access to the body by binding to the angiotensin-converting enzyme 2 (ACE-2) on the surface of alveolar epithelial type II cells. The virus causes a clinical disease called coronavirus disease 2019 (COVID-19). While the majority of persons infected with COVID-19 experience mild to moderate symptoms of pharngitis, rhinorrhea, and low-grade pyrexia, approximately 20% of patients experience a severe influenza-like manifestation of the disease. Clinically, these patients present with bilateral pneumonia progressing to acute respiratory distress syndrome (ARDS) with a marked decreased in pulmonary function requiring mechanical ventilation. The fluid accumulation in the lungs that is pathognomonic for ARDS results from a combination of virally induced lung injury as well as the rapid influx of immune cells to fight the infection. These recruited inflammatory mediators are often in a hyper-activated state causing a phenomenon known as “cytokine storm.” There have been a variety of cytokines associated with cytokine storm including interleukin-6 (IL-6), interleukin-1β (IL-1B), and tumor necrosis factor-α (TNFα). If the high levels of cytokines go unresolved, patients are at an increased risk of vascular hyperpermeability, multi-organ failure, and death. Levels of all three cytokines have been found to be elevated in the peripheral blood of COVID-19 patients.

Severe COVID-19 patients have a distinct immunological phenotype characterized by lymphopenia and neutrophilia. Patients with an increased neutrophil to lymphocyte ratio (NLR) have reported worse clinical outcomes. Lung specimens at autopsy showed a marked infiltration of neutrophils into the lung tissue. Neutrophils are thought to he recruited to the lungs to aid in the clearance of the viral pathogens through phagocytosis, secretion of reactive oxygen species, and cytotoxic granule release. However, prolonged activation of these neutrophils has been linked to adverse outcomes in patients with influenza. Specifically, neutrophil populations in patients with severe H1N1 influenza infection showed increased extracellular net formation, neutrophil mediated alveolar damage, and delayed apoptosis. These factors predominately contributed to mortality in animal models of the disease.

Accordingly, diagnostic markers are needed to assist clinicians to better delineate which patients are at the highest risk for developing thromboembolic complications of COVID-19 and to determine when to treat with appropriate immunomodulatory agents.

### SUMMARY

In certain embodiments, the present invention provides a method of treating coronavirus disease 2019 (COVID-19) in a subject, comprising the step of administering to the subject a therapeutically effective therapeutic agent, wherein the therapeutic agent inhibits CD66b⁺CD16^{Int}CD11b^{Int}CD44^{low}CD40⁺ low-density inflammatory band (LDIB) neutrophil population.

In certain embodiments, the present invention provides a method of treating coronavirus disease 2019 (COVID-19) in a subject, comprising the step of administering to the subject a therapeutically effective therapeutic agent, wherein the therapeutic agent inhibits COVID-19-associated coagulopathy (CAC).

In certain embodiments, the present invention provides a method of treating coronavirus disease 2019 (COVID-19) in a subject, comprising the step of administering to the subject a therapeutically effective therapeutic agent, wherein the subject has a lower level of CD16^{Int}CD44^{Low}l CD11b^{Int} low-density neutrophils, and wherein the therapeutic agent is respiratory therapy.

In certain embodiments, the present invention provides a method of treating a patient having been diagnosed with coronavirus disease 2019 (COVID-19) with a therapeutic agent that inhibits low-density inflammatory neutrophil (LDN) population expressing intermediate levels of CD16 (CD16^{Int}).

In certain embodiments, the present invention provides a method of detecting the severity level of coronavirus disease 2019 (COVID-19) in a patient, comprising measuring the level of CD161^{Int} low-density inflammatory neutrophil (LDN) in plasma as compared to a control.

### BRIEF DESCRIPTION OF DRAWINGS

FIGS. 1A-1D. The identification of a CD16 intermediate low-density neutrophil population in COVID-19 patients. (FIG. 1A) Neutrophil and lymphocyte percentages and the neutrophil to lymphocyte ratio in whole blood as measured by a clinical complete blood count (CBC) in HDs and patients with moderate and severe COVID-19 infection. Data are pooled from serial blood samples collected from 5 HDs, and serially from 6 moderate patients and 7 severe patients starting from the day of enrollment. Each draw from each patient represents one data point and is related to the condition of the patient (moderate or severe) on that day. HD (n=6), Moderate timepoints (n=13), Severe timepoints (n=27). Pie charts depict representative data of the neutrophil to lymphocyte ratio (NLR) in HDs, severe and moderate patients. (FIG. 1B) The percent of CD16 negative (CD16^{Neg)}, CD16 intermediate (CD16^{Int}), and CD16 high (CD16^{High}) neutrophils from whole blood samples among HD (n=5), moderate (n=22), and severe (n=30) serially drawn COVID-19 samples. Samples are gated the CD45⁺CD66b⁺ population and show an increased CD16^{Int} population in moderate and severe COVID-19 patients. Summarized data and representative dot plots are shown. (FIG. 1C) Representative dot plots (left) and summarized data (right) showing the overall percent of CD66b⁺ neutrophils (gated on viable, CD45⁺) as well as CD16^{Neg}, CD16^{Int}, and CD16^{High} subsets as found in Ficoll isolated PBMCs analyzed using CyTOF mass cytometry in healthy donors (n=5), moderate samples (n=21) and severe samples (n=36), (FIG. 1D) Representative viSNE cluster plots generated using CyTOF work flow show the CD45⁺ PBMC populations in HDs, and patients with moderate and severe COVID-19. Plots highlight an increased intensity of the CD66b⁺ neutrophil population (left) and CD16⁺ populations (right) in HDs versus moderate and severe COVID-19 patients. Red circles indicate the location of the neutrophil population while the blue circle indicates the CD16^{Int} population. In all summarized data, the mean with standard deviation is represented. p values were determined using a linear mixed effect model, ns=p≥0.05, *p<0.05, **p<0.01, ****p<0.0001.

FIGS. 2A-2H. Phenotypic characteristics of CD16^{Neg}, CD16^{Int}, and CD16^{High} neutrophil populations. (FIG. 2A) Wright Giemsa staining of CD66b⁺ CD16^{Neg} (left), CD16^{Int} (middle), and. CD16^{High} (right) populations that were enriched using Fluorescence Activated Cell Sorting (FACS) show different stages of neutrophil maturation. (FIG. 2B) The heatmap shows differential expression of CD11b (top) and CD44 (bottom) on CD66b⁺ neutrophils in Ficoll isolated PBMCs analyzed via mass cytometry. Here, 6 HDs, 5 moderate COVID-19 patients and 7 severe COVID-19 patient samples from the first day of study enrollment were used. (FIG. 2C) Using mass cytometry, CD11b expression on CD66b⁺ neutrophils segregated into three distinct populations: CD11b high (CD11b⁺⁺). CD11b intermediate (CD11b⁺) and CD11b low (CD11b⁻). CD11b⁺⁺ cells were found to be CD16^{high} (top), CD11b⁺ cells were found to have intermediate CD16 expression (middle) and CD11b⁻ cells showed low CD16 expression (bottom). (FIG. 2D) viSNE cluster plots generated using CyTOF work flow highlight the expression of CD11b in the CD16^{Int} neutrophil population (indicated by the red circle). An increase in the CD11b⁺ population can be seen in moderate and severe COVID-19 patients as compared to HDs. (FIG. 2E) Using mass cytometry, CD44 expression on CD66b⁺ neutrophils segregated into two distinct populations: CD44 positive (CD44^{high}) and CD44 negative (CD44^{low}). The CD44^{high} population is shown to have high expression of CD16 as shown by the histogram, while the CD44^{low} population is shown to have intermediate expression of CD16. Summarized data includes the first sample acquired from each patient enrolled in the study, and shows that the percent of CD66b⁺CD44^{low} neutrophils is significantly increased in severe patients (n=7) as compared to HDs (n=6) and moderate patients (n=5). Statistics were performed using a one-way ANOVA where *p<0.05 FIG. 2 (F) viSNE cluster plots represent the decreased expression of CD44 in the CD16^{Int} neutrophil compartment in severe COVID-19 patients as compared to moderate patients and HDs, as highlighted by the blue circle. (FIG. 2G) The phagocytic capacity of neutrophils from whole blood in HDs, severe and moderate patients was assessed using a pHrodo™ Green E. Coli BioParticles™ phagocytosis assay. Representative histograms show the relative phagocytic capacity of CD16^{Int} populations in HD (left), moderate (middle), and severe patients (right), and summarized data indicates the percent of phagocytic cells in the CD16^{Int} population. HD (n=1), moderate (n=4) and severe (n=4). (FIG. 2H) Wright Giemsa staining of CD66b⁺ neutrophils showed spontaneous NET formation from CD16^{Int} LDIB neutrophils,

FIGS. 3A-3D. The expression of CD40 on neutrophils and correlation with clinical measures of coagulation (FIG. 3A) Heatmaps showing the overall expression of various surface markers on the CD66b⁺ neutrophil population in HDs (n=5), moderate (n=5) and severe (n=6) patients on their first day of study enrollment. (FIG. 3B) Representative viSNE plats showing increased CD40 expression on the overall CD66b⁺ neutrophil population (indicated by the red. circle) healthy donors, moderate and severe COVID-19 patients (left). Summarized expression of CD40 on the overall neutrophil pool as well as on the CD16^{High} and CD16^{Int} neutrophil subsets in COVID-19 patients (right). Data were pooled from serial patient draws throughout the course of their hospital admission and grouped according to patient status. A linear mixed effect model was used to determine significance. (FIGS. 3C, 3D) D-dimer (n=22) and ferritin n=21) values from serial samples from the severe cohort only were correlated with the percent of CD40⁺CD66b⁺ total neutrophils (FIG. 3C) and the percent of CD40⁺CD16^{Int} neutrophils (FIG. 3D). Marginal Pearson correlations were used to indicate statistical significance in all correlations, where **p<0.01, ****p<0.0001.

FIGS. 4A-4C. Correlation of clinical coagulation indicators with neutrophils and LDIBs (FIG. 4A) For severe and moderate patients, the clinical values of D-dimer, Ferritin, Platelets and LDH were acquired from patient charts, and serial blood draws from patients were grouped based on patient status. These values were recorded approximately every other day during hospital admission and were pooled to generate summarized data D-dimer samples: moderate (n=15), severe (n=23). Ferritin samples: moderate (n=16), severe (n=22). Platelet samples: moderate (n=17), severe (n=33), LDH samples: moderate (n=17), severe (n=18). A linear mixed effect model was used to determine significance. *p<0.05 **p<0.01 (FIG. 4B) The D-dimer (n=38). ferritin (n=38), platelet (n=50) and LDH (n=35) levels for all COVID-19 patient samples in FIG. 3A were correlated with the total neutrophil percentage in the Ficoll isolated PBMCs on the day of that charted measurement. (FIG. 4C) The D-dimer, ferritin, platelet and LDH values (n=same above) were correlated with the corresponding percent of CD16^{Int} neutrophils in the Ficoll isolated PBMCs found on the same day at the clinical reading. For all correlation data, a line of best fit is shown to visually examine correlation, with a green line representing a statistically significant correlation, a red line representing a non-significant correlation and an orange line representing a trending correlation that was not significant. Marginal Pearson correlations were used to indicate statistical significance in all correlations, where ns=p≥0.05, **p<0.01, ***p<0.001.

FIGS. 5A-5F. Cytokine production by LDIBs drives clinical features of coagulation (FIG. 5A) An ELISA was used to detect plasma concentrations of IL-6 and TNF-α in each patient sample, HD (n=6), moderate (n=21), severe (n=36) and a mixed linear effect model was used to determine significance between groups. IL-6 and TNF-α levels were then correlated with both the total neutrophil count and the percent of CD16^{Int} neutrophils in the corresponding sample as measured by mass cytometry. Samples that fell below the level of detection of the TNF-α ELISA were excluded from correlation data. (IL-6 n=57, TNF-α n=38) (FIG. 5B) Representative plots of TNF-α (top) and IL-6 (bottom) production from LPS stimulated CD16^{High} and CD16^{Int} neutrophils cultured from whole blood samples of moderate (n=4) and severe patients (n=2), with accompanying summarized data. p values were determined using a student's t-test. (FIG. 5C) Pie charts show the relative contribution of neutrophils to the total TNF-α and IL-6 ex vivo pool as compared to all other immune cells in healthy donors and COVID-19 patients, indicating an increase in the ratio of TNF-α and IL-6 being made by neutrophils in COVID-19 patients. (FIG. 5D) IL-6 plasma concentrations measured in A were also correlated with the clinically measured D-dimer levels from the same day that the sample was acquired (n=38), Ferritin (n=38), Platelets (n=50), and LDH (n=35) (FIG. 5E) TNF-α plasma concentrations measured in A were also correlated with the clinically measured values from the same day that the sample was acquired. Samples that fell below the level of detection of the TNF-α ELISA were excluded from correlation data. D-dimer (n=28), Ferritin (n=27), Platelets (n=41), and LDH (n=23) (FIG. 5F) Serum concentrations of IL-6 and TNF-α were also correlated with one other (n=38). Patient mortality was correlated with plasma TNF-α and IL-6 concentrations using the mean TNF-α and IL-6 level from a patient's samples. Patient mortality was indicated in a binary variable where 1 indicated mortality and 0 was used for non-mortality. For all correlation data, a line of best fit is shown to visually examine correlation, with a green line representing a statistically significant correlation, a red line representing a non-significant correlation and an orange line representing a trending correlation that was not significant. Marginal Pearson correlation were used to indicate statistical significance in all correlations, where ns=p≥0.05, *p<0.05, **p<0.01 ***, p<0.001, **** p<0.0001.

FIG. 6. Pulmonary intravascular coagulopathy (PIC).

FIG. 7. Mass cytometry antibody panel.

FIGS. 8A-8B. Cluster analysis of CD45+ PBMCs in healthy donors, moderate and severe COVID-19 patients. (FIG. 8A) Representative cluster maps for moderate and severe COVID-19 patients as compared to healthy donors. The data was generated from CyTOF based analysis of CD45+ PBMCs isolated from peripheral blood. (FIG. 8B) Heatmap of differential expression pattern of lineage and surface markers in PBMCs of moderate and severe COVID-19 patients as compared to healthy donors. The color key identifies the cluster populations shown above. Here, 5 HDs, 5 moderate COVID-19 patients and 6 severe COVID-19 patient samples from the first day of study enrollment were used to generate the plots.

FIGS. 9A-9B. Longitudinal immune profiling of moderate and severe COVID-19 patients. (FIG. 9A) Representative viSNE plots generated using CytoBank showing decreased CD3 (left), CD4 (middle), and CD8 (right) expression in moderate and severe COVID-19 patients as compared to healthy donors in the CD45⁺ compartment of PBMCs. Here, 5 HDs, 5 moderate COVID-19 patients and 6 severe COVID-19 patient samples from the first day of study enrollment were used to generate the plots. (FIG. 9B). Serial blood draws from our patient cohort enables us to track the CD16^{Int} LDIB population percentage in Ficoll isolated PBMCs over the course of patient hospitalization and correlate it with patient severity and in some cases, clinical outcomes. The first time point indicates enrollment into our study. For the severe patient cohort, samples were collected and analyzed everyday whereas in the moderate cohort, on average, samples were obtained every third day. A red dot indicates that a patient is classified as severe whereas a blue dot signifies a patient is considered moderate. The green line represents the average level of CD16^{Int} neutrophils in healthy patients for a reference of a “normal” level.

FIGS. 10A-10B. Surface marker expression profiling of neutrophils in moderate and severe COVID-19 patients. (FIG. 10A) Representative cluster maps of neutrophil subsets in moderate and severe COVID-19 patients as compared to healthy donors. Here, data from 5 HDs, 5 moderate COVID-19 patients and 6 severe COVID-19 patient samples from the first day of study enrollment were used to generate the plots. (FIG. 10B) Heatmap showing differential surface marker expression of the overall CD66b neutrophil populations in moderate and severe COVID-19 patients as compared to healthy donors.

FIGS. 11A-11B. Differential expression of neutrophil clusters in patients over their clinical course of disease. (FIG. 11A) viSNE plots representing the total CD66b+ neutrophil pool in 4 patients who experienced different clinical courses from days 1, 3 and 5 of study enrollment. Data represents a patient who was classified as severe on days 1, 3 and 5 (top), a patient whose condition improved, and was transitioned to a moderate patient by day 5 (2nd from top), a patient who remained in the moderate group for the entirely of the study (2nd from bottom), and one patient who progressed from the moderate to severe group (bottom). The dynamic nature of CD66b+ neutrophil populations over the course of disease are highlighted by the black and red circles, where cluster surface marker phenotypes are indicated in FIG. 11b. (FIG. 11B) Heatmap showing differential surface marker expression on the CD66b⁺ neutrophil pool, which indicates specific subsets of neutrophil populations within the neutrophil compartment.

FIGS. 124-12B. Trending LDIB population with clinical D-dimer levels. Sequential whole blood analysis of the CD16Int LDIB population (middle circle) for severe (FIG. 12A) and moderate (FIG. 12B) COVID-19 patients overlaid with clinical D-dimer wants from the corresponding days.

FIGS. 13A-13D. The identification of a CD16 intermediate low-density neutrophil population in COVID-19 patients. (FIG. 13A) The averaged percent of CD16 negative (CD16^{Neg)}, CD16 intermediate (CD16^{Int}), and CD16 high (CD16^{high}) neutrophils from serially drawn whole blood samples among healthy donors (HD, n=6), comorbid control patients (Cm Ctrl, n=9), moderate (n=24), and severe n=12) COVID-19 patients. Cells were gated on the CD45⁺CD66b⁺ population. Summarized data and representative dot plots are shown. (FIG. 13B) Cluster maps for moderate and severe COVID-19 patients as compared to HD and Cm Ctrl. The data was generated from CyTOF based analysis of CD45⁺ PBMCs isolated from peripheral blood. (FIG. 13C) Representative dot plots (left) and summarized data (right) showing the overall percent of CD66b⁺ neutrophils (gated on viable, CD45⁺) and the CD16^{Int} subset as found in Ficoll isolated PBMCs analyzed using CyTOF mass cytometry in HD, Cm Ctrl, moderate and severe COVID-19 patients. FIG. 13 (D) Representative viSNE cluster plots show the CD66b (left) and CD16 (right) expression within the CD45⁺ PBMC populations in Cm Ctrl, and patients with moderate and severe COVID-19. Red circles indicate the location of the neutrophil population while blue circles indicate the CD16^{Int} neutrophil population. Data are presented as Mean±SD. p values were determined using a one-way ANOVA with multiple comparisons. **p<0.01, ***p<0.001, ****p<0,0001.

FIGS. 14A-14E. Phenotypic characteristics of neutrophil populations. (FIG. 14A) Wright Giemsa staining of sorted CD66b⁺CD16^{Neg} (left), CD16^{Int} (middle), and CD16^{High} right) populations show different stages of neutrophil maturation. (FIG. 14B) Representative cluster maps of CD66b⁺ neutrophil subsets in moderate and severe COVID-19 patients as compared to Cm Ctrl patients and HDs. (FIG. 14C) Representative histograms showing indicated surface molecule expression levels on CD16^{High} (blue) and CD16^{Int} (red) LDN. Cells were gated on the viable CD66b⁺ population. (FIG. 14D) viSNE cluster plots highlight the expression of CD11b and CD44 in the CD16^{Int} (blue circle) and CD16^{High} (red circle) neutrophil populations, (FIG. 14E) Using mass cytometry, CD44 expression on CD66b⁺ neutrophils from HDs (n=6), Cm Ctrl (n=9), moderate (n=24) and severe (n=12) COVID-19 patients is shown. Representative dot plots and summarized data are shown. Data are presented as Mean±SD. p values were determined using a one-way ANOVA with multiple comparisons. **p<0.01, ***p<0.001.

FIGS. 15A-15E. CD16^{Int} LDN exhibit proinflammatory gene signatures with functionally active phenotype. (FIG. 15A) Volcano plot shows differentially expressed genes (DEGs) between CD16^{Int} and CD16^{High} LDN. (FIG. 15B) Top 20 enriched GO:BP categories for CD16^{Int} versus CD16^{High} LDN from severe COVID-19 patients. (FIG. 15C) The heatmap shows DEGs related to neutrophil degranulation and NET formation, neutrophil phagocytosis, neutrophil signaling, and neutrophil trafficking and function between CD16^{High} and CD16^{Int} LDN. (FIG. 15D) The phagocytic capacity of CD16^{High} and CD16^{Int} neutrophils from whole blood in severe COVID-19 patients (n=4) was assessed using a pHrodo™ Green S. aureus BioParticles™ phagocytosis assay. Gating strategy, representative histogram, and summarized mean fluorescent intensity OHO data are shown. **p<0.01 (student's t-test). (FIG. 15E) Representative confocal image of spontaneous NET formation from sorted CD16^{Int} LDN. Anti-human lactoferrin (shown in red) and neutrophil DNA stained with DAPI (shown in blue), merge image shows NETS characteristic structures. Data are presented as Mean±SD. p values were determined using a Student's t-test. **p<0.01.

FIGS. 16A-16F. CD16^{Int} LDN interact with platelets for activation leading to a hypercoagulable state. (FIG. 16A) GSEA analysis shows significantly enriched pathways including platelet morphogenesis, platelet aggregation, platelet degranulation, and platelet activation in CD16^{Int} LDN compared to CD16^{High} LDN from severe COVID-19 patients (n=3). (FIG. 16B) Representative dot plots showing neutrophil-platelet aggregates. Cells were gated on the CD66b⁺ population. (FIG. 16C) Gated on neutrophil-platelet aggregates, platelet activation marker CD62P expression levels were measured. Representative dot plots and summarized data are shown. (FIG. 16D) Gated on neutrophil-platelet aggregates, CD40 expression levels were detected. Representative dot plots and summarized data are shown. (FIG. 16E) CD40 expression levels on CD16^{Int} LDN from moderate and severe COVID-19 patients. Representative dot plots and summarized data pooled from serial patient draws throughout the course of their hospital admission and grouped according to patient status are shown. A linear mixed effect model was used to determine significance. *p<0.05. (FIG. 16F) D-dimer levels were correlated with the percent of CD40⁺CD16^{Int} neutrophils from longitudinal, serial blood draws measured on the day of sample acquisition. Pearson correlations were used to indicate statistical significance. Data are presented as Mean±SD. p values were determined using a Student's t-test. *p<0.05, **p<0.01.

FIGS. 17A-17E. Comparison of PBMC and BAL fluid immune populations using CyTOF. (FIG. 17A) CD66b⁺ neutrophil populations (left) and CD16 negative, intermediate and high populations (right) were compared in PBMCs and BAL fluid isolated on the same day. (FIG. 17B) The expression of CD44 on CD16^{Int} neutrophils was measured in PBMCs and BAL fluid taken on the same day in 3 severe patients. Representative plots (left) and summarized data (right) are shown. (FIG. 17C) The expression of CXCR3, CD38 (top) and 1E-7RA, LAMP-1 (bottom) was measured on CD16^{Int} neutrophils in PBMCs and BAL fluid. Representative plots and summarized data are shown. (FIG. 17D) Concentration of 20 cytokines/chemokines in the BAL fluid of severe COVID-19 patients (n=6) as measured by U-PLEX assay. (FIG. 17E) Concentration of IP-10, G-CSF, IL-8, and VEGF-A in plasma samples versus BAL fluid of severe COVID-19 patients (n=6) as measured by U-PLEX assay. Data are shown as mean±SD. p values were determined using a Student's t-test *p<0.05, **p<0.01, ***p<0.001, ****p<0.0001.

FIGS. 18A-18E. Enhanced cytokine production by CD16^{Int} LDN in severe COVID-19 patients. (FIG. 18A) Plasma concentrations of IL-6 and INF-α in a single draw from HDs (n=6) and Cm Ctrl (n=9), and the average value during study enrollment: for moderate (n=24), and severe (n=12) COVID-19 patients. (FIG. 18B, 18C) IL-6 and TNF-α levels in serial patient draws were then correlated with both the percent of total neutrophils (FIG. 18B) and the percent of CD16^{Int} neutrophils (FIG. 18C) in the corresponding sample as measured by CyTOF. (FIG. 180) Representative dot plots of TNF-α (top) and IL-6 (bottom) production from LPS stimulated neutrophils cultured from whole blood samples of Cm Ctrl (n=8), moderate (n=3) and severe patients (n=2), with accompanying summarized data. (FIG. 18E) Pie charts show the relative contribution of neutrophils to the total TNF-α and IL-6 ex vivo pool as compared to all other immune cells in Cm Ctrl patients and severe COVID-19 patients. Pearson correlations were used to indicate statistical significance in all correlations. Data are shown as mean-±SM. p values were determined using a one-way ANOVA with multiple comparisons. *p<0.05. ****p<0.0001

FIGS. 19A-19F. Correlations of clinical coagulation and systemic inflammation indicators and disease outcomes with CD16^{Int} LDN. (FIG. 19A) For severe and moderate patients, the clinical values of D-dimer, ferritin, platelets and LDH were acquired from patient charts. The average value of serial blood draws from patients were used. An unpaired student's t-test was used to determine significance. *p<0.05 (FIGS. 19B, 19C) The D-dimer, ferritin, platelet number and LDH levels for all COVID-19 patient samples were correlated with the total CD66b⁺ neutrophil percentage (FIG. 19B) or CD16^{Int} (FIG. 19C) neutrophils in the PBMCs. For all correlation data, a line of best fit is shown to visually examine correlation, with a green line representing a statistically significant correlation and a red line representing a non-significant correlation. Pearson correlations were used to determine statistical significance in all correlations, where *p<0.05. (FIGS. 19D-F) Longitudinal, serial blood draws from our patient cohort (25 patients) enables us to track the CD16^{Int} LDN population percentage in Nicoll isolated PBMCs over the course of patient hospitalization and correlate it with patient clinical outcomes. (FIG. 19D) COVID-19 patients (n=10) with mortality show an increased CD16^{Int} LDN trend over time. (FIG. 19E) COVID-19 patients (n=6) with convalescence show a decreased CD16^{Int} LDN trend over time and the frequency of CD16^{Int} LDN in the blood draw before discharge is similar to the level in heathy donors (HD, n=6) or comorbidity control patients (Cm Ctrl, n=9). (FIG. 19F) COVID-19 patients (n=9) with convalescence show low levels of CD16^{Int} LDN similar to those from HD over time. Dotted line represents the average level of CD16^{Int} LDN in PBMC from Fir) (n=6). All data are shown as mean±SD.

FIGS. 20A-20B. COVID-19 patients have increased neutrophils and neutrophil to lymphocyte ratio (NLR). (FIG. 20A) Neutrophil and lymphocyte percentages and the NLR in whole blood as measured by a clinical complete blood count (CBC) in healthy donors (HD), comorbidity control patients (Cm Ctrl), and patients with moderate and severe COVID-19. Data points represent a single time point collected from 6 HDs, 9 Cm Ctrl, and the average values of serial blood samples collected during patient hospitalization from 24 moderate patients and 12 severe patients starting from the day of enrollment. Pie charts depict representative data of the NLR in HDs, severe and moderate patients. (FIG. 20B) Representative viSNE plots generated using CytoBank showing decreased CD3 (left), CD4 (middle), and CD8 (right) expression in Cm Ctrl patients, moderate and severe COVID-19 patients as compared to HDs in the CD45⁺ compartment of PBMCs. Data are presented as mean±SD. p values were determined using a one-way ANOVA with multiple comparisons. *p<0.05, **p<0.01, ***p<0.001.

FIG. 21. Cluster analysis of neutrophils within the CD45+ PBMCs in HD, Cm Ctrl, and moderate and severe COVID-19 patients. Heatmap of differential expression pattern of lineage and surface markers of neutrophils in PBMCs. The color key identifies the cluster populations.

FIGS. 22A-22B. Differential expression of neutrophil clusters in patients over their clinical course of disease. (FIG. 22A) viSNE plots representing the total CD66b⁺ neutrophil pool in 4 patients who experienced different clinical courses from days 1, 3 and 5 of study enrollment. Data represent a patient who was classified as severe on days 1, 3 and 5 (top), a patient whose condition improved, and was transitioned to a moderate patient by day 5 (2^{nd} from top), a patient who remained in the moderate group for the entirely of the study (2^{nd} from bottom), and one patient who progressed from the moderate to severe group (bottom). The dynamic nature of CD66b⁺ neutrophil populations over the course of disease are highlighted by the black and red circles, where cluster surface marker phenotypes are indicated in S4b. FIG. 22B) Heatmap showing differential surface marker expression on the CD66b⁺ neutrophil pool, which indicates specific subsets of neutrophil populations within the neutrophil compartment.

FIGS. 23A-23C. Differentially expressed genes and enriched pathways between CD16^{High} and CD16^{Int} LON from severe COVID-19 patients, (FIG. 23A) Principal component analysis (PCA) by the first two principal components (PC1: 68%; PC2: 18%). CD16^{High} and CD16^{Int} LDN were sorted from three severe COVID-19 patients. Normal density neutrophils (NDN) were obtained from three HDs. The three sample groups segregate from each other with a high aggregation between replicates. (FIG. 23B) Heatmaps show differentially expressed genes for GO: neutrophil activation (left) and GO: neutrophil involved immune response (right) between CD16^{High} and CD16^{Int} LDN. (FIG. 23C) GSEA analysis shows significant enriched pathways in CD16Int LDN compared to CD16^{High} LDN.

FIGS. 24A-24B. Correlation of plasma levels of cytokine/chemokine with the frequency of CD16^{Int} LDN in the PBMC population. (FIG. 24A) CXCR3 and CD44 expression levels on CD16^{Int} and CD16^{High} neutrophils in BAL fluid samples. Data are shown as mean±SD. p values were determined using a Student's t-test *p<0.05, **p<0.01. (FIG. 24B) Plasma IL-10, IL-1RA, MCP-1 and MIP-1a levels in serial patient draws were correlated with both the percent of CD16^{High} and CD16^{Int} neutrophils in the corresponding sample as measured by CyTOF. Pearson correlations were used to indicate statistical significance in all correlations, where ns=p≥0.05, *p<0.05, **p<0.01., ***p<0.001.

FIGS. 25A-25B. Correlation of TNF-α and IL-6 with clinical markers (FIG. 25A) TNF-α plasma concentrations were correlated with the clinically measured values from the same day that the sample was acquired. Samples that fell below the level of detection of the TNF-α ELISA were excluded from correlation data. (FIG. 25B) IL-6 plasma concentrations were correlated with the clinically measured D-dimer, ferritin, platelets, and LDH levels from the same day that the sample was acquired. Samples that tell below the level of detection of the IL-6 ELISA were excluded from correlation data. For all correlation data, a line of best fit is shown to visually examine correlation, with a green line representing a statistically significant correlation, a red line representing a non-significant correlation. Pearson correlations were used to determine significance. *p<0.05, **p<0.01, ***p<0.001.

FIGS. 26A-26B. Association of circulating CD16^{Int} neutrophil population with clinical D-dimer levels. Sequential whole blood analysis of the CD16^{Int} neutrophil population (middle circle) for severe (FIG. 26A) and moderate (FIG. 26B) COVID-19 patients overlaid with clinical D-dimer quants from the corresponding days.

### DETAILED DESCRIPTION

Severe acute respiratory syndrome coronavirus 2 (SARS-CoV-2) is a novel viral pathogen that causes a clinical disease called coronavirus disease 2019 (COVID-19). Approximately 20% of infected patients experience a severe manifestation of the disease, causing bilateral pneumonia and acute respiratory distress syndrome. Severe COVID-19 patients also have a pronounced coagulopathy with approximately 30% of patients experiencing thromboembolic complications. However, the etiology driving the coagulopathy remains unknown. It was explored whether the prominent netarophilia seen in severe COVID-19 patients contributes to inflammation-associated coagulation. It was found in severe patients the emergence of a CD16^{Int}CD44^{low}CD11b^{Int} low-density inflammatory band (LDIB) neutrophil population that trends over time with changes in disease status. These cells demonstrated spontaneous neutrophil extracellular trap (NET) formation, phagocytic capacity, enhanced. cytokine production, and associated clinically with D-dimer and systemic IL-6 and TNF-α levels, particularly for CD40⁺ LDIBs. It was concluded that the LDIB subset contributes to COVID-19-associated coagulopathy (CAC) and could be used as an adjunct clinical marker to monitor disease status and progression. Identifying patients who are trending towards LDIB crisis and implementing early, appropriate treatment could improve all-cause mortality rates for severe COVID-19 patients.

### Methods of Treatment

In certain aspects, methods are provided for treating coronavirus disease 2019 (COVID-19) in a subject, comprising the step of administering to the subject a therapeutically effective therapeutic agent, wherein the therapeutic agent inhibits CD66b⁺CD16^{Int}CD11b^{Int}CD44^{low}CD40⁺ low-density inflammatory band (LD1B) neutrophil population.

In certain aspects, methods are provided for treating coronavirus disease 2019 (COVID-19) in a subject, comprising the step of administering to the subject a therapeutically effective therapeutic agent, wherein the therapeutic agent inhibits COVID-19-associated coagulopathy (CAC).

In certain aspects, methods are provided for treating coronavirus disease 2019 (COVID-19) in a subject, comprising the step of administering to the subject a therapeutically effective therapeutic agent, wherein the subject has a lower level of CD16^{Int}CD44^{Low}CD11b^{Int} low-density neutrophils, and wherein the therapeutic agent is respiratory therapy.

In certain aspects, methods are provided for treating a subject having been diagnosed with coronavirus disease 2019 (COVID-19) with a therapeutic agent that inhibits low-density inflammatory neutrophil (LDN) population expressing intermediate levels of CD16 (CD16^{Int}).

In certain aspects, the subject has an elevated plasma level of IL-6 as compared to a control.

In certain aspects, the LDN are CD66b⁺ LDN.

In certain aspects, the subject has elevated plasma levels of IL-10, RA, MCP-1 and/or MIP-1α as compared to a control.

In certain aspects, the subject has an elevated plasma level of IL-6 and/or TNF-α as compared to a control.

In certain aspects, the subject has an elevated plasma level of D-dimer as compared to a control.

In certain aspects, the subject has an elevated plasma level of ferritin as compared to a control.

In certain aspects, the subject has an elevated plasma level of D-dimer and ferritin.

In certain aspects, the subject is treated with a cytokine blocking antibody. In certain embodiments, the cytokine blocking antibody is tocilizumab, adalimurnab, or etanercept.

In certain aspects, the subject is treated with an immunosuppressive regimen.

In certain aspects, the subject is treated with dexamethasone or anti-IL-6 therapy.

In certain aspects, the subject is treated with antibiotics, fluids, zinc, vitamins, antiviral medications, vasopressors, inotropes, inhalational agents, antihypertensives, diabetic medications, ulcer prophylaxis, and other prescribed agents.

in certain aspects, the therapeutic agent inhibits LDN by at least 50%. Typically, the therapeutic agent is administered in a concentration range of about 1 mg/kg of the subject's body weight to about 10 mg/kg per day.

As is well known in the art, the methods of the present invention may be administered orally or intravenously.

As used herein, “treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and improved prognosis.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

As used herein, the term “about”, unless the context dictates otherwise, is used to mean a range of +or −10%.

### Methods of Detection

In certain embodiments, the present invention provides a method of detecting the severity level of coronavirus disease 2019 (COVID-19) in a subject, comprising measuring the level of CD16^{Int} low-density inflammatory neutrophil (LDN) in plasma as compared to a control.

The invention will now be illustrated by the following non-limiting Examples.

### EXAMPLE 1

### Emergence of Low-Density Inflammatory Neutrophils Correlates with Hypercoagulable State and Disease Severity in COVID-19 Patients

Severe acute respiratory syndrome coronavirus 2 (SARS-CoV-2) is a novel viral pathogen that causes a clinical disease called coronavirus disease 2019 (COVID-19). Approximately 20% of infected patients experience a severe manifestation of the disease, causing bilateral pneumonia and acute respiratory distress syndrome. Severe COVID-19 patients also have a pronounced coagulopathy with approximately 30% of patients experiencing thromboembolic complications. However, the etiology driving the coagulopathy remains unknown. Here, we explore whether the prominent neutrophilia seen in severe COVID-19 patients contributes to inflammation-associated coagulation. We found in severe patients the emergence of a CD16^{Int}CD44^{low}CD11b^{Int} low-density inflammatory band (LDIB) neutrophil population that trends over time with changes in disease status. These cells demonstrated spontaneous neutrophil extracellular trap (NET) formation, phagocytic capacity, enhanced. cytokine production, and associated clinically with D-dimer and systemic IL-6 and TN17-α levels, particularly for CD40⁺ LDIBs. We conclude that the LDIB subset contributes to COVID-19-associated coagulopathy (CAC) and could be used as an adjunct clinical marker to monitor disease status and progression. Identifying patients who are trending towards LDIB crisis and implementing early, appropriate treatment could improve all-cause mortality rates for severe COVID-19 patients.

In addition to significant pulmonary complications, severe COVID-19 patients also have a notable coagulopathy. Multiple studies report COVID-19 patients experiencing thromboembolic events including myocardial infarction, pulmonary embolism, cerebrovascular accident, and deep vein thromboses. The majority of patients with severe disease have increased. D-dimers, platelet abnormalities, and decreased prothrombin time (PT) or partial thromboplastin time (PTT) over the course of their hospitalization. Given the prevalence of thromboembolic complications in severe COVID-19 patients, the standard of cafe for intubated patients now includes full anticoagulation therapy. However, the etiology of the coagulopathy has yet to be clearly elucidated. In this study, we investigate the hypothesis that the excessive neutrophilia seen in severe COVID-19 patients directly contributes to COVID-19-associated coagulopathy (CAC). We found that the most severe patients, requiring mechanical ventilation, demonstrated. a marked increase in the overall CD66b⁺ neutrophil percentage within the peripheral blood compartment as compared to moderate patients. Within the severe COVID-19 patient cohort, we also saw the emergence of a significant population of CD16^{Int}CD44^{Low}CD11b^{Int} low-density neutrophils, which we refer to as low-density inflammatory band cells (LDIBs). The increases in this population trended with disease severity and mortality while decreases were associated with extubation and discharge. Additionally, the LDIB population percentage trended with D-dimer levels across all COVID-19 patients. Functional analysis of these cells revealed their phagocytic activity, spontaneous formation of neutrophil extracellular traps (NETs), and enhanced secretion of IL-6 and TNT-α. Plasma levels of IL-6 in all COVID-19 patients positively correlated with the LDIB population while TNF-α showed a trending correlation. Taken together, these findings suggest that LDIBs significantly contribute to CAC.

### RESULTS

### Neutrophil profiling in hospitalized COVID-19 Patients

For our study, we enrolled a total of 13 patients that had tested positive for SARS-CoV-2 by nasopharyngeal swab. Seven patients were initially enrolled in the severe category as defined by necessity of mechanical ventilation within the intensive care unit (ICU) and six were initially enrolled in the moderate group, as patients that had been admitted to the hospital but were not on a mechanical ventilator. The patient demographics was summarized in Table 1. The average age of COVID-19 patients was 66.8 with a male to female ratio of 8:5. Of note, 5/7 severe patients (71.4%) and 3/6 moderate patients (50%) experienced a thromboembolic complication either as a presenting illness or during the course of their hospitalization. Peripheral blood samples were drawn daily from either a venous or arterial line for severe patients whereas moderate patients had samples drawn from a venous line approximately every two to three days.

TABLE 1
Study participant demographics

| | Total | Healthy | COVID-19 |
|---|---|---|---|
| Participants | 19 | 6 | 13 |
| Age, mean, years | 61.26 (28-95) | 50 (28-68) | 66.8 (28-95) |
| Male:Female | 12:7 | 4:2 | 8:5 |
| Race | | 5 Caucasian | 8 Caucasian |
| | | 1 Asian | 5 Black/African |
| | | | American |
| Mean Comorbidities, | | .3 ± .47 | 4 ± 2.0 |
| St Deviation | | | |
| Patients experiencing | | | 8 (61.5%) |
| thromboembolic | | | |
| complications during | | | |
| hospital stay | | | |
| Patients receiving | | | 6 (46%) |
| Hydroxychloroquine + | | | |
| Azythromycin | | | |
| Patients receiving | | | 4 (30.7%) |
| convalescent plasma | | | |
| Mortality | | | 4 (30.7%) |

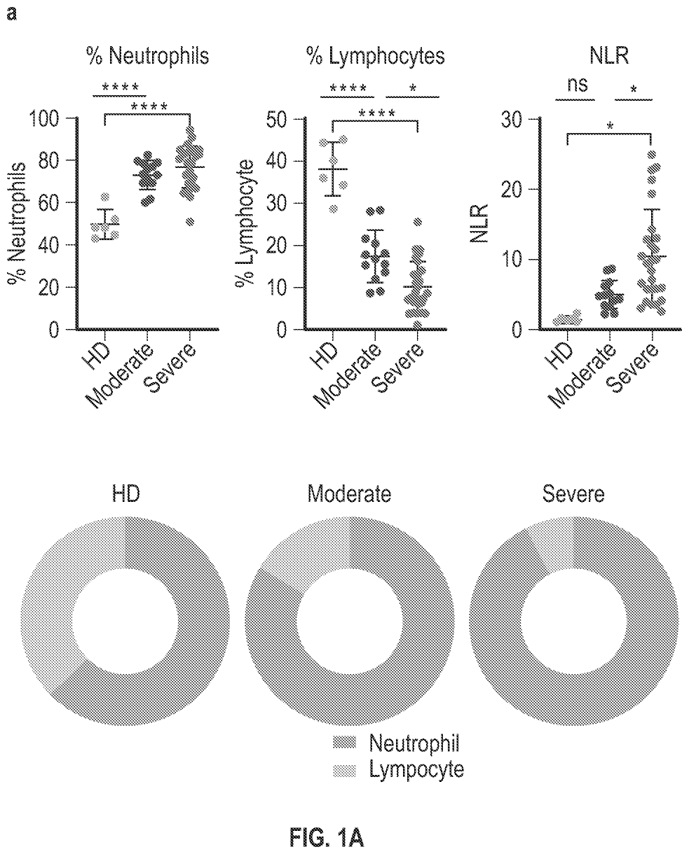

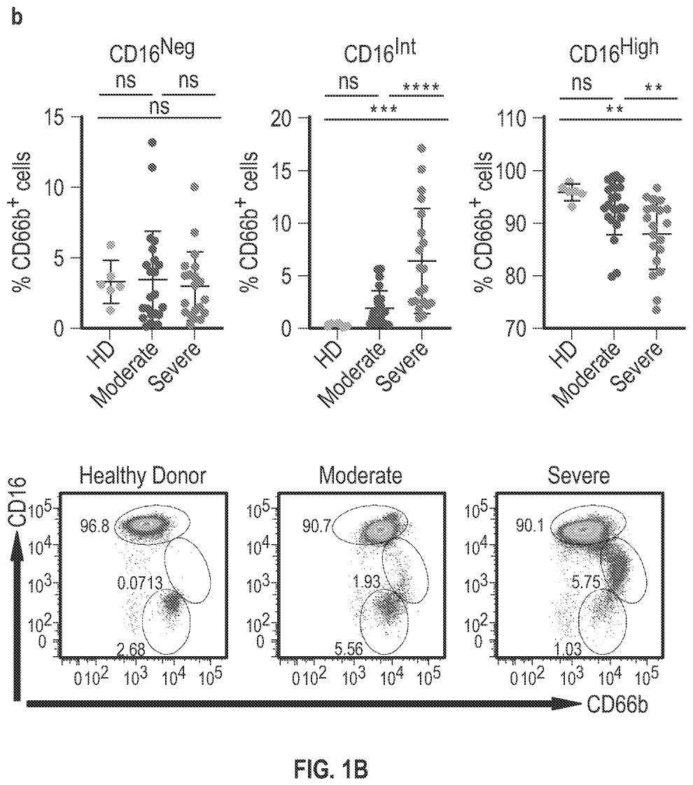

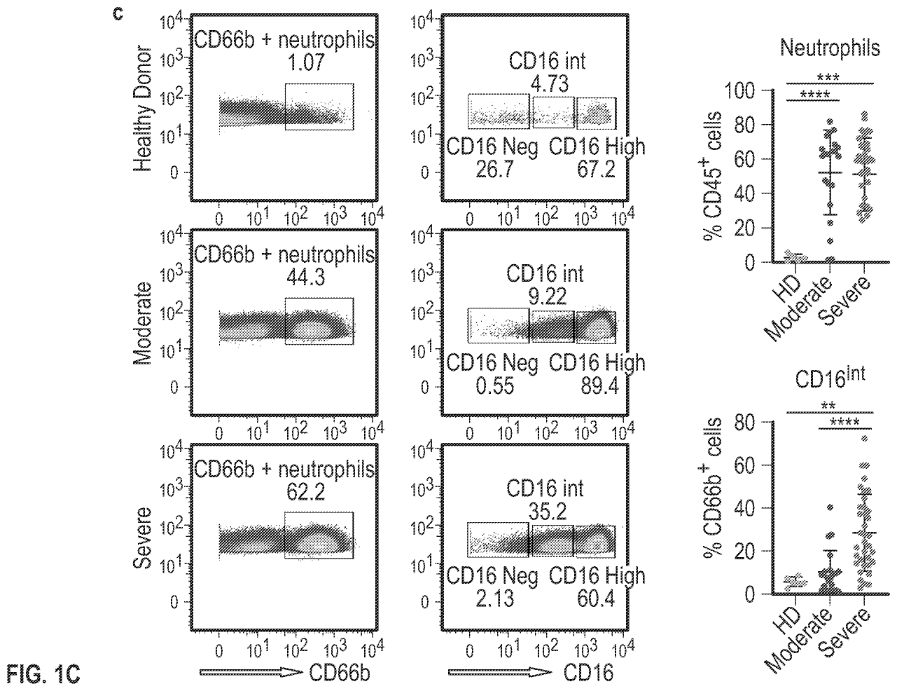

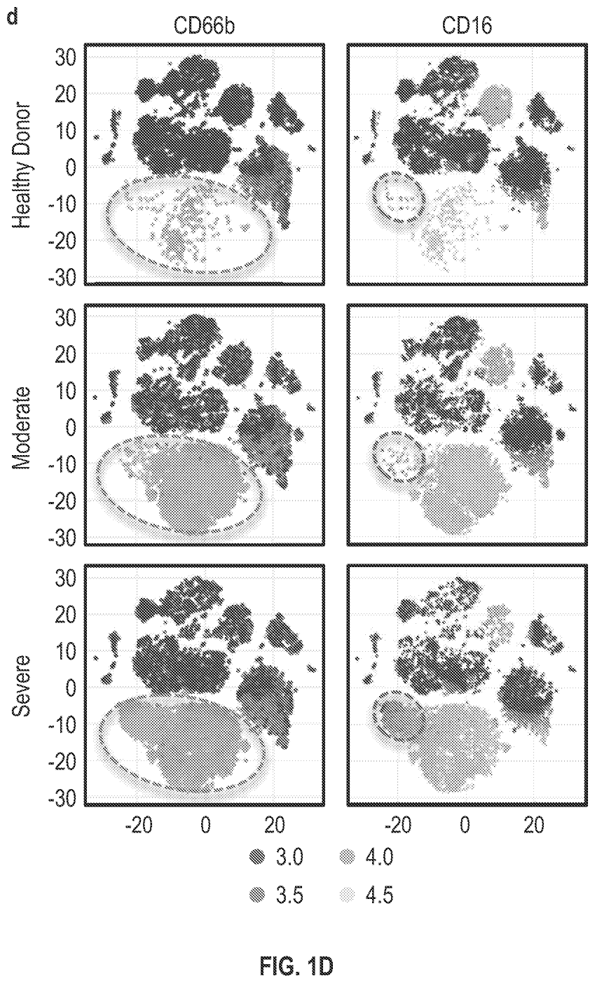

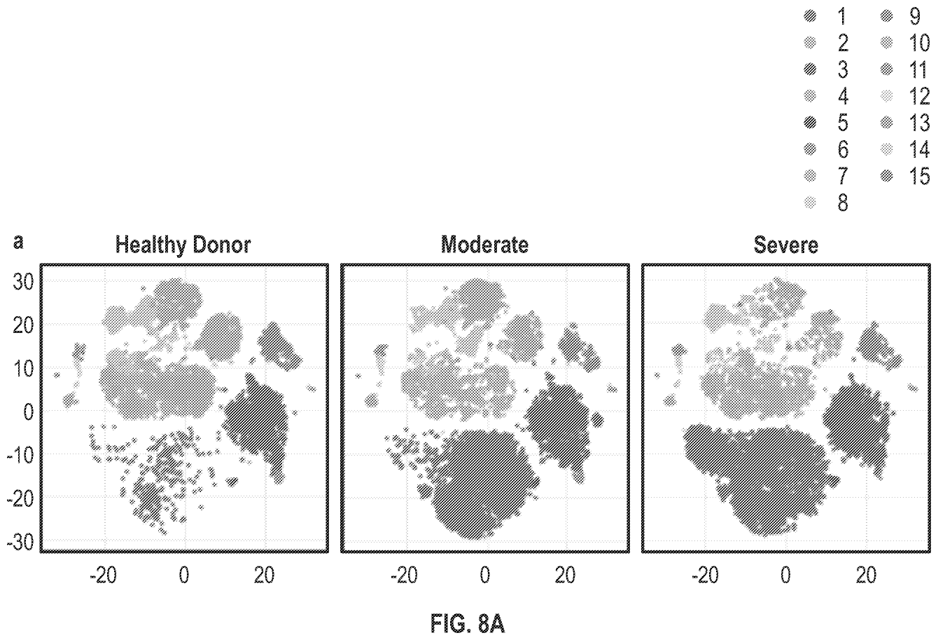

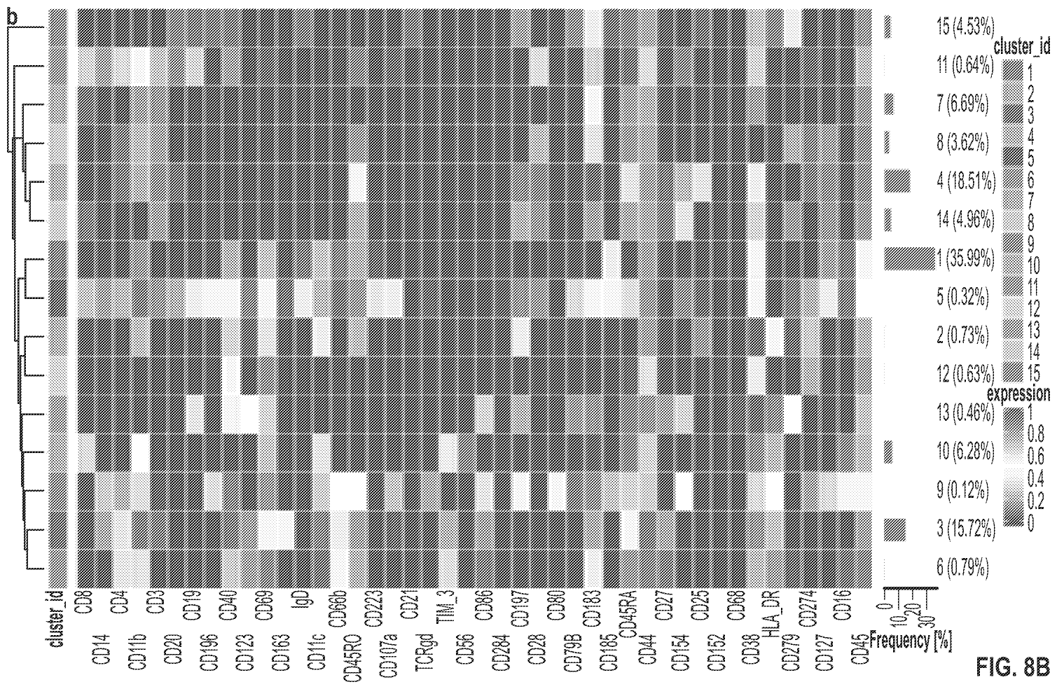

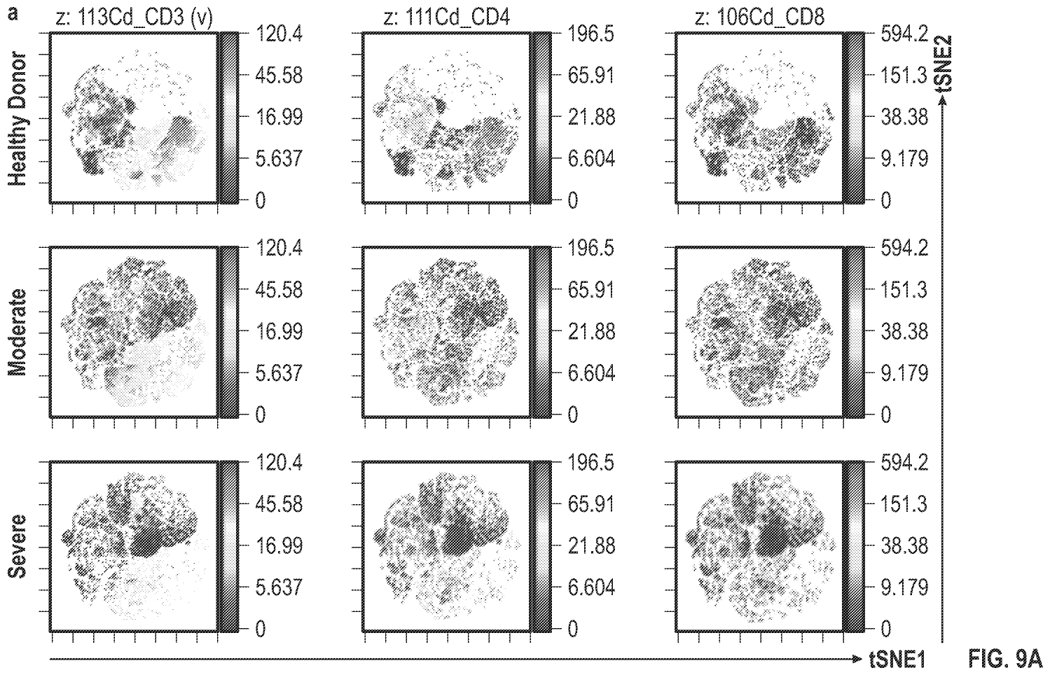

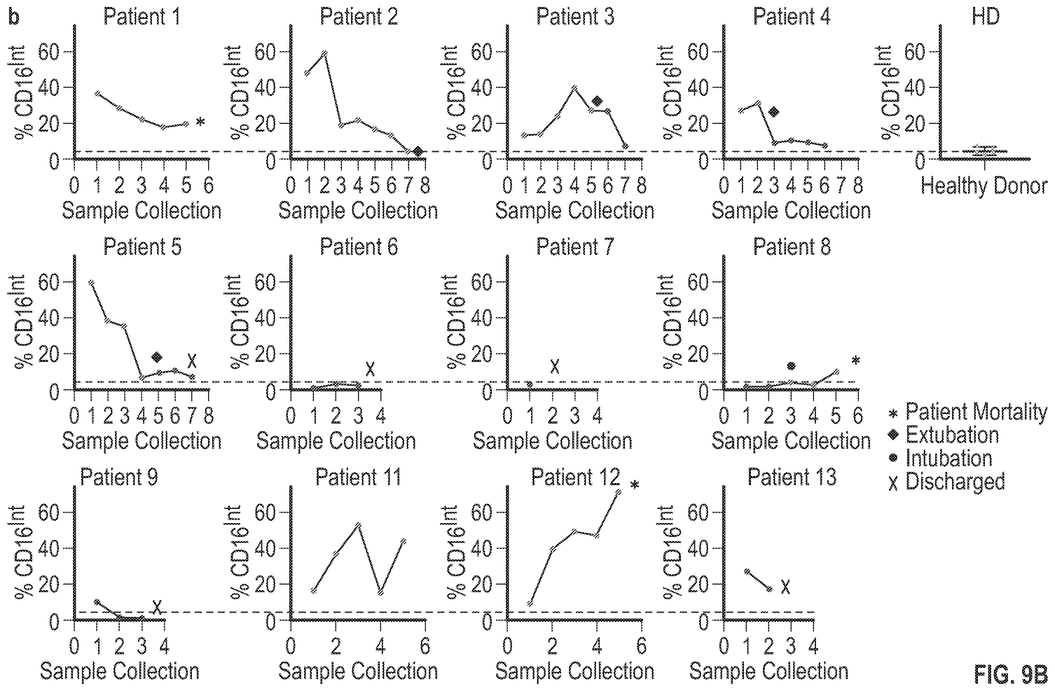

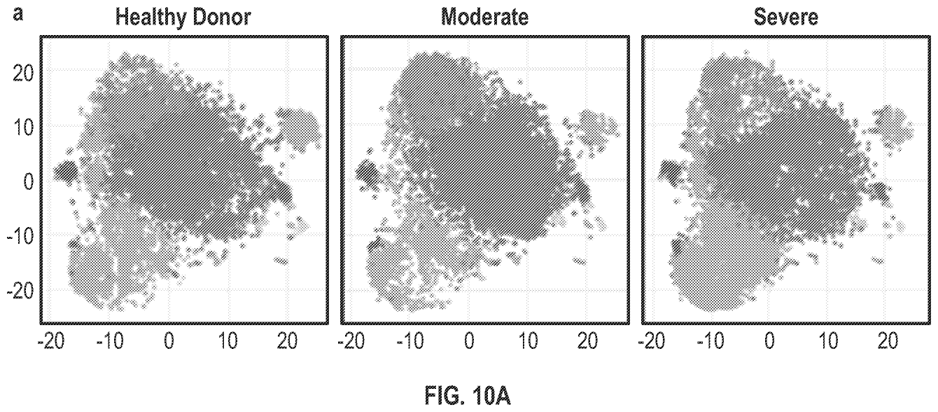

We began our study by comparing the CD45⁺ lineage clusters between healthy donors, moderate, and severe COVID-19 patients. Cell lineage cluster analysis demonstrated that CD66b⁺CD16⁺ neutrophils (cluster 1, FIG. 8B) were the most prominent population in COVID-19 patients which agrees with previous reports indicating a dominant neutrophilia in these patients. We confirmed our results with data pooled from patient serial whole blood. complete blood count (CBC) reports. This data demonstrated that severe patients had approximately a 10% increase in neutrophil percentage in their peripheral blood as compared to moderate patients, and a 30% increase over healthy donors (FIG. 1a). Conversely, the overall lymphocyte percentage in these patients was decreased as compared to the moderate cohort and healthy donors. viSNE analysis of the overall CD3⁺ T cells and CD4⁺ and CD8⁺ T cell subsets showed decreasing population size in patients with moderate and severe COVID-19 as compared to healthy donors (FIG. 9a). Taken together, this data ultimately characterizes an increased NLR within our severe cohort (FIG. 1a) that agrees with previously published reports.

Further investigation into the neutrophil pool revealed three distinct subpopulations within whole blood samples that clustered by CD16^{Neg}, CD16^{Int}, and CD16^{High} expression. Severe COVID-19 patients showed a marked increase in the CD16^{Int} subset, which was significantly lower in the moderate cohort, and virtually absent in the healthy donors (FIG. 1b). CD16^{Int} neutrophils classically have been reported to be low-density neutrophils or immature neutrophils. Clinically, immature neutrophils are called band cells and are associated with a left shift on a complete blood count (CBC). These neutrophils are often mononucleated and smaller than typical neutrophils. Therefore, due to the combination of their number and smaller mononucleated morphology, we were able to pull down a significant portion of these cells from the blood using a typical PBMC Ficoll isolation method. Previous reports also described this phenomenon in more severe cases of autoimmunity. Minimal neutrophils were isolated from healthy donors using this method indicating the unique characteristics of these LDIBs in COVID-19 patients. Isolating the LDIBs via Ficoll resulted in about ˜6-fold enrichment of these cells over peripheral blood within each cohort (FIG. 1c). Therefore, while the actual percentage was higher than in whole blood (FIG. 1b), the ratio between the cohorts was similar thus allowing for valid comparisons. Cluster analysis of isolated PBMCs from a single blood draw in each donor indicated a predominate neutrophil population (circled in red) within the CD45⁺ compartment in the severe COVID-19 cohort as compared to moderate patients and healthy donors (FIG. 1d, left panel). Additionally, in the severe patients, there was a subset of the neutrophil population that expressed intermediate CD16 (blue circle) which was diminished in both the moderate and healthy donors (FIG. 1d, right panel). This adjacent CD16^{Int} cluster represented the LDIB population seen in severe COVID-19 patients (FIG. 1c).

Interestingly, tracking the CD16^{Int} LDIB population over the course of each patients' individual hospital stay revealed an important association between clinical outcomes and the percentage of CD16^{Int} neutrophils (FIG. 9b). Specifically, in patients 3.4, and 5, the percentage of CD16^{Int} cells trended with improvements in disease status. As their CD16^{Int} percentage began to decline, these patients were extubated and switched from the severe to moderate group. Conversely, in patients 1, 8, 12, patient mortality was directly associated with an increasing CD16^{Int} percentage as compared to their baseline at enrollment or the CD16^{Int} neutrophil percentage stayed constantly at a high level (patient 1). Lastly, patients 6, 7, and 9 in the moderate group consistently had a minimal CD16^{Int} population for the duration of the hospitalization prior to their discharge. Collectively, these findings suggest that the most severe COVID-19 patients experienced an emergence of LDIB population that trends with both improvements and declines in patient status.

### Phenotypic Characterization of CD16^{Int} LDIB Cells

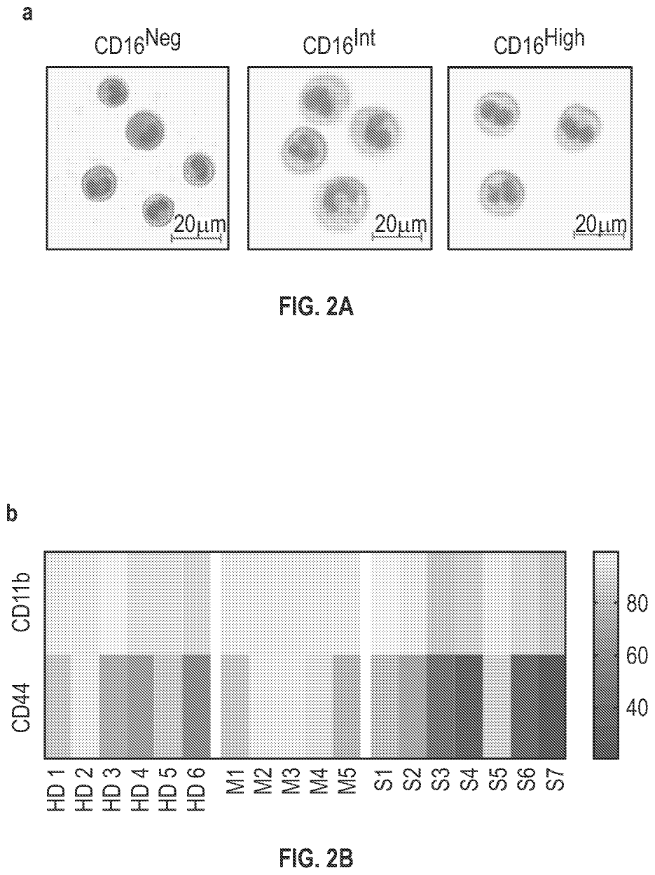

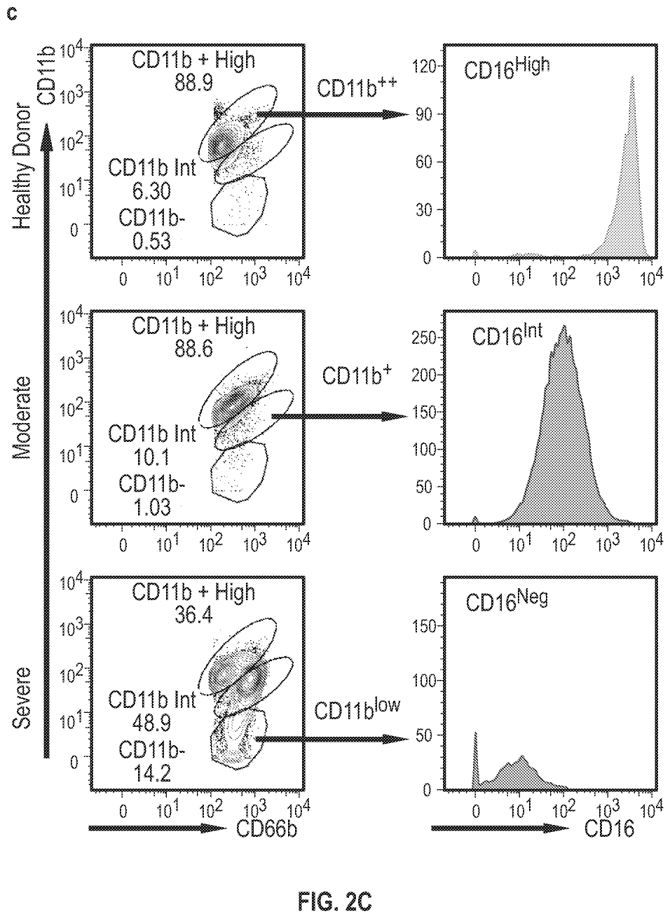

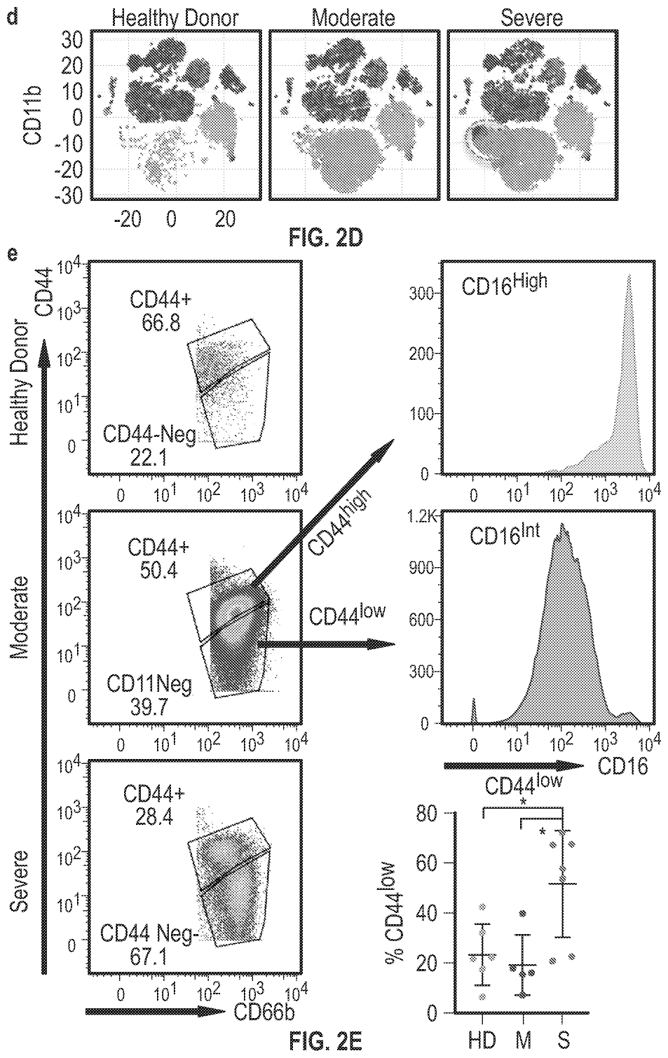

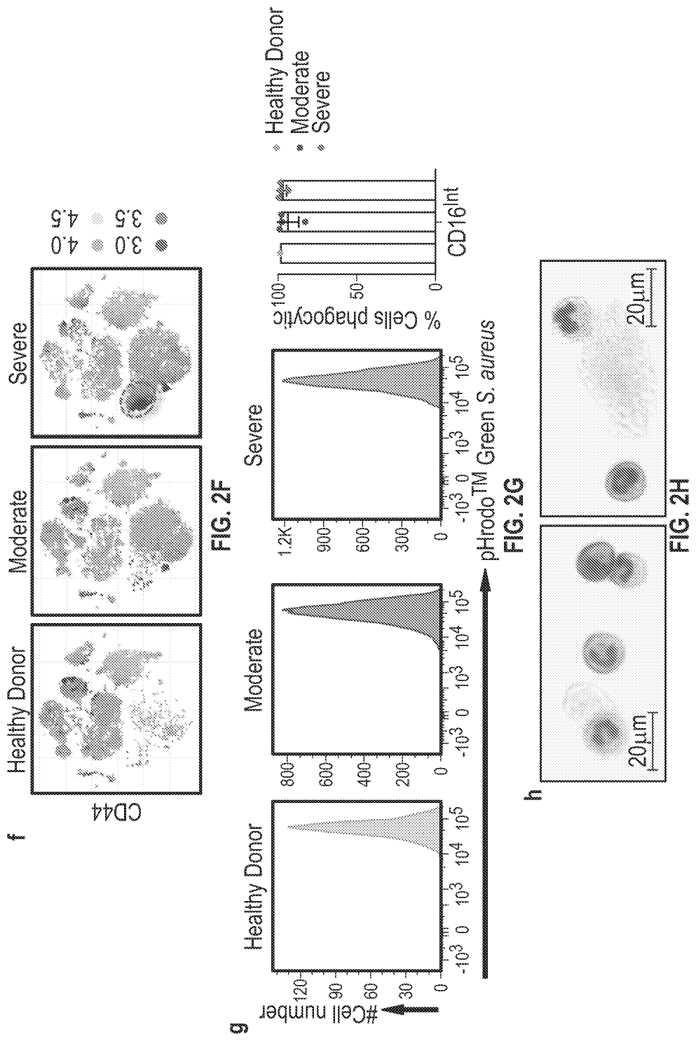

Maturation of neutrophils from hematopoietic stein cells is identified by stages with distinct morphological characteristics. We performed Wright-Giemsa staining to determine if the three CD16 populations of neutrophils were actually neutrophils in the later three stages of development: myelocyte, metamyleocyte (band cell), and granulocyte (mature neutrophil). FIG. 2a clearly showed that the CD16^{Neg} cells were basophilic myelocytes with an ovoid nucleus, the CD16^{Int} cells were band cells with the characteristic band shaped nucleus, and the CD16^{High} cells were segmented, mature neutrophils. However, it is relevant to note that the mature CD16^{High} neutrophils are bi-lobed rather than hyper-segmented and closely resemble pseudo-Pelger-Huet cells. Pseudo-Pelger Huet cells have been described in other severe infections like influenza A, tuberculosis, and human immunodeficiency virus (HIV). It has been suggested that these cells develop as a result of excessive exposure to inflammatory factors like TNT-α and IFN-γ.

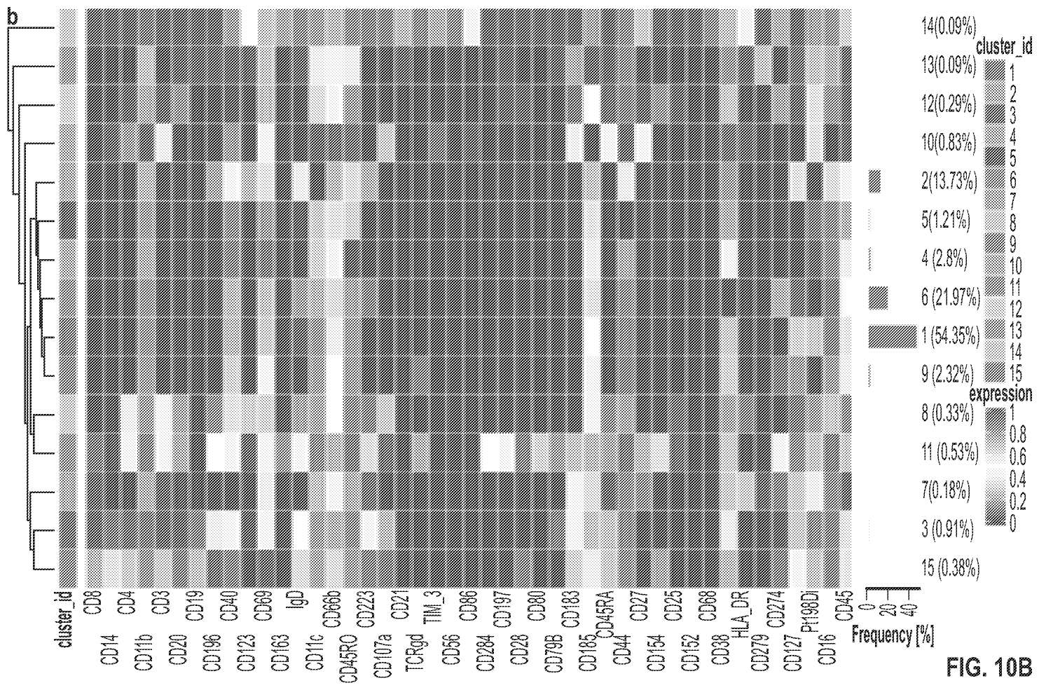

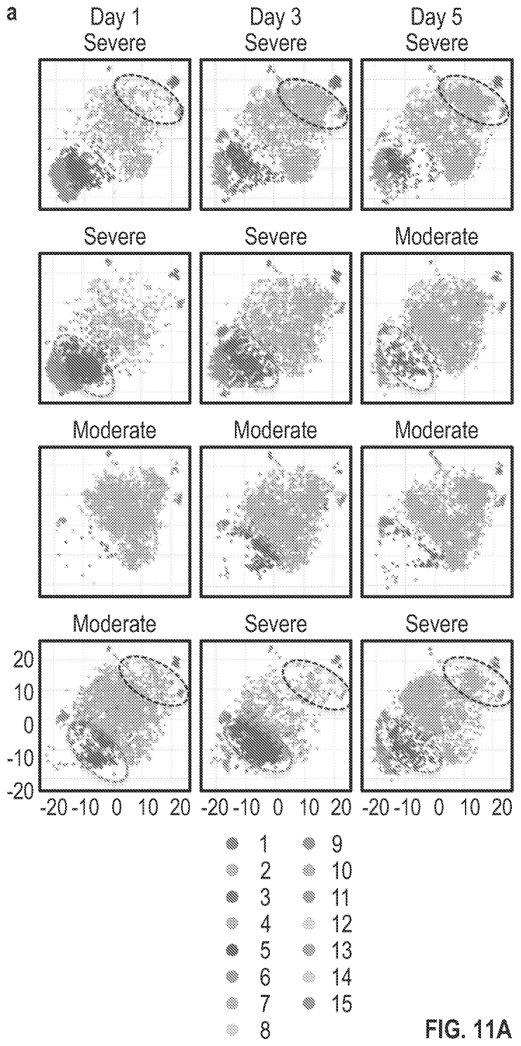

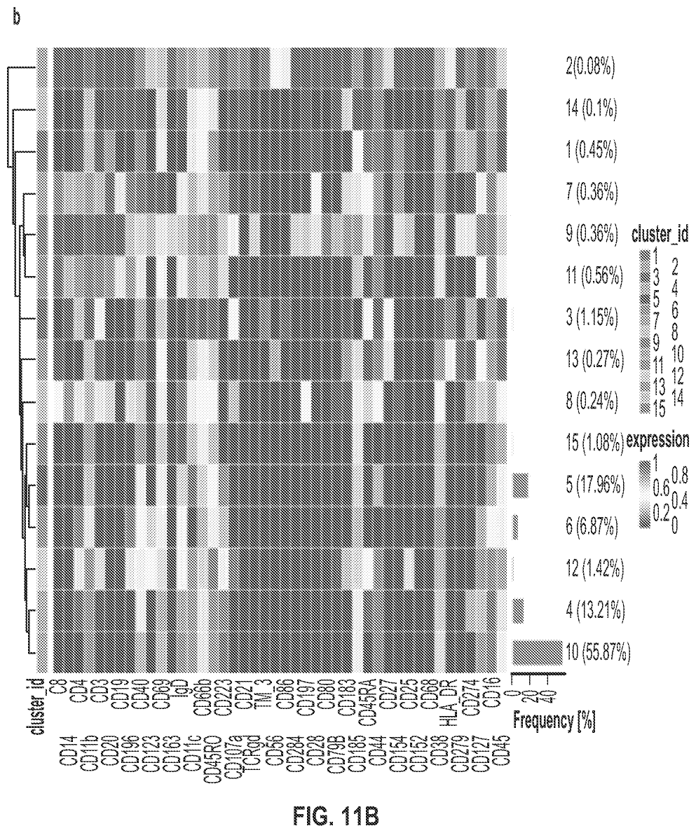

Next, we explored differential surface marker expression on the different CD16 subsets of neutrophils in COVID-19 patients. We first performed cluster analysis on the overall CD66b⁺ neutrophil population. As shown in FIG. 10a, there was an increased prevalence of cluster 2 in the severe patient cohort as compared to moderate and healthy donors. Conversely, there was a slight decrease in density of cluster 1 in the severe group as compared to the other two. Utilizing the heatmap FIG. 10b revealed that cluster 1 expressed high levels of CD11b, CD44 and CD16. Conversely, cluster 2 showed decreased expression of CD44, CD16, and CD11b. Interestingly, tracking the neutrophil clusters in serial blood draws over 5 days from different types of patients revealed the dynamic nature of neutrophil pools in COVID-19 infection (FIGS. 11a, 11b). In the severe patient, over the time, the light blue population (cluster 4, black circle) increased while all the other clusters remained similar. For the moderate patient, the majority of clusters remained stable over time. The patient that was initially enrolled in the severe cohort but changed to moderate by day 5, had a profound decrease in cluster 5 (red circle) over time. Conversely, in the patient that transitioned from moderate to severe, the light blue (cluster 4) and purple clusters (cluster 5) increased over the time, which was consistent with the change in disease severity (FIG. 11a).

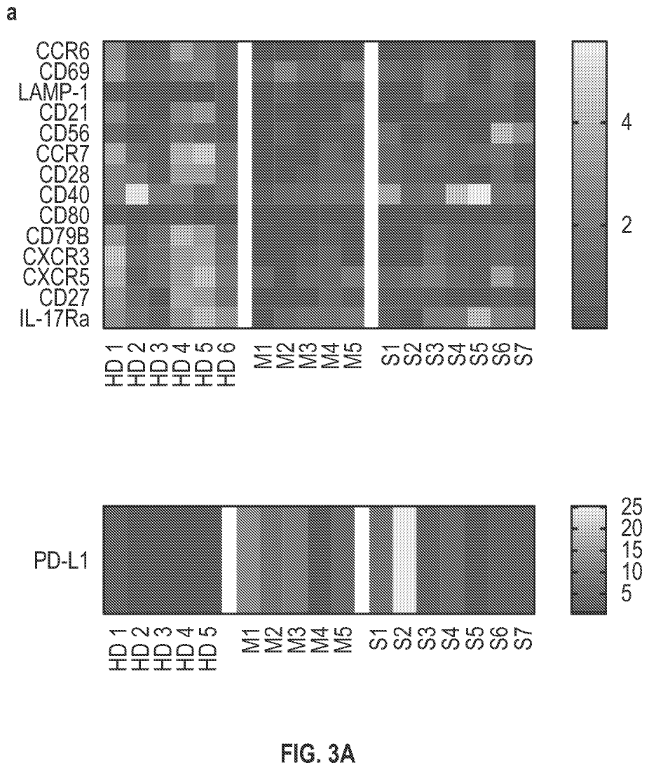

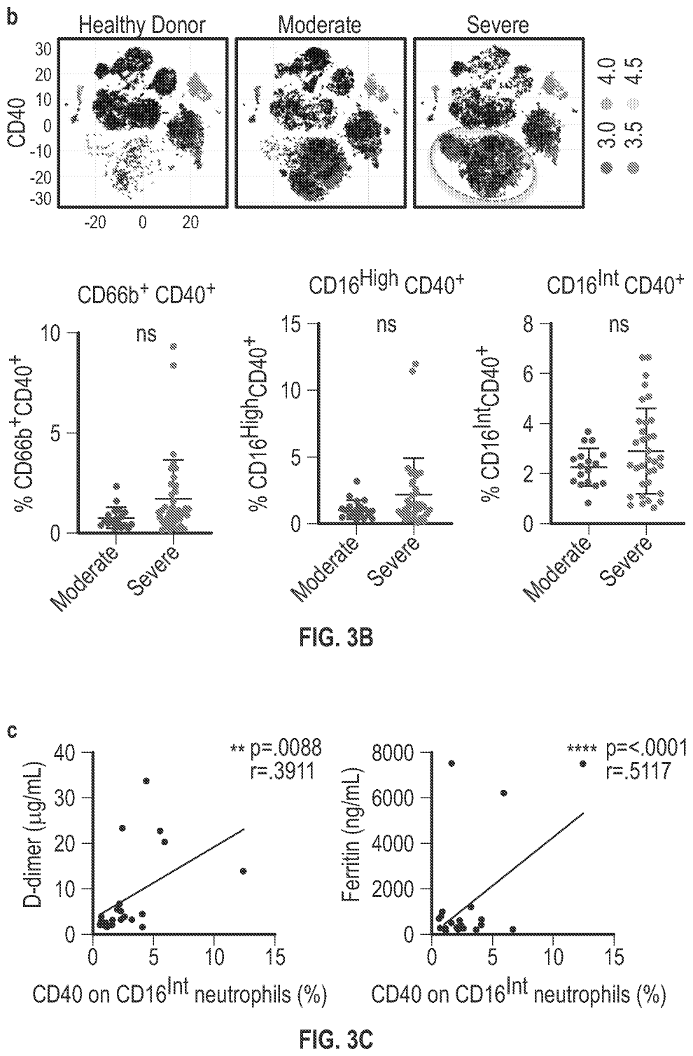

Understanding that the profile of neutrophil clusters associates with disease status, we wanted to expand upon the findings from our analysis and determine a specific surface marker phenotype for three CD16 neutrophil clusters. To do this, we generated a heatmap from the CyTOF analysis profiling the CD66⁺ population within the three cohorts (FIG. 3a). Two markers from our cluster analysis, CD11b and CD44, stood out to be differentially expressed between the healthy donors and the two patient cohorts (FIG. 2b). CD11b expression level was intermediate in the severe cohort while CD44 was the lowest in this patient population. Breaking CD11b expression down by CD16 subset, showed increasing expression of CD11b as the cells progress through the developmental stages, with the LDIBs haying an intermediate expression profile (FIG. 2c). Cluster analysis revealed that the representative LIMB cluster indeed showed decreased CD11b expression (red circle) as compared to the overall CD66b⁺ neutrophil cluster (FIG. 2d).

CD44 is an important surface marker that has been associated with neutrophilic lung inflammation in bacterial pneumonia. Decreased surface expression of CD44 resulted in increased accumulation of neutrophils in the lungs of E. coli infected mice. Therefore, given the known accumulation of neutrophils in the lungs of severe COVID-19 patients, it was not surprising that the CD16^{Int} cells had the lowest expression of CD44 indicating the highest potential for infiltration into the lung (FIG. 2e). Cluster analysis further confirmed these findings (FIG. 2f, blue circle). Since CD44^{Low} neutrophils are recruited to the lung to aid in clearance of bacterial pneumonia, we next investigated the phagocytic properties of the neutrophils from COVID-19 patients. FIG. 2g showed that CD 16^{Int} LDIB cells had a high uptake of pHrodo green S. aureus bioparticles suggesting a highly activated phenotype. One of the main ways that neutrophils eliminate pathogens is through NETs, the extravasation of DNA and protein to form a web like structure that can trap and kill extracellular pathogens. Increased NET formation from neutrophils in mouse models of bacterial sepsis increased platelet aggregation and coagulation. During analysis of the Wright-Giemsa stain for neutrophil morphologic characterization, we noticed that the LDIBs were spontaneously forming -NETs more prominently than CD16^{Neg} or CD16^{High} (FIG. 2h). Previous reports have also noted that low-density neutrophils readily form NETs causing endothelial vessel and organ damage in autoimmune phenotypes, which further confirms the pathogenic role of LDIBs in COVID-19.

Another neutrophil factor besides NETs that has been associated with driving platelet activation and thrombosis is CD40. Inhibition of the neutrophil-platelet CD40/CD40L axis with anti-CD40 antibody significantly diminished pulmonary edema, platelet activation and neutrophil recruitment to the lungs in a mouse model of transfusion related acute lung injury (TRALI). Assaying for CD40 expression on the neutrophil subsets, we found the overall neutrophil population in severe patients had a trending increased expression of CD40 as assessed by cluster analysis and flow cytometry (FIG. 3b, red circle) although not statistically significant. Strikingly, CD40 expression on the total neutrophils and CD16^{Int} LDIB population significantly positively correlated with severe COVID-19 patients' D-dimer and ferritin levels (FIG. 3c, d), suggesting a potential involvement of severe inflammation and thrombus formation.

### Clinical Significance of LDIB Neutrophils in CAC

Understanding the etiology of CAC is of paramount importance so that early adjustments in clinical management can be made to improve overall survival outcomes. Anti-coagulation therapy has been shown to increase the overall survival of both non-ventilator and ventilator dependent COVID-19 patients. However, anti-coagulation therapy comes with risk and is contraindicated in some patients. Therefore, it is necessary to delineate which patients are at the highest risk for thromboembolic complication and determine other potential strategies to mitigate inflammation induced coagulation in these patients.

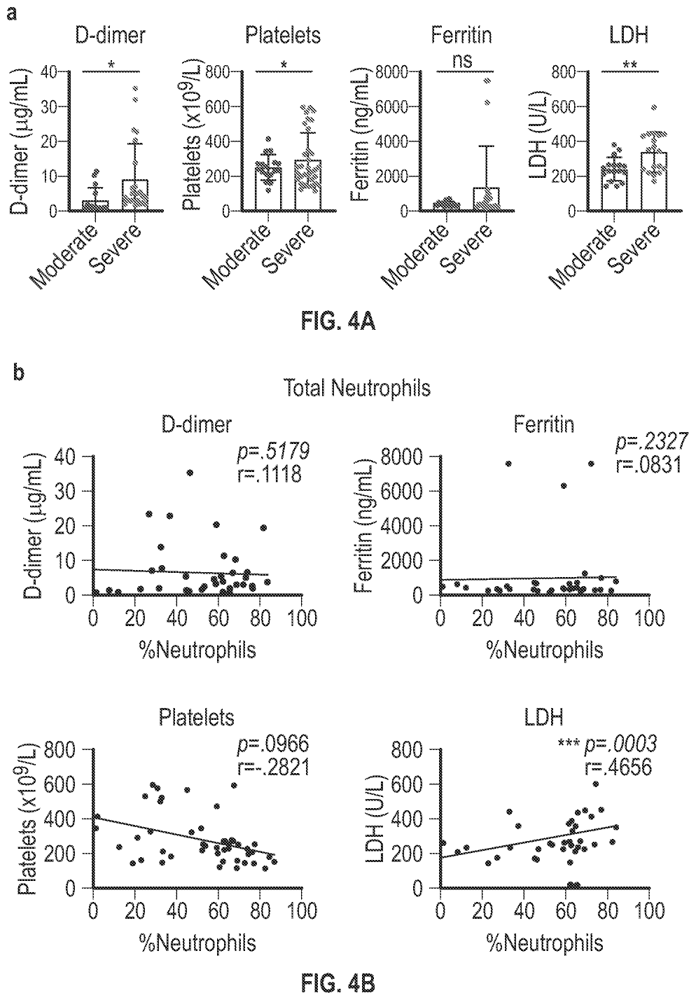

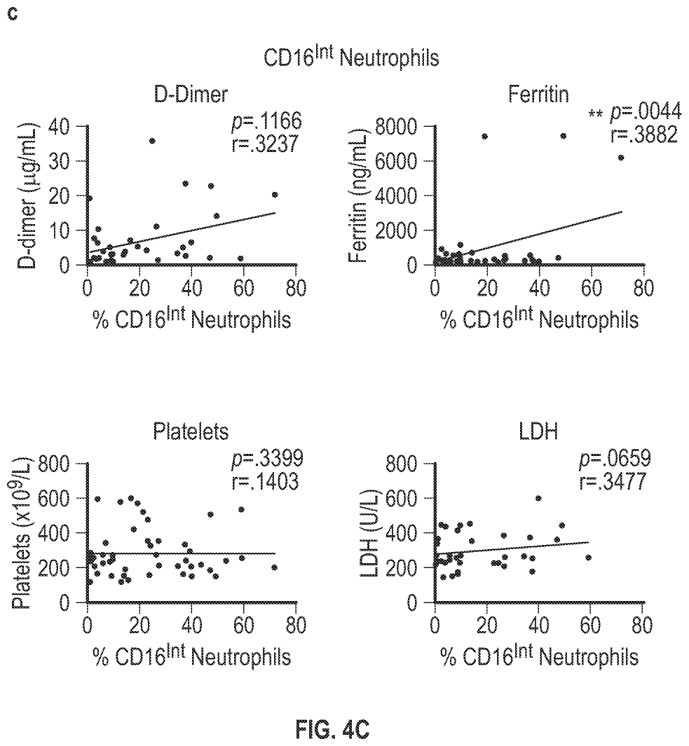

Two of the main clinical markers used to monitor coagulation state are D-dimer and platelet count, where increased D-dimer levels and decreased platelet counts are associated with coagulation. Looking into our COVID-19 cohort, we found that severe patients had an elevated level of D-dimer compared to moderate patients (FIG. 4a). The platelet levels were also increased in severe patients. Two other clinically important markers used to monitor systemic inflammation are ferritin and lactate dehydrogenase (LDH). While ferritin was not different between the two groups, it was elevated in moderate and severe COVID-19 patients as compared to the normal range. Increased LDH levels as seen in the severe cohort were often associated with more severe lung damage and tissue injury.

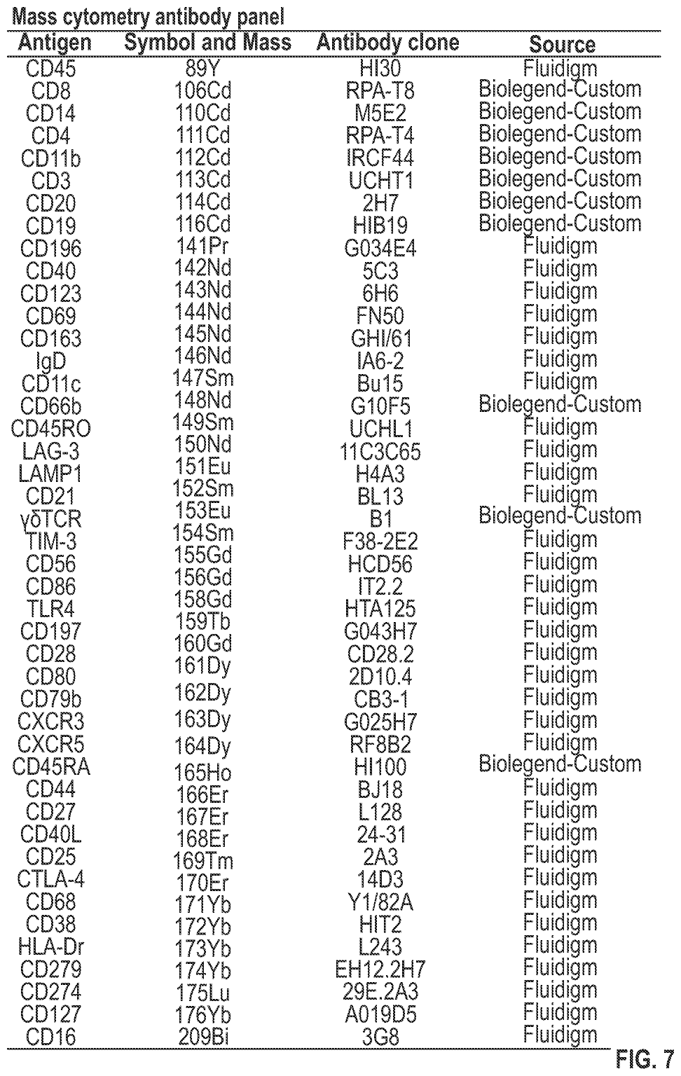

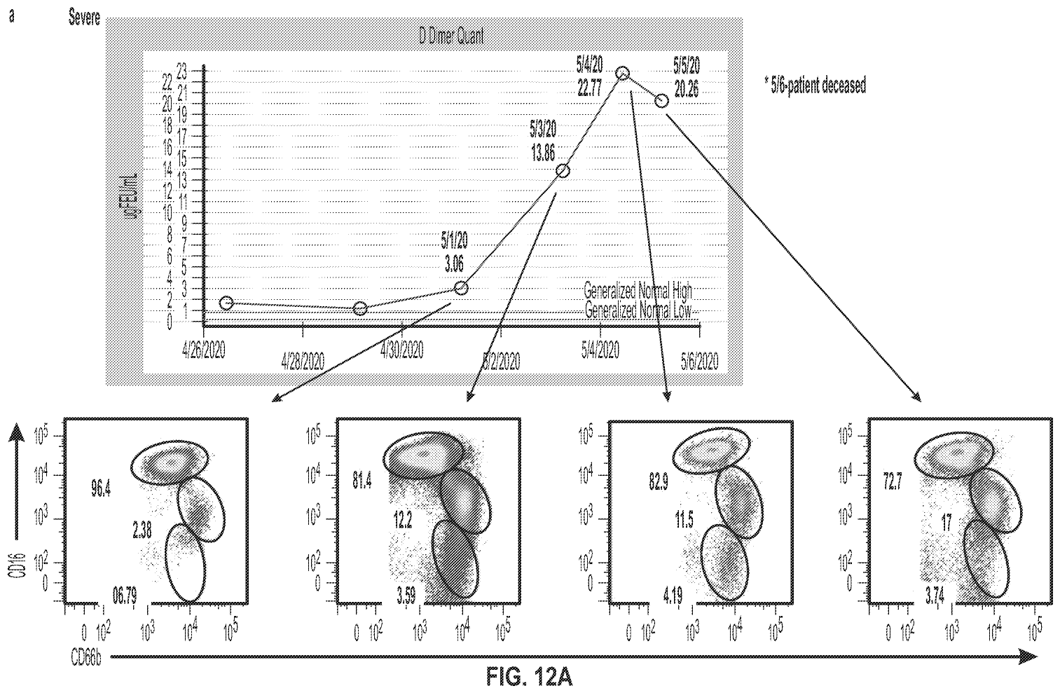

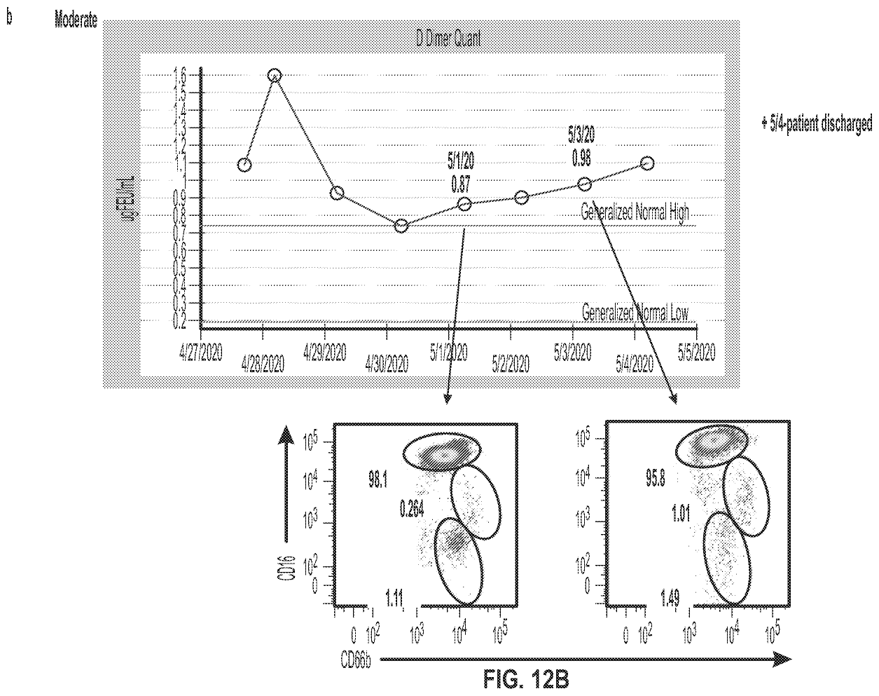

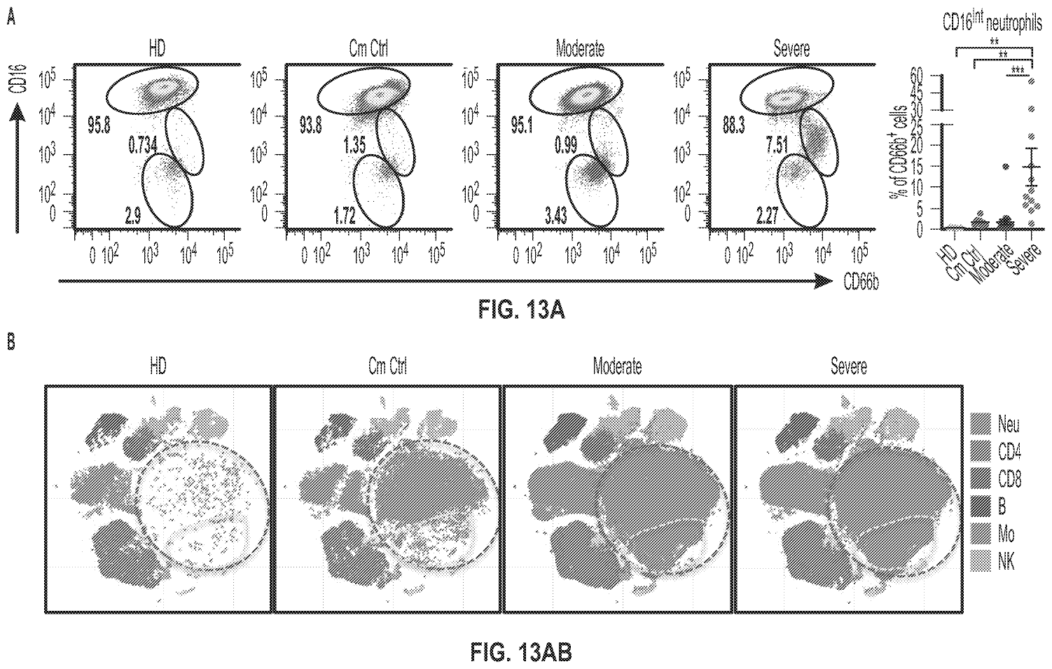

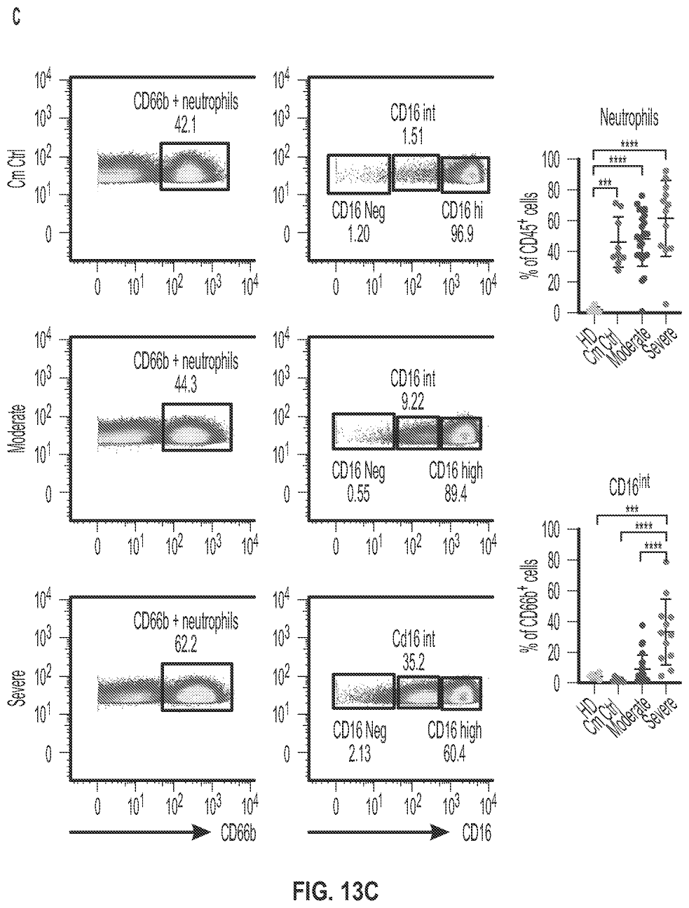

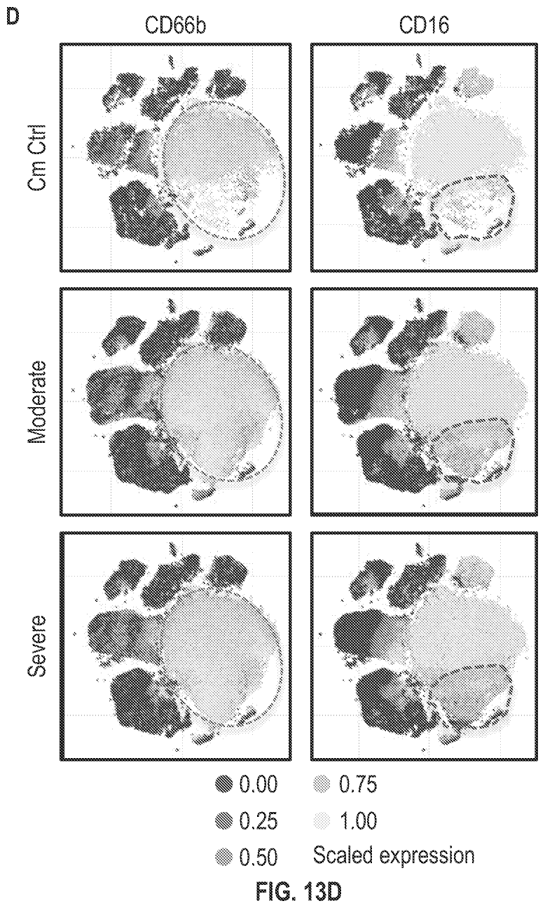

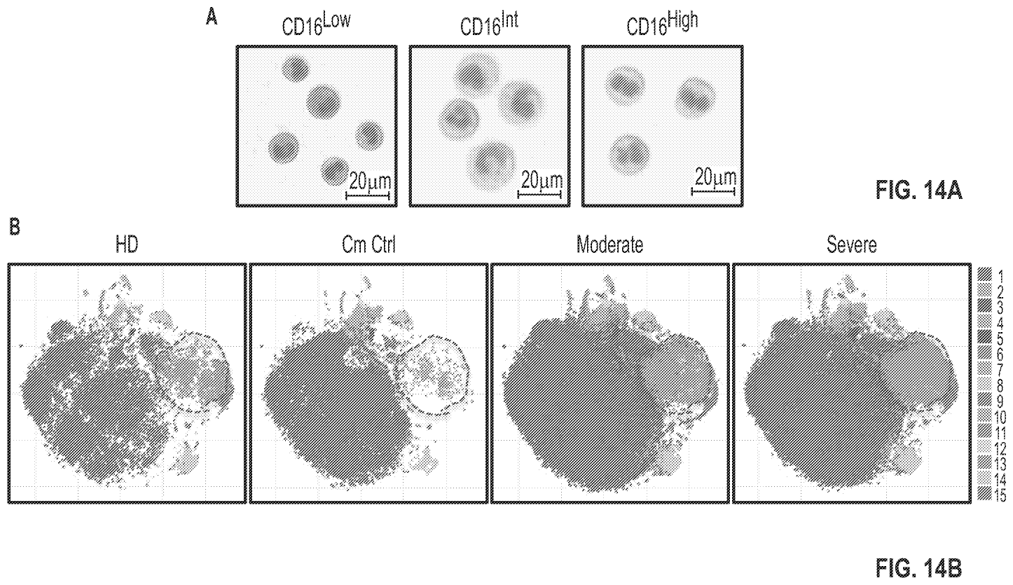

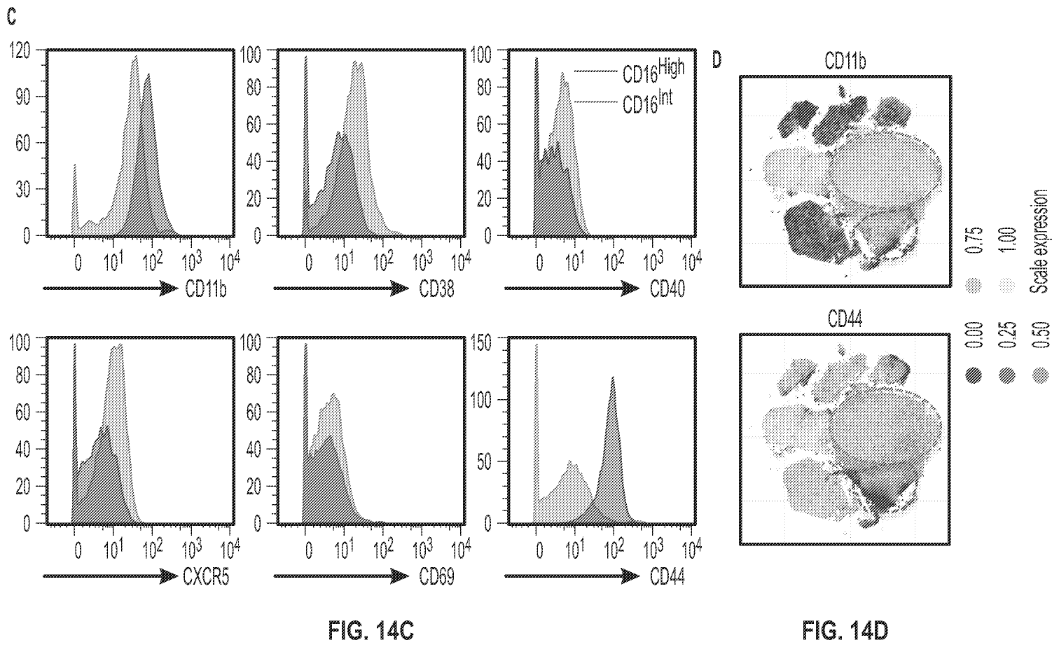

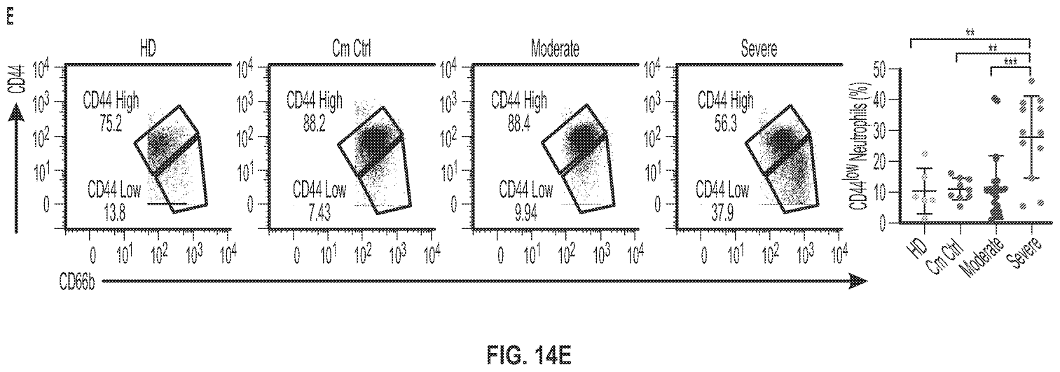

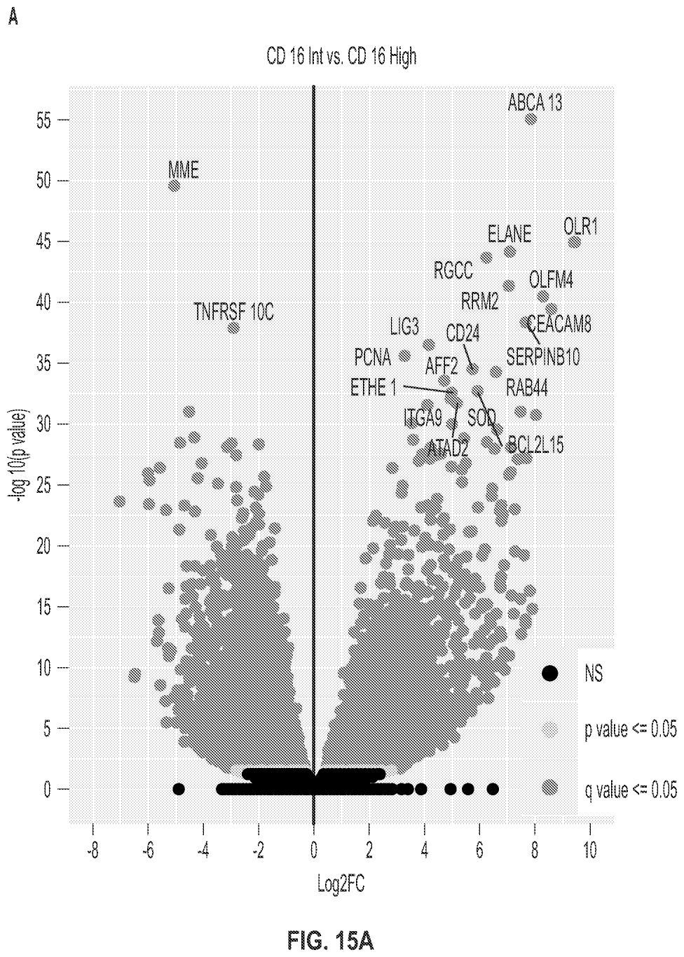

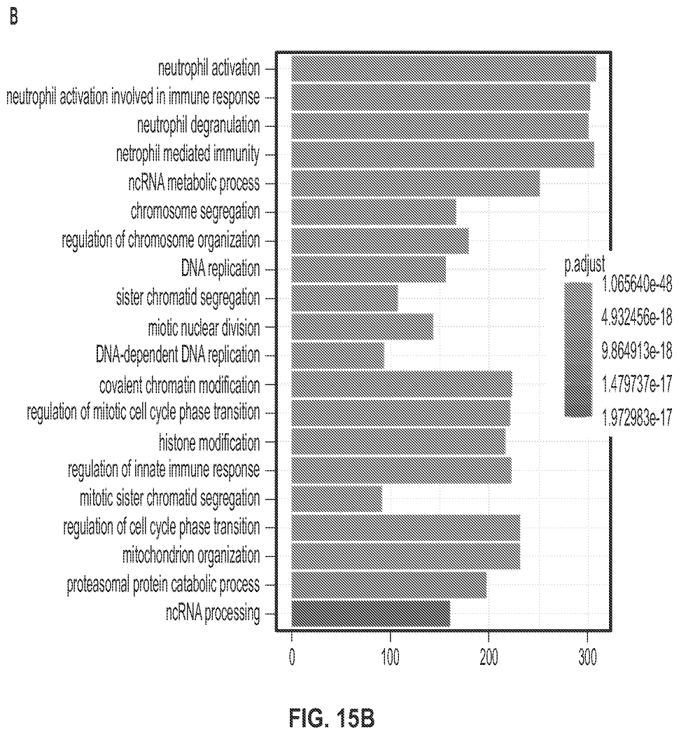

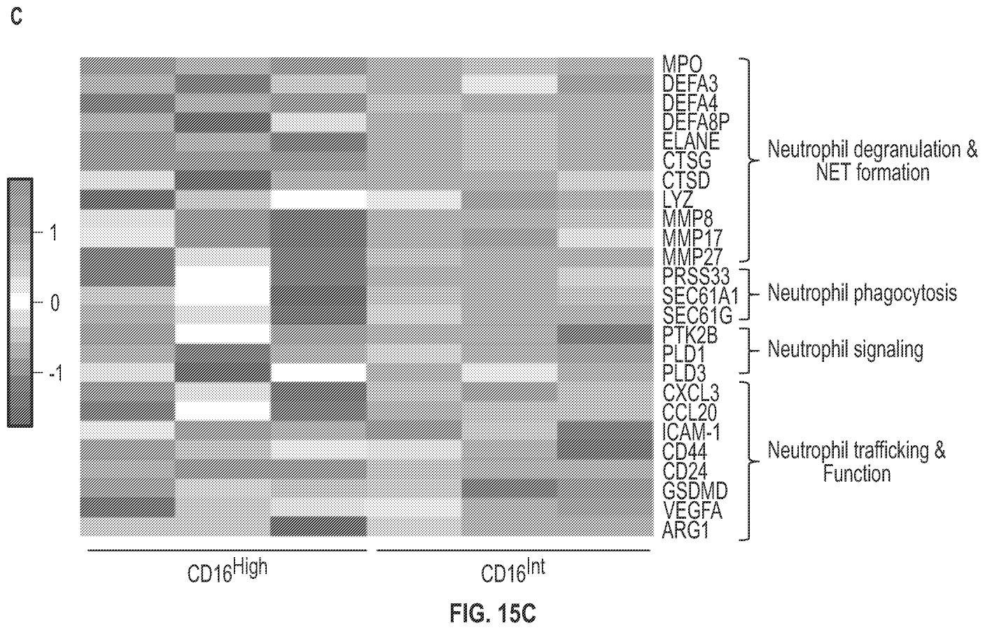

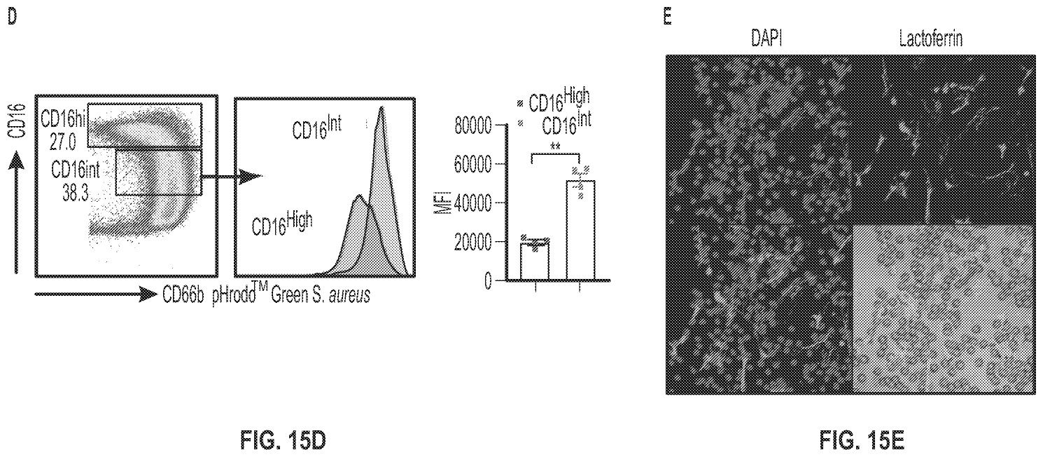

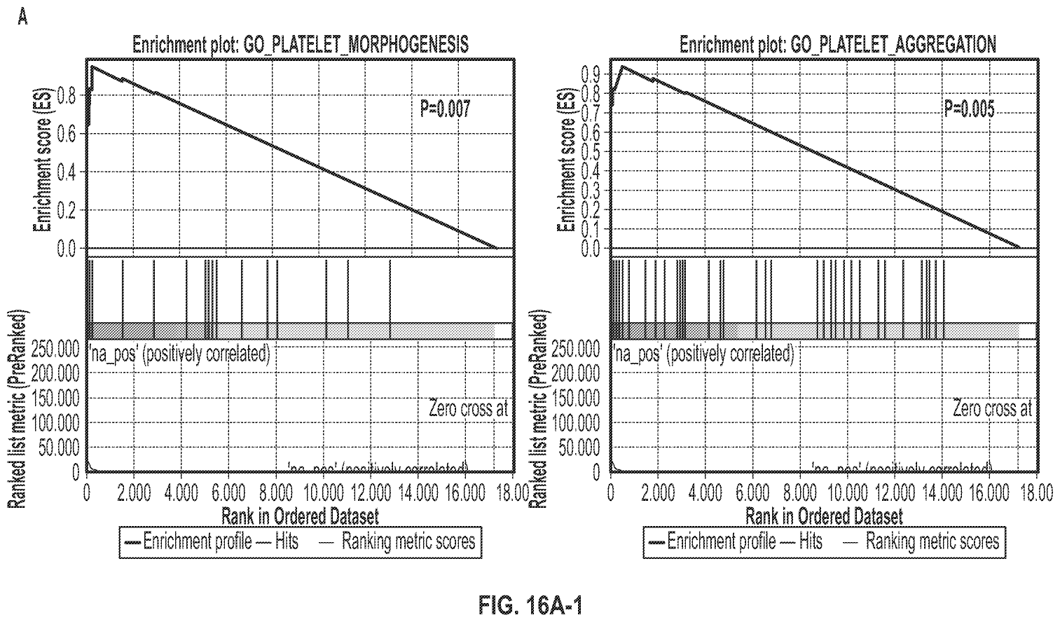

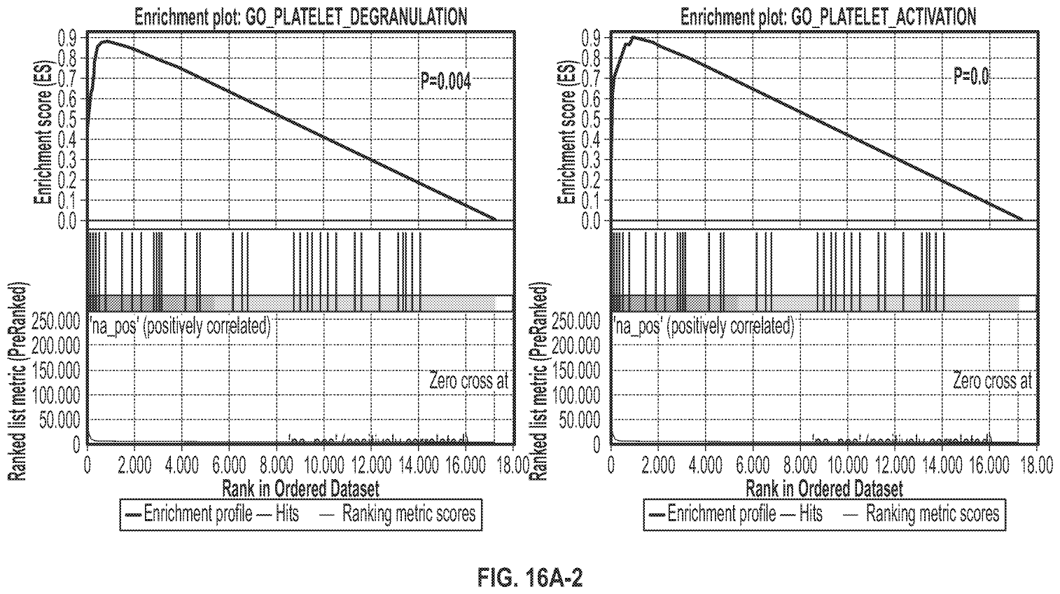

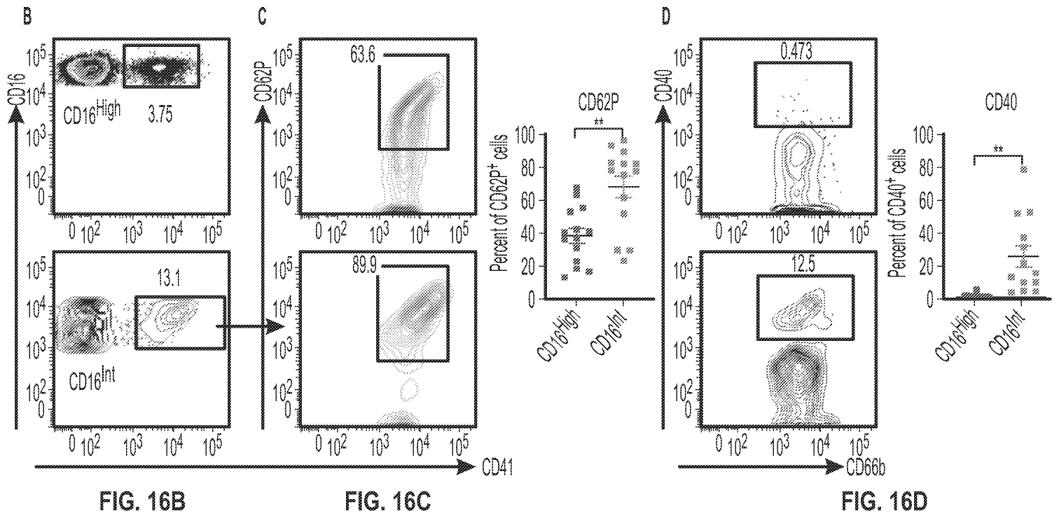

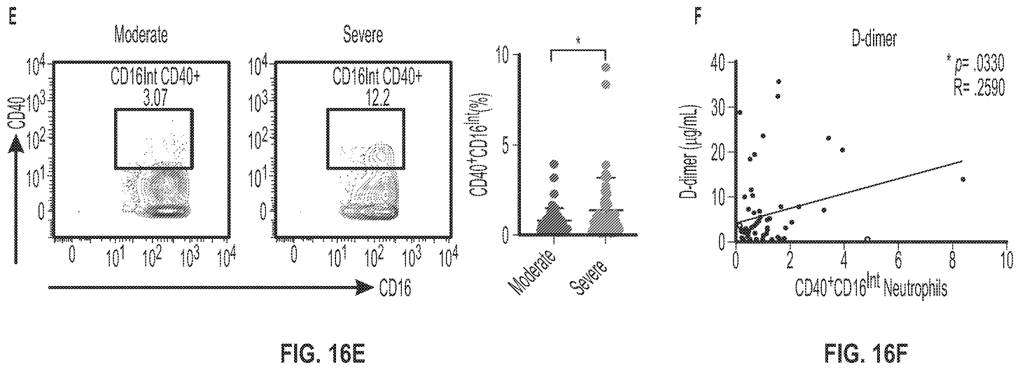

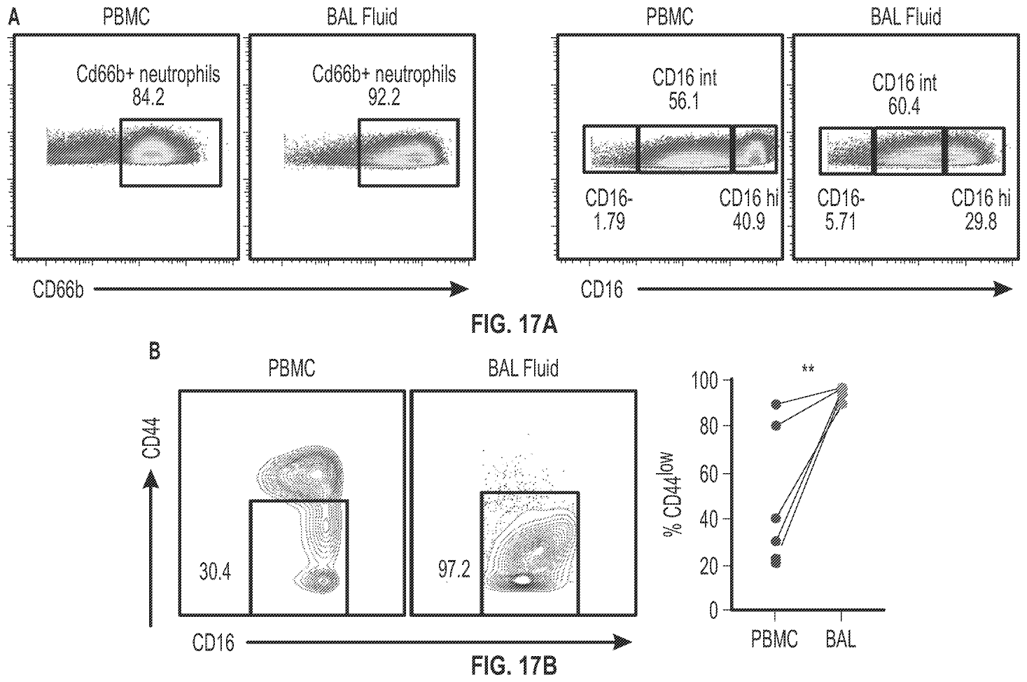

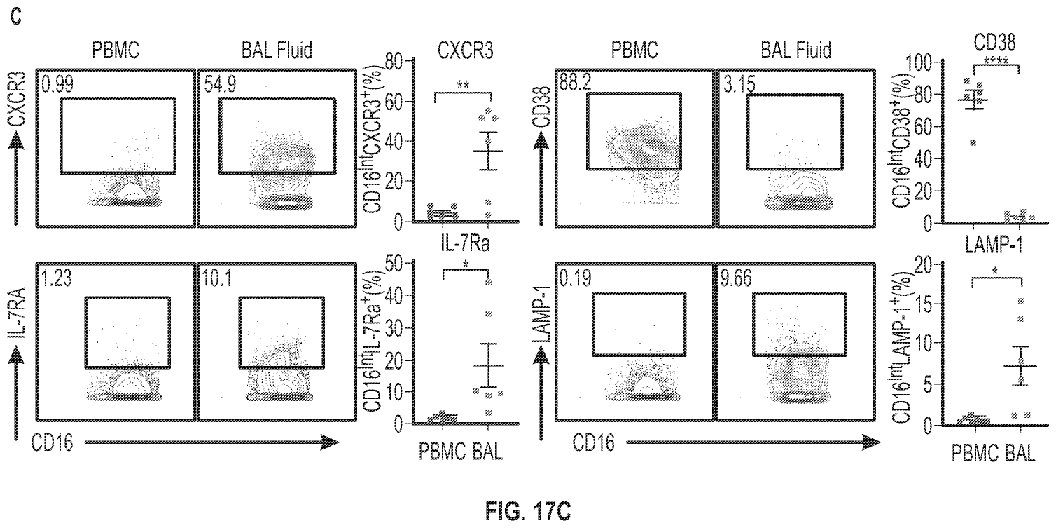

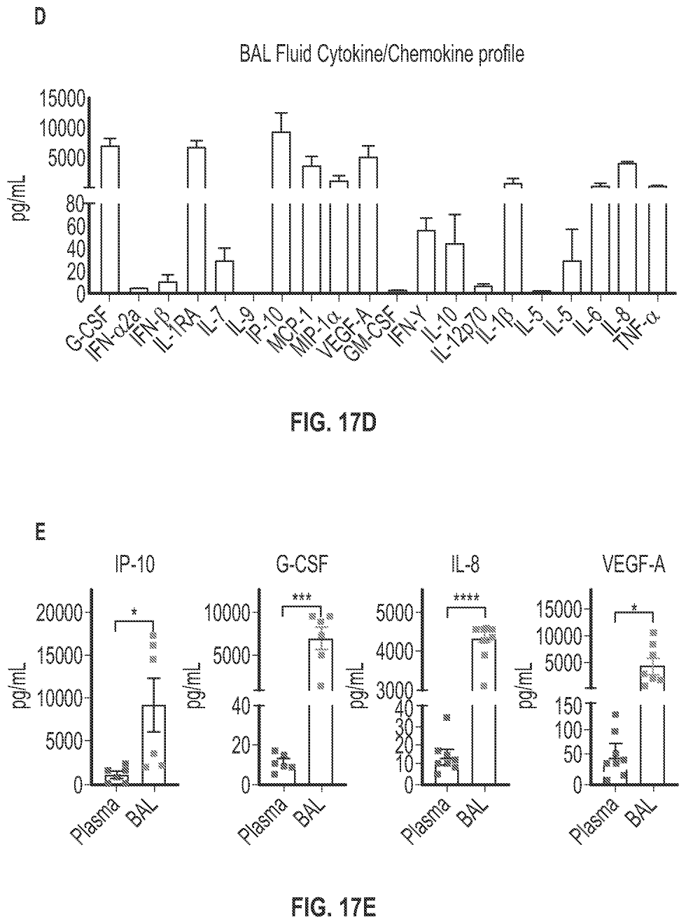

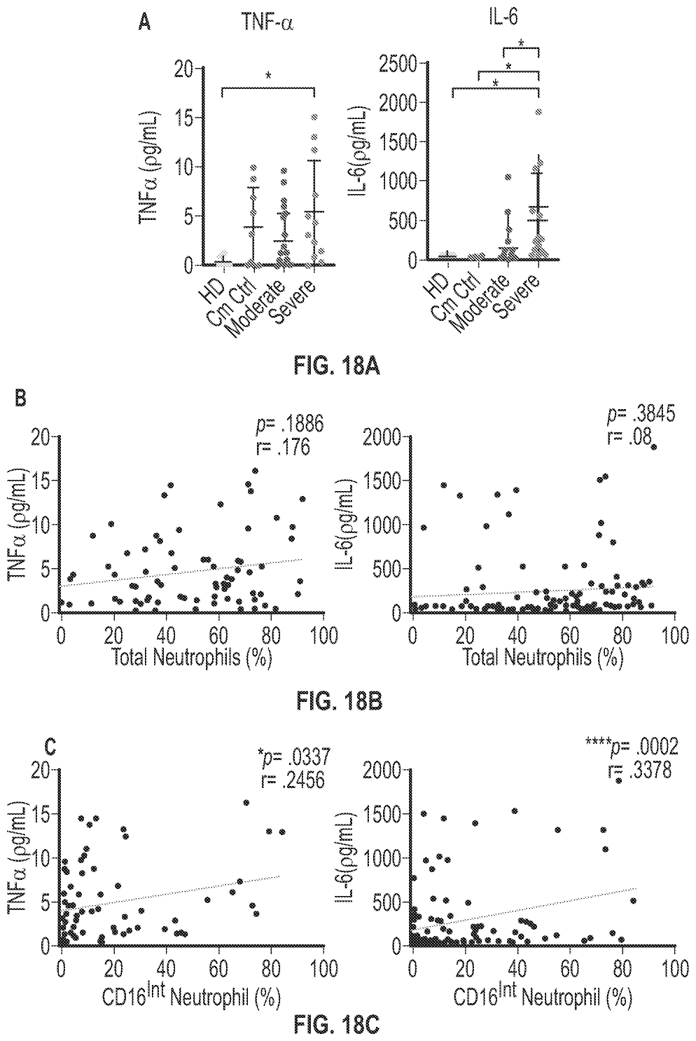

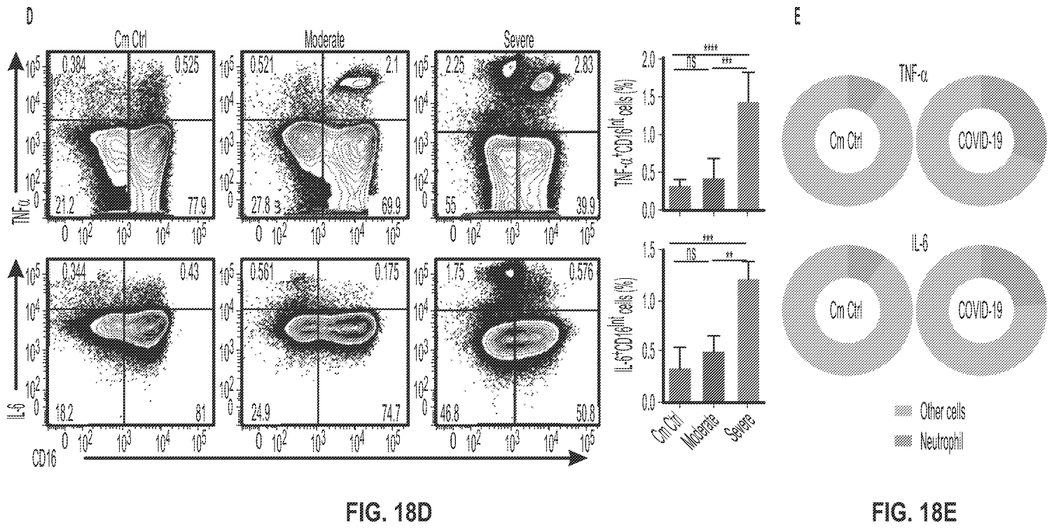

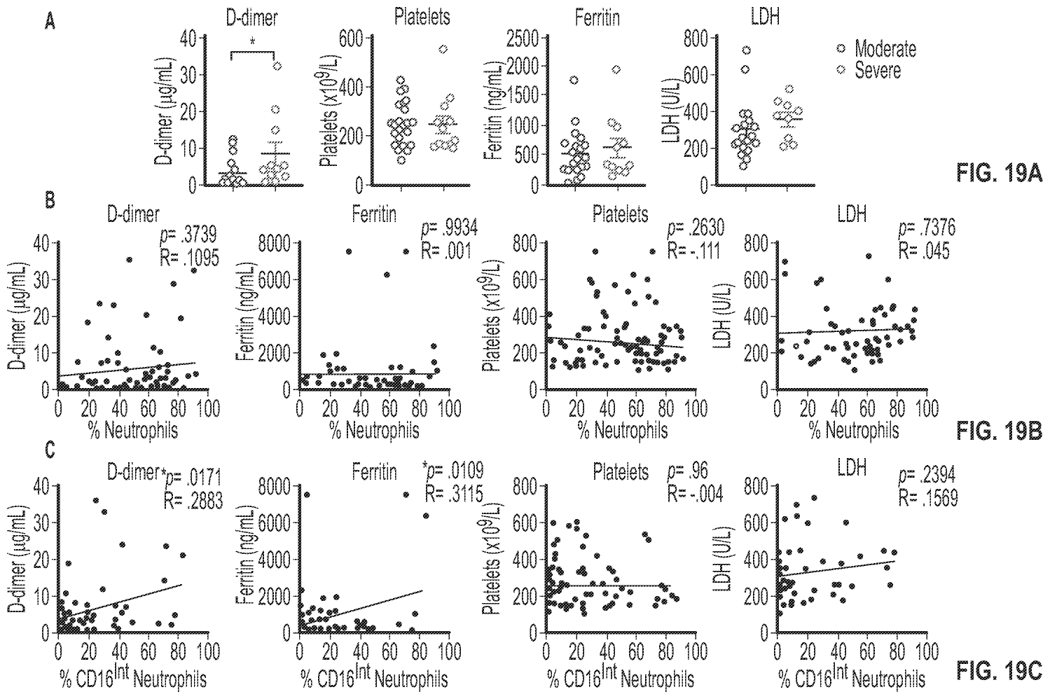

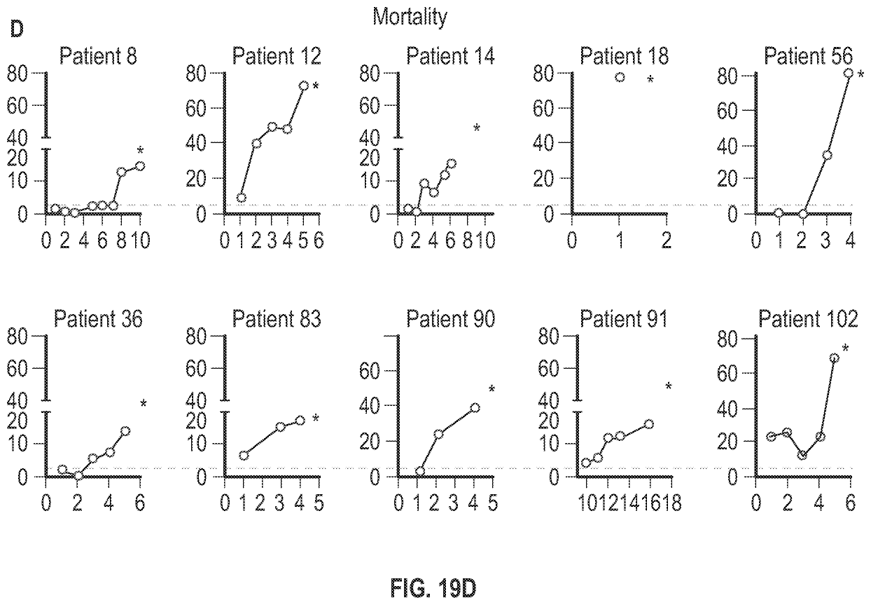

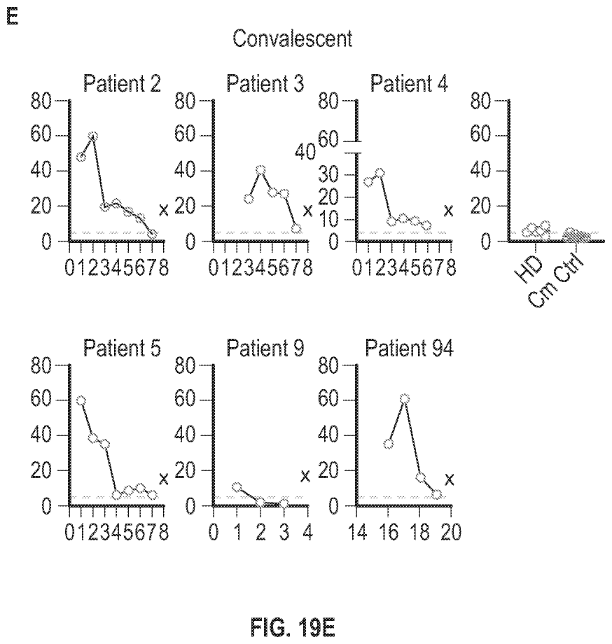

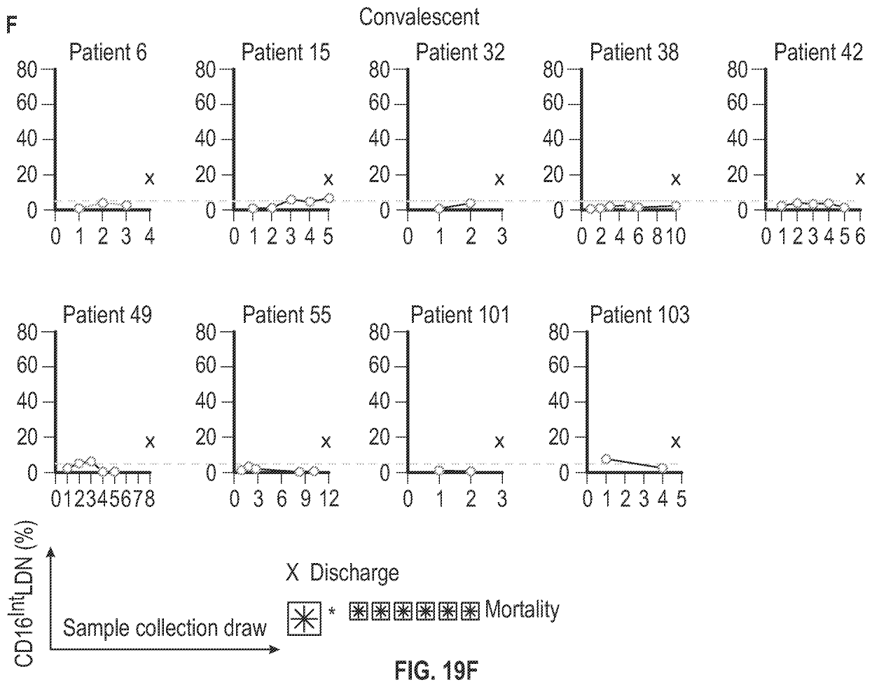

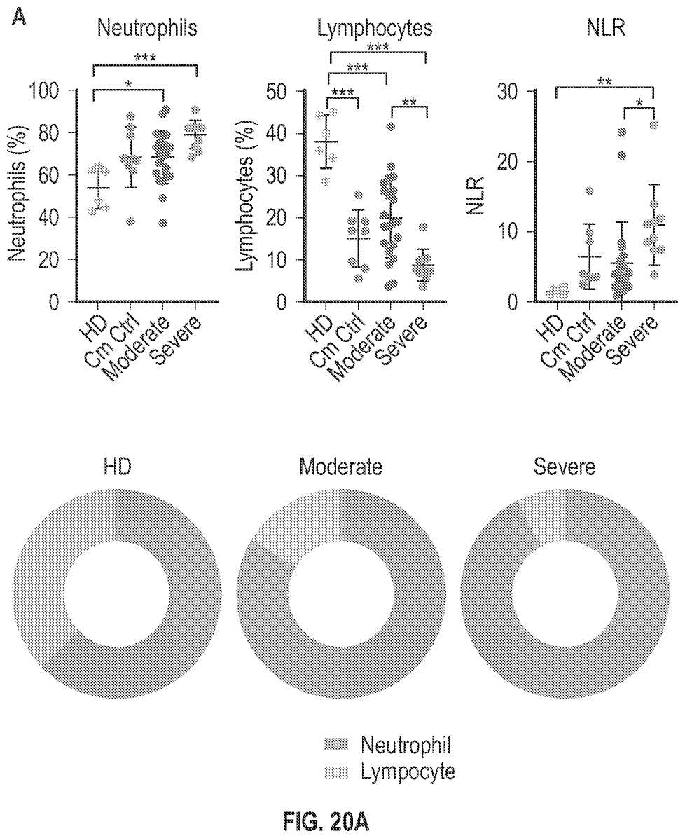

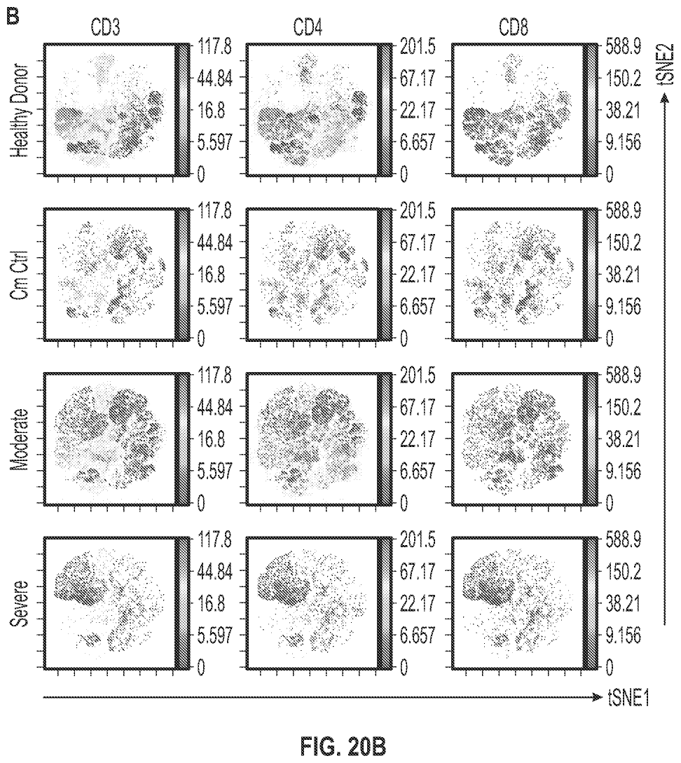

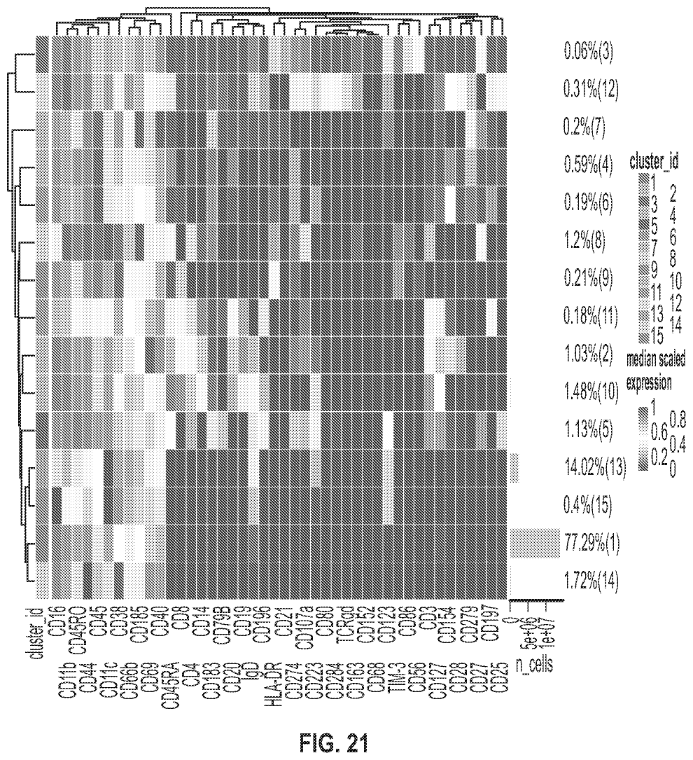

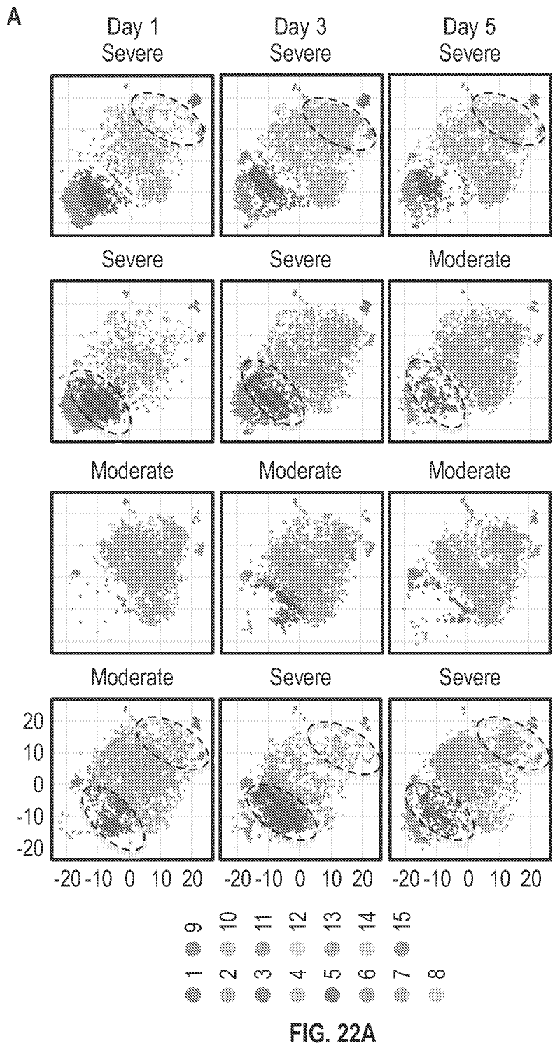

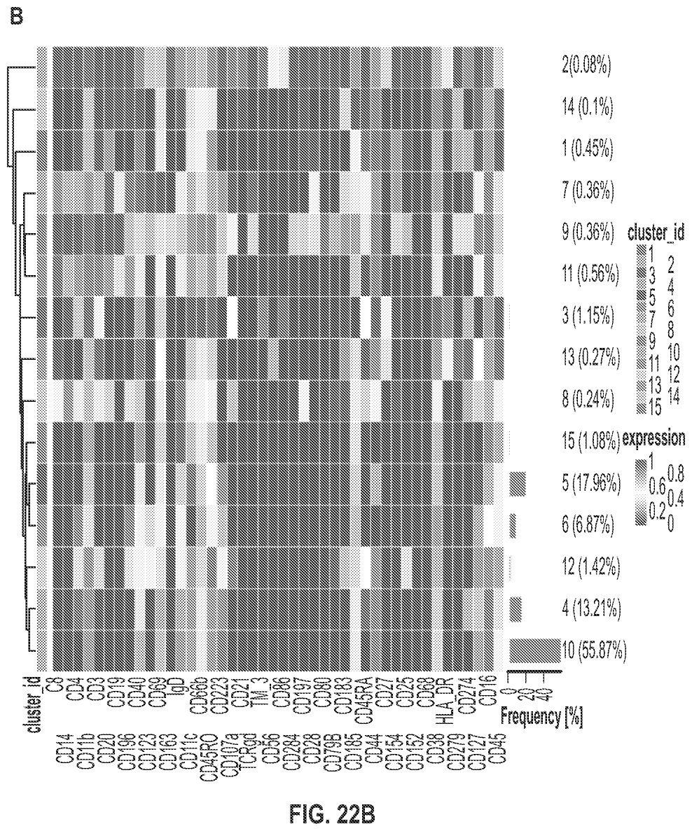

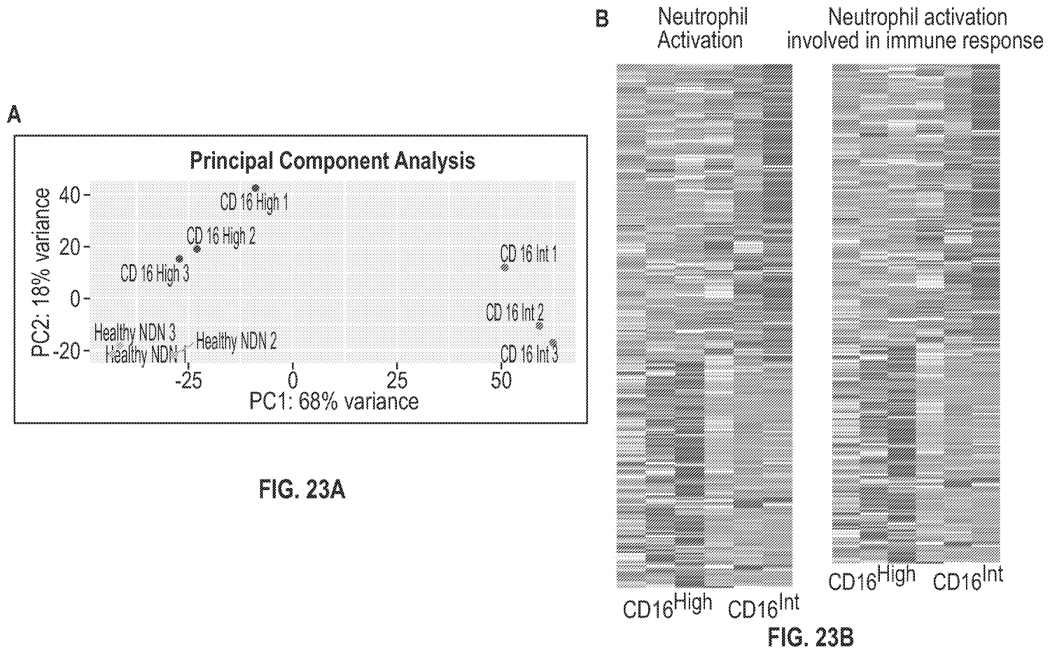

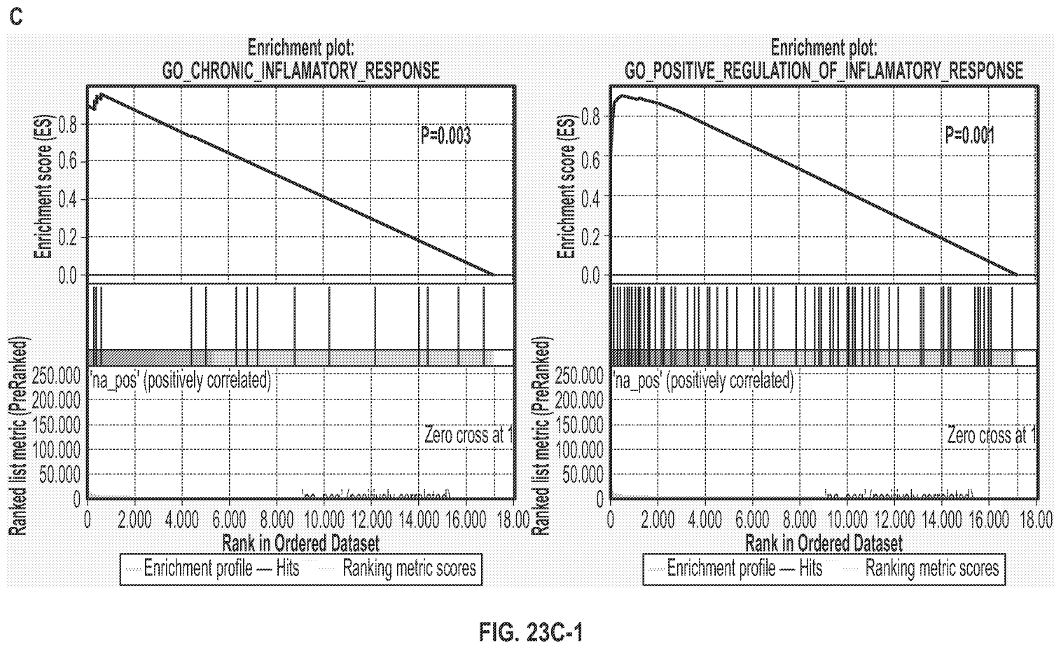

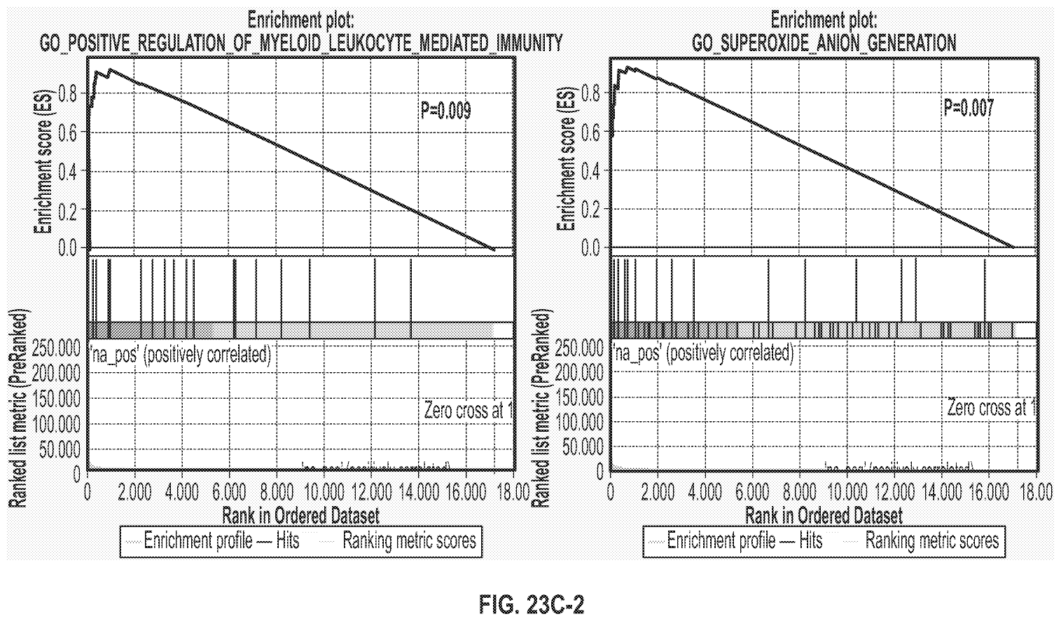

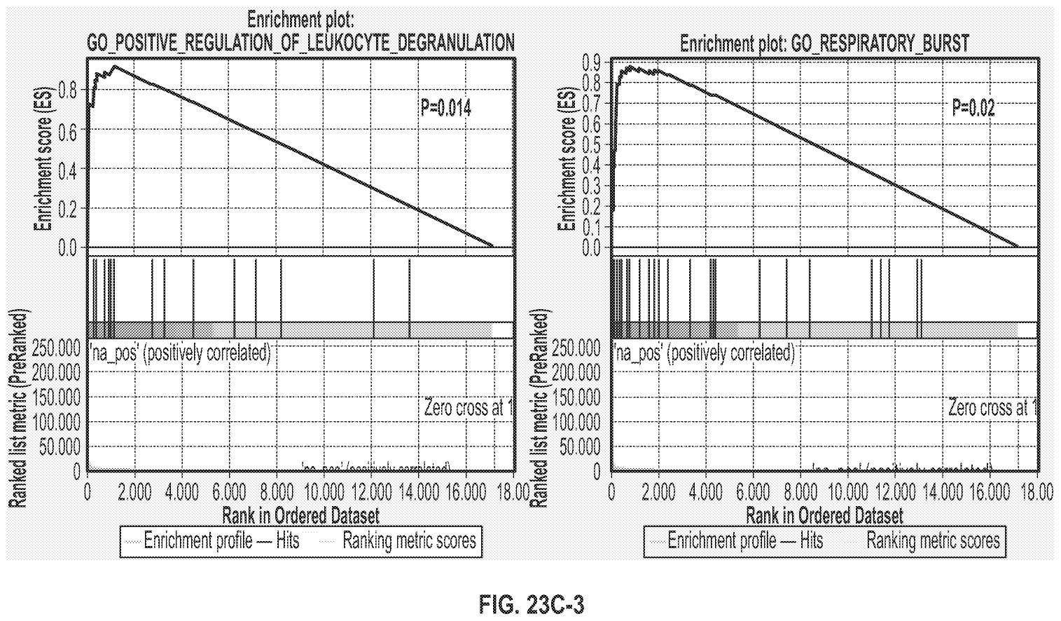

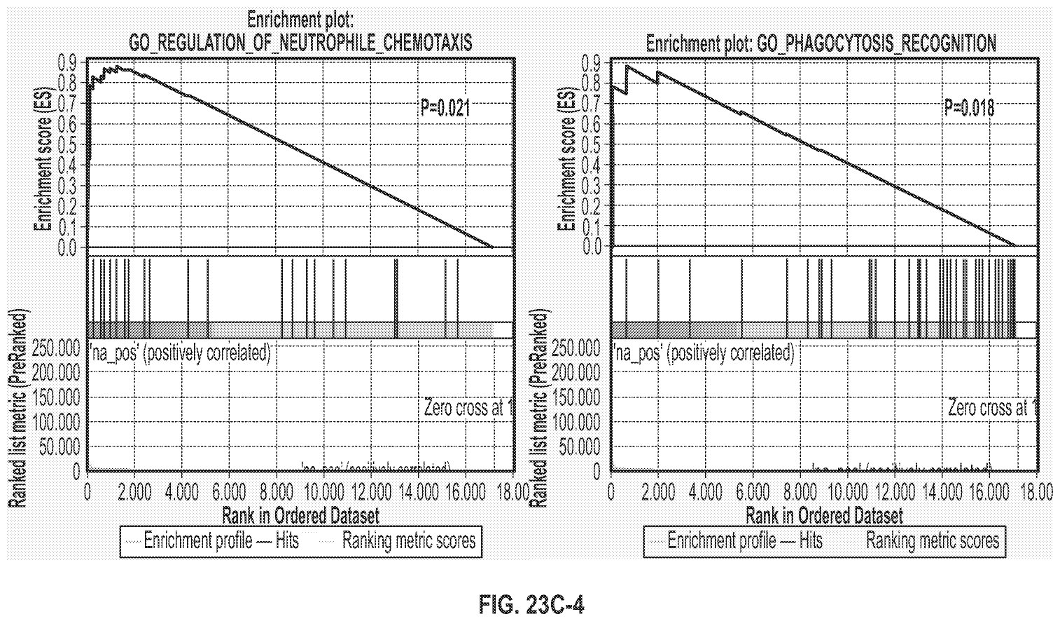

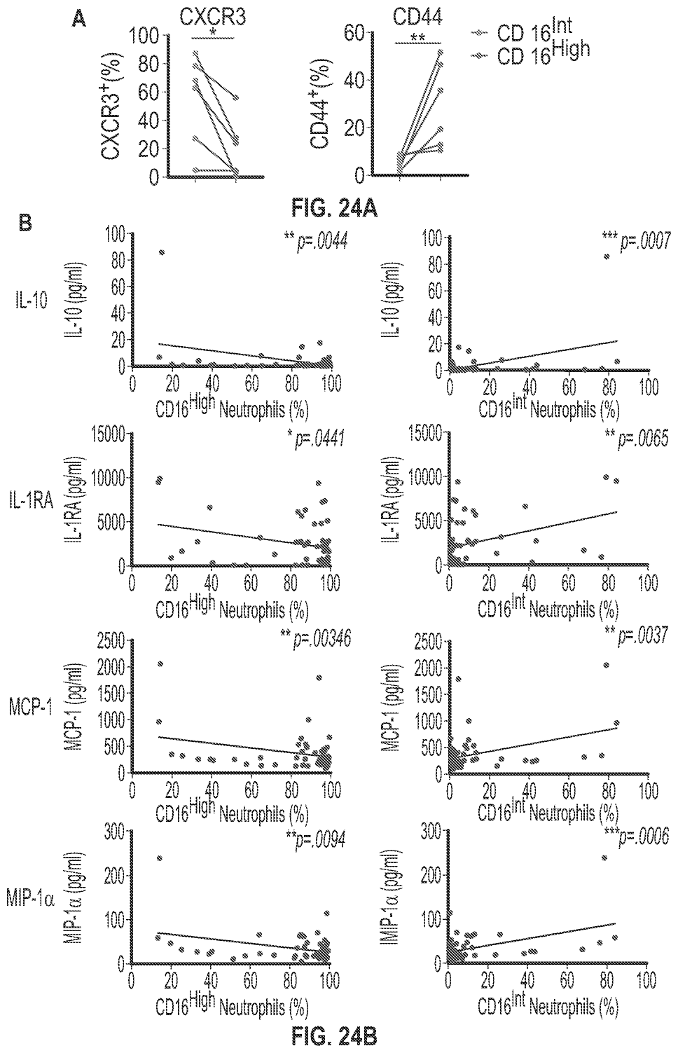

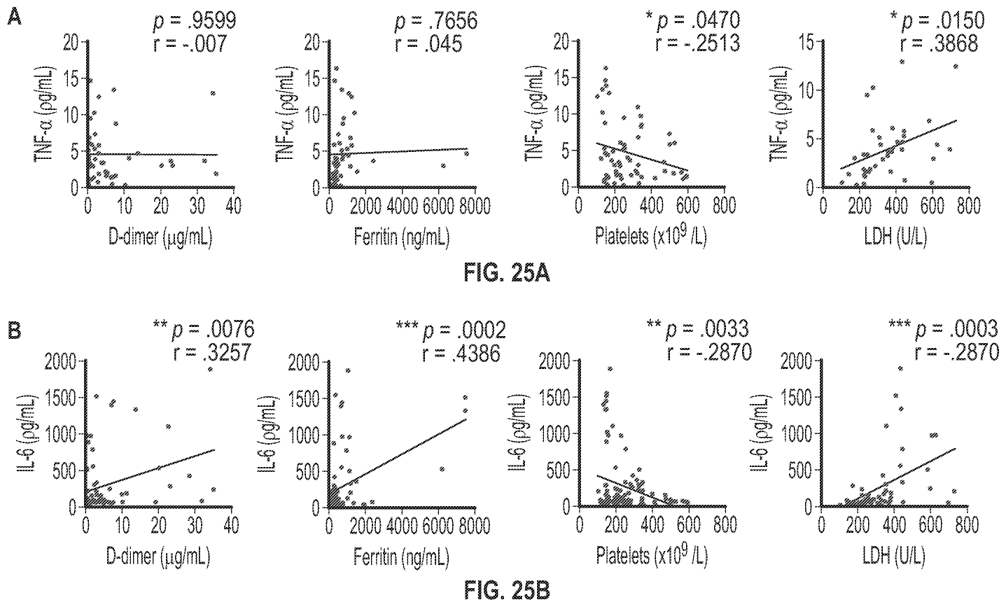

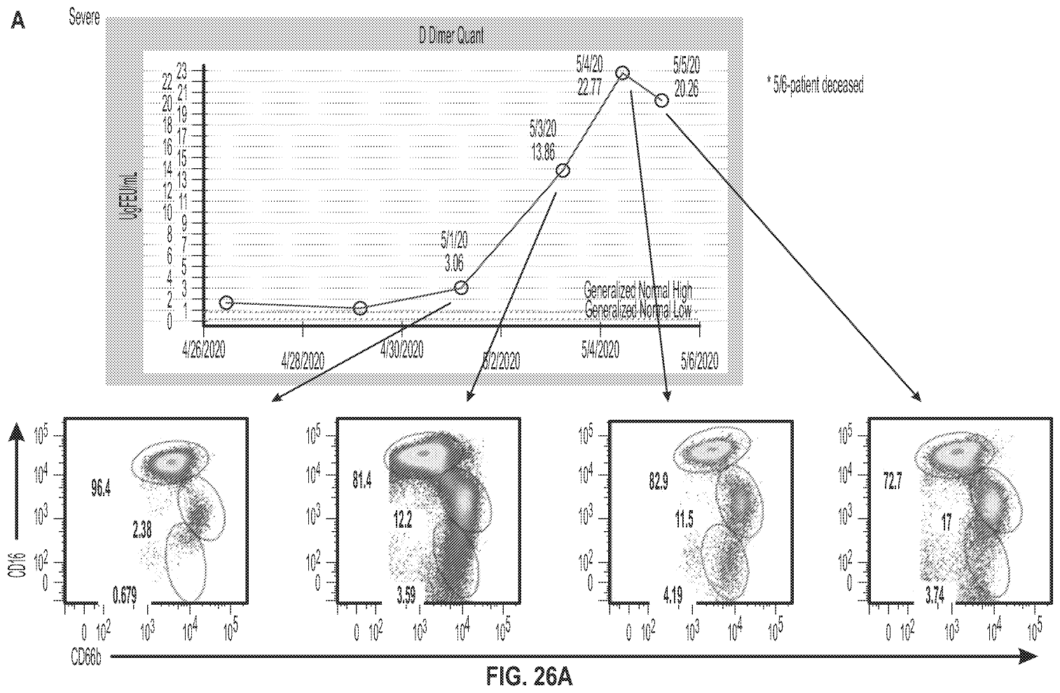

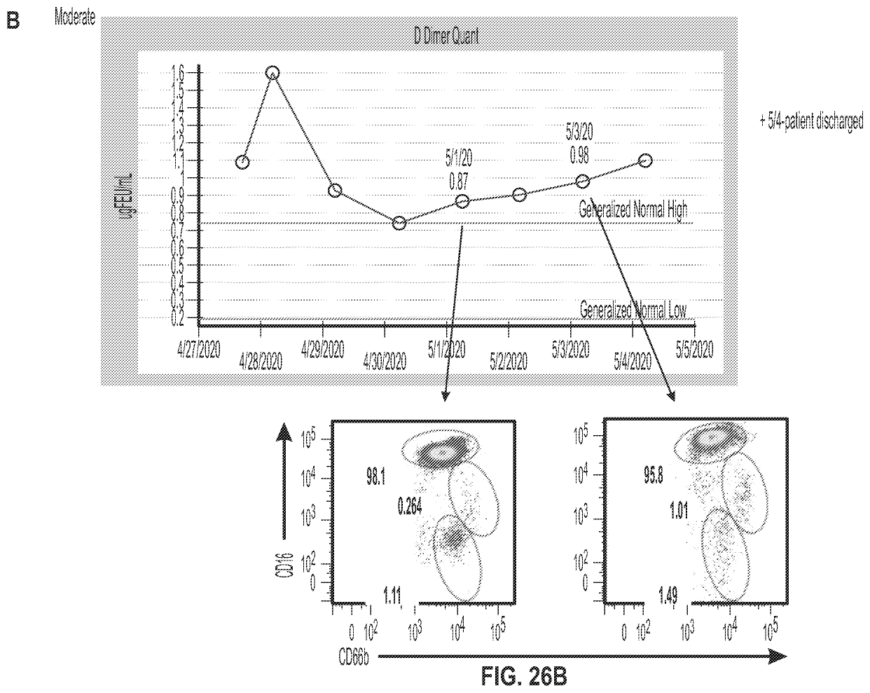

Having a better understanding clinically of the relevant markers in our two patient cohorts, we first sought to determine if overall neutrophil percentage was a good diagnostic tool to determine high risk of thromboembolic event. FIG. 4b showed that overall neutrophil percentage did not correlate with D-dimer or ferritin levels. However, overall neutrophil percentage did negatively trend with platelet counts and positively correlate with LDH levels suggesting some association with thrombosis and declining status. Conversely, the CD16^{Int} population significantly correlated with ferritin but not platelets or LDH (FIG. 3c). For correlation with D-dimer, we clearly saw a trend between the LDIB population and D-dimer, although statistical significance was not reached (FIG. 4c). Two issues related to this analysis were that D-dimer level was not measured frequently in our cohort of patients, particularly for moderate patients and all patients received anti-coagulation therapy (FIG. 7). However, despite this, trending serial analyses of individual patients' LDIB populations with D-dimer demonstrated appreciable associations and a pronounced phenotype. Taking patient 12 as a representative severe patient, there was a clear correlation between their rising D-dimer levels and increasing LDIB population leading up to their death (FIG. 12a). Alternatively, in patient 9 from the moderate group, both their D-dimer and LDIB percentage were only marginally elevated prior to discharge (FIG. 12b). Therefore, taking the statistical and descriptive data together our finding suggests that the LDIB percentage rather than overall neutrophil percentage correlates better with coagulation status in COVID-19 patients.

### Contribution of LDIBs to Cytokine-Mediated Coagulopathy in COVID-19 Patients

It has been established that severe COVID-19 patients have elevated levels of pro-inflammatory cytokines resulting in cytokine storm. Two of the main cytokines that have been found to be consistently elevated among the most severe COVID-19 patients are TNF-α and IL-6. In cytokine storm, TNF-α causes vasodilation and increases vascular permeability to allow for immune infiltration, resulting in pulmonary edema. IL-6 induces a multitude of immunomodulatory functions including T cell and B cell activation, acute phase reactive protein production from the liver, and platelet hyper-activation. Both IL-6 and TNF-α have been reported to promote coagulation through activation of the extrinsic coagulation cascade by inducing endothelial expression of tissue factor. Therefore, given the associations between IL-6 and TNF-α with cytokine storm and coagulation, we wanted to determine if LDIBs and/or overall neutrophils were contributing to the generation of these cytokines and whether they correlated with clinical markers of coagulation.

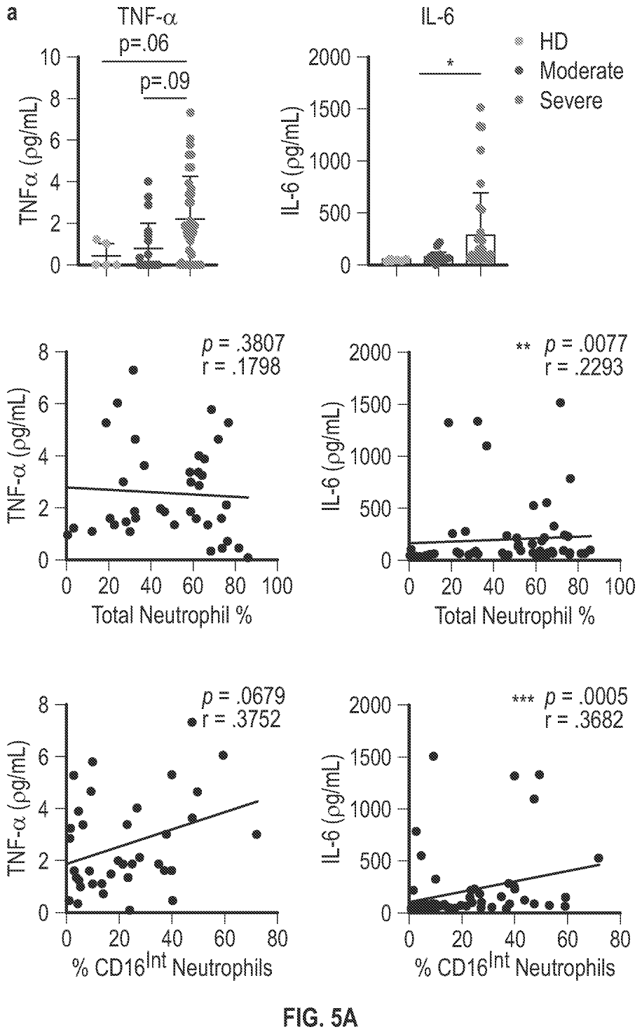

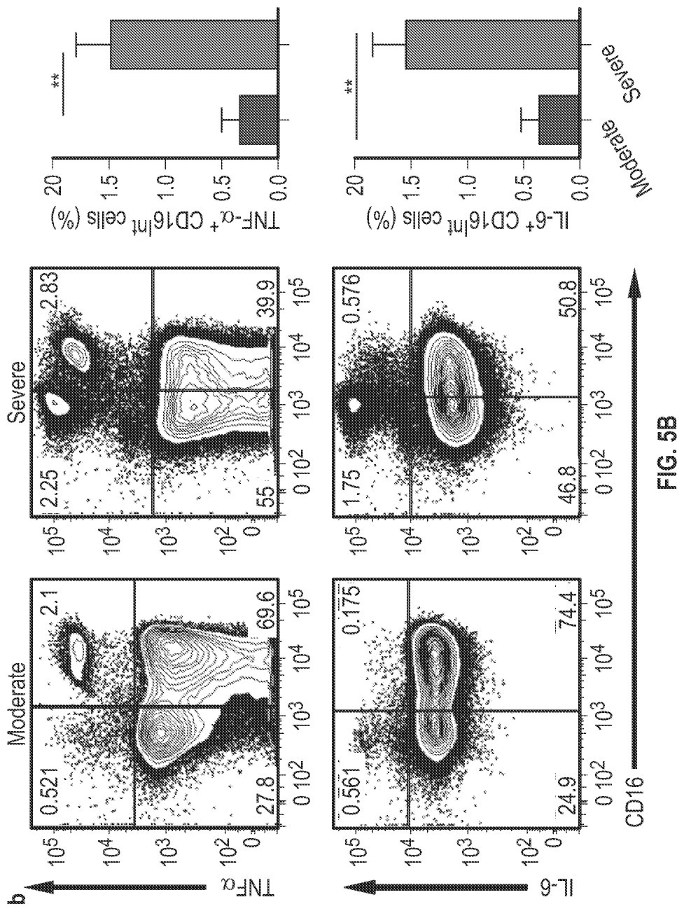

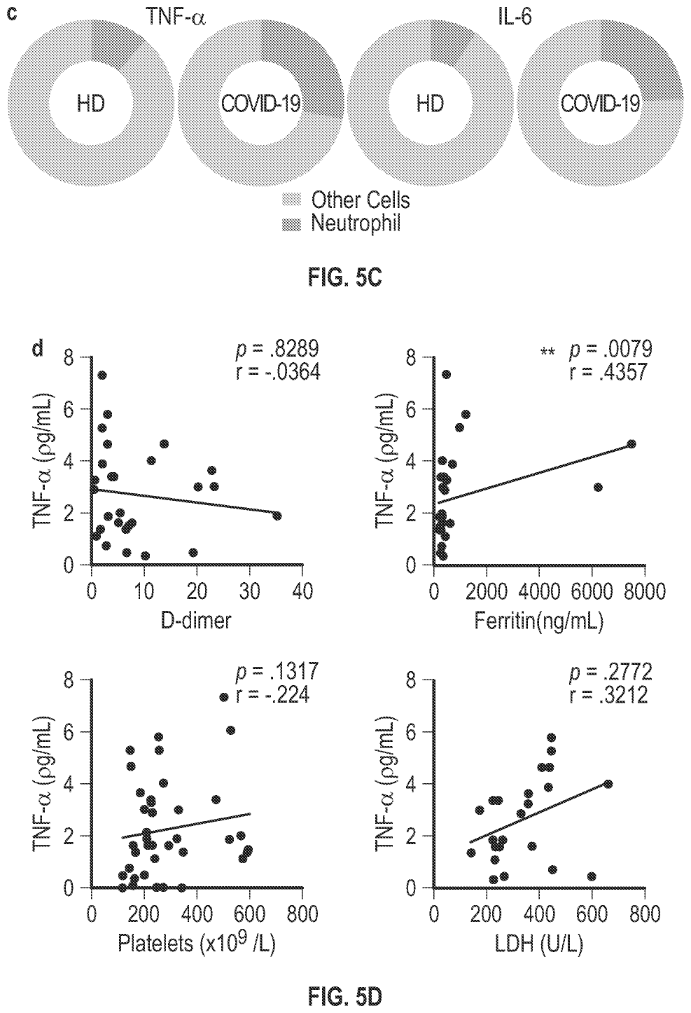

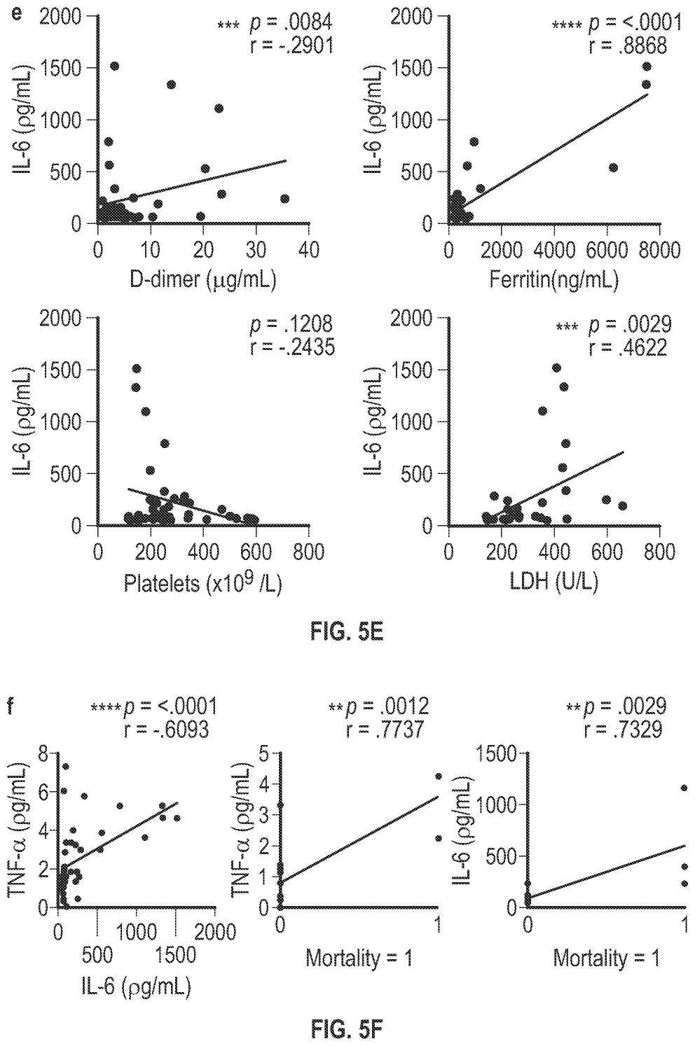

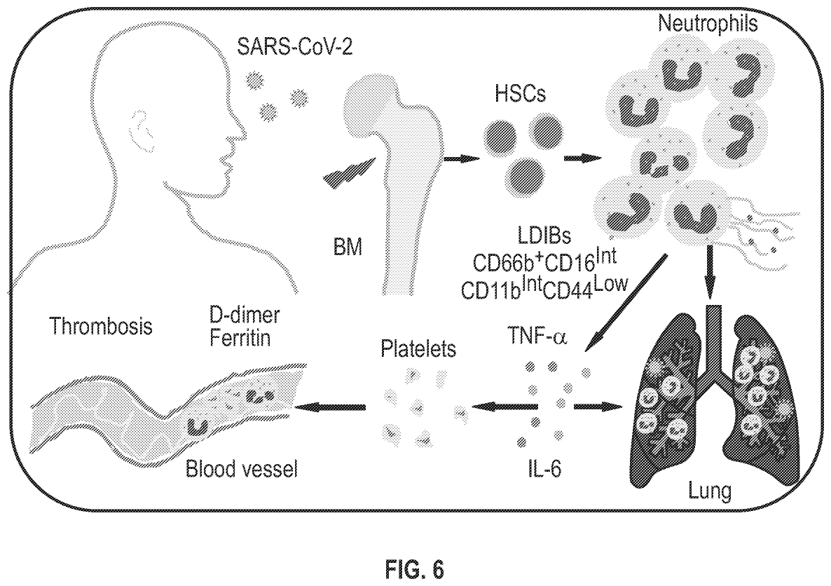

We first measured plasma concentrations of TNF-α and IL-6 in the serial blood samples of patients compared to healthy donors (FIG. 5a). The overall plasma level of TNF-α was low but was elevated in the severe group compared to moderate and healthy donors. IL-6 showed significant increases above moderate patients. Correlating the plasma level of TNF-α with overall neutrophil percentage showed no significant association while IL-6 level was significantly correlated with overall neutrophil percentage (FIG. 5b). Furthermore, the CD16^{Int} LDIB population showed a positive significant correlation with IL-6 levels across all patients and donors and TNF-α level showed a strong trend with LDIB frequency. These results further emphasize the particular pro-inflammatory characteristics of LDIBs as compared to overall neutrophils.

We next sought to examine whether neutrophils directly contribute to these systemic cytokine pools. Stimulation of whole blood samples with LPS showed LDIBs in the severe patients were capable of producing significant amounts of TNF-α and IL-6 compared to moderate patients (FIG. 5b). In addition, neutrophils from all COVID-19 patients increased their proportion of total cytokine-producing cells compared to those from healthy donors (FIG. 5c). Further investigation into the correlation of TNF-α levels with other clinical markers of inflammation demonstrated a significant correlation with ferritin but no correlation with D-dimer, platelets and LDH (FIG. 5d). In contrast, IL-6 levels were positively correlated with the levels of D-dimer, ferritin and LDH and negatively trending with platelets (FIG. 5e). Furthermore, these two cytokines correlated with each other and both TNF-α and IL-6 significantly correlated with patient mortality (FIG. 51). Overall, these data suggest that neutrophils, particularly the CD16^{Int} LDIB subset, are substantial contributors to the cytokine storm seen in COVID-19 patients. In patients with severe elevations in LDIBs or “LDIB crisis”, the dramatic increase in production of TNF-α and IL-6 likely causes a profound upregulation of tissue factor resulting in thrombus formation and D-dimer elevation,

### DISCUSSION

Our study aimed to investigate the etiology of CAC in an effort to help guide patient management and improve survival outcomes. On average, approximately one third of critically ill COVID-19 patients develop CAC and thromboembolic complications during the course of the disease. The most common primary outcomes are venous thromboembolism, ischemic stroke, myocardial infarction, and disseminated intravascular coagulation. In our own patient cohort, 8/13 (61.5%) of COVID-19 patients experienced a thromboembolic complication. Clinically, the majority of severe COVID-19 patients present with grossly elevated D-dimers. Treating high risk patients with a full dose of systemic anti-coagulation has been shown to he associated with a decreased risk in mortality. However, systemic anti-coagulation poses potential bleeding risks and is contra-indicated in some patients, especially those with numerous co-morbidities, which make up a significant portion of COVID-19 patients. Additionally, treating the coagulopathy targets the symptoms rather than the cause of the problem.

It has been proposed that the strong inflammatory response to COVID-19 is associated with CAC. One case study found that IL-6 levels significantly correlated with fibrinogen levels in mechanically ventilated COVID-19 patients. However, while this suggests that the unchecked inflammatory response could be contributing to CAC, the specific cellular etiology and mechanism have not been directly elucidated. One of the most notable immune disturbances in severe COVID-19 is neutrophilia and increased NLR. Both increased D-dimer and NLR have been associated with poor clinical outcomes. Therefore, we examined the possibility that the neutrophils are significantly contributing to the coagulopathy and could be used as an adjunct clinical measure to determine thromboembolic complication risk and guide treatment measures.

In agreement with previous reports, we found that severe COVID-19 patients have an increased neutrophil percentage and increased NLR. Here, we further detail the emergence of a novel immature neutrophil population, LDIBs, in the peripheral blood of the severe COVID-19 patients. These cells are identified by their distinct band shaped nucleus in addition to intermediate expression of CD11b and CD16, low expression of CD44 and high expression of CD40 (CD16^{Int}CD44^{Low}CD11b^{Int}). Like low-density neutrophils described in other inflammatory immune conditions, we were able to isolate these cells vial PBMC Ficoll pull down in COVID-19 patients but not in healthy donors. In accordance with previous reports, these cells readily make NETS which we captured via Wright Giemsa staining. In addition, CD40⁺ LDIBs correlate strongly with plasma levels of D-dimer and ferritin in severe COVID-19 patients. Overall, the combination of NET formation and CD40 expression indicates a neutrophil that is capable of promoting coagulation and thrombosis from CD40 mediated platelet activation and NET induced endothelial damage. Additionally, the down regulation of CD44 enables these cells to traffic to the lung where multiple published case studies demonstrate marked neutrophil infiltration into the lung tissue and subsequent damage. Neutrophil infiltration of the lung is accompanied by lung edema, endothelial injury and epithelial injury, which are hallmark events in the development of ARDS. Hence, the recruitment of LDIBs to the lung in COVID-19 likely plays an important role in the progression of ARDS observed in the most severe patients as proposed in our schematic model (FIG. 5). Increases in LDIB populations over baseline are also shown to be associated with intubation or patient mortality in our study. Conversely, a decrease in LDIB percentage frequently accompanies a positive clinical prognosis, with extubation or discharge.

Further examination into the functionality of these cells revealed a propensity for spontaneous NET formation and increased secretion of TNF-α and IL-6. Correlating these cells with clinical coagulation factors revealed that LDIBs trended with all COVID-19 patient D-dimer levels and serial analyses of patients' individual LDIB populations showed apparent associations with D-dimer. LDIB percentage also correlated with systemic IL-6 and TNFα levels as well. It is worth noting that some of these correlation analyses did not reach statistical significances. Many factors could contribute to these results. For example, our patient cohort is relatively small and many parameters such as D-dimer were not frequently measured in the clinical lab work. Nevertheless, our data suggest that LDIBs, at least in part, contribute to CAC through increased secretion of IL-6 and TNF-α particularly during LDIB crisis which results in activation of the extrinsic coagulation cascade causing thrombus formation.

In this study, we used serial patient samples taken during the length of patient hospitalization and grouped these based on the status (moderate or severe) of the patient at that time. In this way we could better capture the dynamic nature of COVID-19 in patients, and better understand how neutrophils and LDIBs change as individual patient's conditions both improve and deteriorate, and understand how severe versus moderate patients generally differ. In order to then conduct proper statistical analyses, we used linear mixed and marginal Pearson analyses to properly account for the use of these serial measurements from patients, as explained in the methods.

Recent publications in the field have called for the use of anti-inflammatory agents in the treatment of COVID-19. Numerous case reports have shown that COVID-19 patients with a history of inflammatory autoimmune diseases like rheumatoid arthritis or inflammatory bowel disease have a milder course of infection. However, in the context of the data presented here, the reduced disease severity could be a result of either drug induced neutropenia which is common in autoimmune patients or a result of decreased TNFα/IL-6 levels from monoclonal antibody treatment. There was some hesitation in the field to use immunosuppressive agents like tocilizumab, adalimumab, and etanercept due to concerns about restraining immune function during viral infection. The challenge remained in correctly identifying the patients who could benefit from immunosuppressive anti-IL-6 and anti-TNF-α therapy versus those in who it may cause delayed viral clearance resulting in worse clinical outcomes. Based on the data we present in this paper, we propose that immunosuppressive agents like tocilizumab and adalimumab, used in conjugation with anti-viral agents, could be beneficial for severe patients in or trending towards LDIB crisis to limit the deleterious effects of these cytokines on inducing coagulation. These patients can be best identified clinically by monitoring the percentage of LDIBs on routine CBCs. Obtaining a serum IL-6 level could further confirm whether a patient is trending towards an LDIB and coagulation crisis. Intervening early before patients hit this crisis could help prevent thromboembolic complications and improve all-cause mortality rates for COVID-19 patients.

### MATERIALS AND METHODS

### Study Participants and Clinical Data

The Institutional Review Board at University of Louisville approved the present study and written informed consent was obtained from either subjects or their legal authorized representatives (IRB No. 20, 0321). Inclusion criteria were all hospitalized adults (older than 18) at the University of Louisville Health who have positive COVID-19 results and were consented to this study. Exclusion criteria included age younger than 18 and refusal to participate. COVID-19 patients enrolled in this study were diagnosed with a 2019-CoV detection kit using real-time reverse transcriptase-polymerase chain reaction performed at the University of Louisville Hospital Laboratory from nasal pharyngeal swab samples obtained from patients.

The grouping of COVID-19 patients into Moderate Group vs. Severe Group is based on the initial clinical presentation at the time of enrollment. Severe Group participants were COVID-19 confirmed patients who required mechanical ventilation and this group had blood draw daily along with their standard laboratory work. Moderate Group participants were COVID-19 confirmed patients who were hospitalized without mechanical ventilation and had blood draw every two to three days along with their standard laboratory work. All COVID-19 patients were followed by the research team daily and the clinical team was blinded to findings of the research analysis to avoid potential bias.

The demographic characteristics (age, sex, height, weight, Body Mass Index (BMI), clinical data (symptoms, comorbidities, laboratory findings, treatments, complications and outcomes) and results of cardiac examinations including biomarkers, ECG and echocardiography were collected prospectively by two investigators (JH and MW). All data were independently reviewed and entered into the computer database (CW and DT). The clinical outcomes (discharge, mortality and length of stay) were monitored up to May 15, 2020. For hospital laboratory CBC tests, normal values are the following: white blood cell (4.1-10.8×10³/μL); hemoglobin (13.7-17.5gram/dL); platelet (140-370×10³/μL). For hospital laboratory inflammatory and coagulation markers, normal values are the following: D-dimer (0.19-0.74 μgFEU/ml); ferritin (7-350 ng/ml); lactate dehydrogenase (LDH) (100-242 Units/Liter).

### Plasma and PBMC Isolation

Whole blood samples were centrifuged at 1600 rpm for 10 minutes. Plasma was aspirated and aliquoted into 1 mL Eppendorf tubes and immediately stored at −80 C until future use. The remaining cell layers were diluted with an equal volume of complete RPMI1640. The blood suspension was layered over 5 mL of Ficoll-Paque (Cedarlane Labs, Burlington, ON) in a 15 mL conical tube. Samples were then centrifuged at 2,000 rpm for 30 minutes at room temperature (RT) without brake. The mononuclear cell layer was then transferred to a new 15 mL conical tubes and resuspended in 40 mL of RPM, mixed, and centrifuged at 1,500 RPM for 10 minutes at 4° C. The supernatant was removed and cells were again washed with RMP11640. The cell pellet was resuspended in 3mL of RPMI1640 and counted for sample processing.

### Whole Blood Analysis

For whole blood analysis, 150 uL of whole blood was lysed with 2 mL of ACK for 10 minutes. Cells were spun down and washed once with PBS. Cells were then stained with Viability Dye/APC-Cy7, CD45-PeCy7, CD66b-PE, and CD-16 FITC for 30 minutes at 4° C. prior to washing and analysis of a BD FACS Canto.

### Ex Vivo Neutrophil Stimulation

Whole blood (150 μL) was lysed with ACK buffer. One-million cells were seeded in a 24-well plate and cultured with. Brefeldin A solution for 20 minutes at 37° C. Cells were then stimulated with 250 ng/mL of LPS for 10 hours at 37° C. Following stimulation, cells were collected and washed with PBS prior to cell surface staining with Viability Dye-APC-Cy7, CD45-PE-Cy7, CD66b-PE, CD16-APC for 30 minutes at 4° C. Cells were washed again with PBS before fixation (Biolegend Intracellular Fixation Buffer) for 30 minutes at RT. Cells were then washed twice with permeabilization buffer (Biolegend Per Wash Buffer). Cells were incubated with TNFα-PerCP-Cy5.5 and IL-6-FITC overnight prior to washing and analysis on BD FACS Canto.

### Wright Giemsa Stain

Half-million PBMCs were stained with Viability Dye-APC-Cy7, CD45-PerCP-Cy5.5, CD66b-PE. CD16-APC for 30 minutes at 4° C. prior to washing with AutoMACs running buffer. Cells were then sorted based on CD16 expression using a BD FACS Aria 11.1. Following collection, cells were spun down at 1600 RMP for 8 minutes. Cells were resuspended in 200 μL and spun onto a microscope slide (800 rpm for 5 minutes) using a Shandon CytoSpin3 (Thermo Fisher). Slides were then air dried for 10 minutes prior to staining. For the Wright Giemsa. Stain (Shandon Wright Giemsa Stain Kit, Thermo Fisher), slides were dipped in Wright-Gietnsa Stain Solution for 1 minute and 20 seconds. After blotting off excess stain, slides were dipped in Wright Giemsa. Buffer for 1 minute and 20 seconds. Slides were blotted to remove excess buffer. Slides were then dipped into the Wright-Giemsa Rinse Solution for 10 seconds using quick dips. The back of the slides were wiped and set to dry in a vertical position for 10 minutes prior to analysis on an Aperio Scan Scope.

### CyTOF Mass Cytometry Sample Preparation

Mass cytometry antibodies (FIG. 7) were either purchased pre-conjugated (Fluidigm) or were conjugated in house using MaxPar X8 Polymer Kits or MCPS Polymer Kits (Fluidigm) according to the manufacturer's instruction. PBMCs were isolated as described above. The starting cell number was 1.0×10⁶ cells per patient. The samples were stained for viability with 5 uM cisplatin (Fluidigm) in serum free RPMI1640 for 5 minutes at RT. The cells were washed with FBS (10%) containing RPMI1640 for 5 minutes at 300×g. Cells were stained with the complete antibody panel for 30 minutes at RT. Cells were then washed and fixed in 1.6% formaldehyde for 10 minutes at RT. They were washed and then incubated overnight in 125 nM of Intercalator-Ir (Fluidigm) at 4° C.

### CyTOF Data Acquisition

Prior to acquisition, samples were washed twice with Cell Staining Buffer (Fluidigm) and kept on ice until acquisition. Cells were then resuspended at a concentration of 1 million cells/mL in Cell Acquisition Solution containing a 1/9 dilution of EQ 4 Element Beads (Fluidigm). The samples were acquired on a Helios (Fluidigm) at an event rate of <500 events/second. After acquisition, the data were normalized using bead-based normalization in the CyTOF software. The data were gated to exclude residual normalization beads, debris, dead cells and doublets, leaving DNA⁺CD45⁺Cisplatin^{low} events for subsequent clustering and high dimensional analyses.

### CyTOF Data Analysis

CyTOF data was analyzed using a combination of the Cytobank software package and the CyTOF workflow, which consists of suite of packages available in R (r-project.org). For analysis conducted within the CyTOF workflow, FlowJo Workspace files were imported and parsed using functions within flowWorkspace and CytoML. An arcsinh transformation (cofactor=5) was applied to the data using the dataPrep function within CATALYST and stored as a singlecellexperiment object. Cell population clustering and visualization was conducted using FlowSOM and ConsensusClusterPlus within the CyTOF workflow and using the viSNE application within Cytobank. Depending on the analysis, clustering was either performed using data across all donors at the first blood draw (Healthy Donors, n=5; Moderate, n=6; Severe, n=7), or using data from selected patients across multiple time points. Additionally, clustering was performed either using all live CD45+ cells or after gating on CD66b⁺ neutrophils.

### TNF-α and IL-6 Quantification

Plasma concentrations of TNFα and IL-6 were measured using enzyme-linked immunosorbent assay (ELISA) kits (BioLegend, San Diego, Calif.). The operating procedure provided by the manufacturer was followed. One-hundred μL of plasma was used for each sample. The optical density (OD) at 450 nm was measured using a Synergy™ HT microplate reader (BioTek, Winooski, Vt.). Concentrations of TNF-α and IL-6 were determined using the standard curves. A few OD readings tell outside of the range of the standard curve, in which case a line of best fit was used to extrapolate the data.

### Phagocytosis Assay

Cells were acquired from whole blood following ACK lysis. One-million cells were washed with HEPES diluted 50× in RPMI1640, and then incubated in 100 μL of this solution for 1 hour at 37° C. for activation. The pHrodo™ Green S. aureus BioParticles™ Phagocytosis Kit for Flow Cytometry was used, where 100 μL of the reconstituted particles were added to the activated whole blood, and incubated for 1 hour at 37° C. Samples were lightly mixed every 20 minutes. The reaction was stopped with 1 mL of cold PBS, and surface staining for viability, CD45, CD66b and CD16 (BioLegend, San Diego, Calif.) was performed. Samples were analyzed using a BD FACSCanto (BD Biosciences, Oxford, UK), and cells that fluoresced in the FITC channel were determined to be phagocytic,

### Statistical Analysis

First descriptive statistics such as mean and standard deviation and graphics were presented for each variable, stratified by study groups. Since we have varied number of observations for each patient, we applied linear mixed effect models along with the Wald test statistics to compare the group differences, where group was considered as fixed effects, and patients were considered random effects. To examine association between two variables, we estimated the marginal Pearson correlation coefficient and tested its significance. The marginal Pearson correlation coefficient captures the association between two variables at the population level. The analyses were carried out in the Statistical software R (https://www.r-project.org/) and Prism version 10. A statistical test was claimed significant if p<0.05.

### EXAMPLE 2

### A Specific Low-Density Neutrophil Population Correlates with Hypercoagulation and Disease Severity in Hospitalized COVID-19 Patients

Severe acute respiratory syndrome coronavirus 2 (SARS-CoV-2) is a novel viral pathogen that causes a clinical disease called coronavirus disease 2019 (COVID-19). Although most COVID-19 cases are asymptomatic or develop mild upper respiratory tract symptoms, a significant number of patients develop severe or critical disease. Patients with severe COVID-19 commonly present with viral pneumonia that may progress to life-threatening acute respiratory distress syndrome (ARDS). COVID-19 patients are also predisposed to venous and arterial thromboses that are associated with a poorer prognosis. The present study identified the emergence of a low-density inflammatory neutrophil (LDN) population expressing intermediate levels of CD16 (CD16^{Int}) in COVID-19 patients. These cells demonstrate proinflammatory gene signatures, activate platelets, spontaneously form neutrophil extracellular traps (NET), and exhibit enhanced phagocytic capacity and cytokine production. Strikingly, CD16^{Int} neutrophils are also the major immune cells within the bronchoalveolar lavage fluid, exhibiting increased CXCR3, but loss of CD44 and CD38 expression. The percent of circulating CD16^{Int} LDN is associated with D-dimer, ferritin, and systemic IL-6 and TNF-α levels and changes over time with altered disease status. Our data suggest that the CD16^{Int} LDN subset contributes to COVID-19-associated coagulopathy (CAC), systemic inflammation, and ARDS. The frequency of that LDN subset in the circulation could serve as an adjunct clinical marker to monitor disease status and progression.

In December 2019, a novel viral pathogen, severe acute respiratory syndrome coronavirus 2 (SARS-CoV-2) emerged that causes a clinical disease called coronavirus disease 2019 (COVID-19). While a majority of COVID-19 cases are asymptomatic or develop mild upper respiratory tract symptoms, studies early in the pandemic reported up to 20% of patients develop severe or critical disease. Patients with severe COVID-19 commonly develop lower respiratory tract disease due to viral pneumonia that progresses to life-threatening acute respiratory distress syndrome (ARDS) in 12% to 25% of hospitalized. patients. Fluid accumulation in the lungs that is pathognomonic for ARDS results from a combination of virally induced lung injury and the rapid influx of immune cells to fight the infection. These recruited inflammatory cells are often in a hyper-activated state associated with a phenomenon known as cytokine storm. A variety of cytokines are elevated during cytokine storm including interleukin-6 (IL-6), IL-1β, and tumor necrosis factor-α (TNFα). Levels of all three cytokines are elevated in the peripheral blood of COVID-19 patients. Persistently high levels of cytokines are associated with increased risk of vascular hyperpermeability, multi-organ failure, and death.

In addition to significant pulmonary complications, severe COVID-19 patients have a notable coagulopathy, Up to 60% of critically ill COVID-19 patients develop COVID-19-associated coagulopathy (CAC), manifested by increased D-dimer levels, no change or modestly decreased platelet count, decreased prothrombin time or partial thromboplastin time, and an increased risk of microvascular or macrovascular thrombosis. Based on the association of CAC with worse patient outcomes, high intensity thromboprophylaxis or therapeutic anticoagulation were proposed for severely ill or intubated COVID-19 patients. Although not yet peer reviewed, preliminary results from REMAP/ATTACC/ACTIV4a trials suggest a benefit of therapeutic anticoagulation in moderately ill COVID-19 patients, but not in critically ill patients. In addition, intermediate dose prophylactic anticoagulation did not lead to a significant difference in the primary outcomes in severe COVID-19 patients, compared to standard dose prophylactic anticoagulation. Thus, empiric intensification of anticoagulation in critically ill COVID-19 patients should be pursued with caution. Excessive inflammation, platelet activation, neutrophil extracellular trap (NET) formation, and endothelial dysfunction are factors postulated to induce CAC. In addition, both IL-6 and TNF-α alter platelet activation and/or the coagulation cascade which may contribute to CAC. However, the cellular and molecular pathophysiology of CAC remains to be fully elucidated.

Evidence increasingly supports a role for neutrophils in both ARDS and vascular thrombosis occurring in severe COVID-19 patients. Severe COVID-19 patients have a distinct immunological phenotype characterized by lymphopenia and neutrophilia, and an increased neutrophil to lymphocyte ratio (NLR) is associated with high D-dimer levels, enhanced vascular thrombosis, and worse clinical outcomes. Lung specimens at autopsy showed a marked infiltration of neutrophils into the lung tissue. Neutrophils are thought to be recruited to the lungs to aid in the clearance of the viral pathogens through phagocytosis, generation of reactive oxygen species (ROS), and cytotoxic granule release. However, prolonged neutrophil activation associated with delayed apoptosis and increased NET formation is linked to alveolar damage and adverse outcomes in patients with H1N1 influenza. NET formation is postulated to play a prominent role in COVID-19 intravascular coagulation.

The current study was initiated to examine the possibility that the neutrophils are significantly contributing to the coagulopathy in hospitalized COVID-19 patients. We found a marked increase in the CD66b÷ low-density neutrophils (LDN) within the peripheral blood mononuclear cell (PBMC) compartment of patients with COVID-19. Within the severe COVID-19 patient cohort, we saw the emergence of a significant population of LDN expressing intermediate levels of CD16 (CD16^{Int} LDN). A similar population of neutrophils predominated in the bronchoalveolar lavage (BAL) fluid. Transcriptomic profiling and functional analysis of CD16^{Int} LDN revealed a proinflammatory phenotype, suggesting that CD16^{Int} LDN significantly contribute to immunothrombosis and systemic inflammation in hospitalized COVID-19 patients.

### RESULTS

### Clinical Characteristics of COVID-19 Patients

In our study, a total of 53 patients who tested positive for SARS-CoV-2 by nasopharyngeal swab were screened and recruited. Additionally, 9 patients with similar comorbidities but SARS-CoV-2 negative and 6 healthy donors were recruited as controls. The study subject demographics and summary of clinical information are shown in Table 2.

TABLE 2

| | | | |
|---|---|---|---|
| | | Comorbid | |
| | Healthy | Healthy | |
| | Control | Control | COVID-19 |
| Variables | (N = 8) | (N = 9) | (N = 53) |

Sex-no (%)

| | | | |
|---|---|---|---|
| Male | 4 (66.6) | 5 (55.5) | 24 (42.3) |
| Female | 2 (33.3) | 4 (44.4) | 28 (57.7) |

Age-year

| | | | |
|---|---|---|---|
| Mean ± SD | 49.2 (15.1) | 65.9 (16.7) | 60.8 (17.75) |
| Median (IQR) | 53.5 (24.25) | 63 (24) | 63 (28.25) |
| Range | 28-68 | 41-93 | 28-95 |

BMI

| | | | |
|---|---|---|---|
| Mean ± SD | 28.75 (3.5) | 30.82 (13.1) | 31.83 (9.07) |
| Median (IQR) | 29.55 (3.13) | 29.15 (11.22) | 30.6 (14.48) |
| Range | 16.3-27 | 16.4-60.2 | 17.7-51.8 |

Ethnicity-no (%)

| | | | |
|---|---|---|---|
| AA | 0 (0) | 2 (22) | 18 (34.6) |
| White | 6 (100) | 7 (78) | 33 (63.4) |
| Hispanic | 0 (0) | 0 (0) | 1 (2) |

Time from symptoms to hospital admission, days

| | |
|---|---|
| Mean ± SD | 5.17 (5.41) |
| Median (IQR) | 4 (4.75) |
| Range | 0-14 |

Comorbidity-no (%)

| | | | |
|---|---|---|---|
| Mean ± SD | 1 (.81) | 6 (2.62) | 6.1 (3.7) |
| Median (IQR) | 1 (1.5) | 7 (4) | 4 |
| Range | 0-2 | 1-9 | 1-18 |
| hypertension | 2 (33.3) | 7 (77.7) | 39 (75) |
| Diabetes | 0 | 4 (44.4) | 27 (52) |
| Respiratory System | 0 | 6 (66.6) | 21 (40.4) |
| Cardiovascular | 0 | 4 (44.4) | 22 (42.3) |
| Disease | | | |
| Kidney Disease | 0 | 1 (11.1) | 11 (21.15) |

Treatment

| | |
|---|---|
| Hydroxychloroquine- | 8 (15.38) |
| no (%) | |
| Convalescent | 8 (15.38) |
| Plasma-no (%) | |
| Dexamethasone-no | 12 (23.1) |
| (%) | |
| Remdesivir-no % | 11 (21.15) |

For neutrophil immunophenotyping study. 10 patients were initially enrolled in the severe category, as defined by necessity of mechanical ventilation within the intensive care unit (ICU), and 21 were initially enrolled in the moderate group, as patients were admitted to the hospital but were not on mechanical ventilation. Of note, 2 of the originally enrolled moderate patients progressed to the severe category while 3 severe patients improved to be classified as moderate during the course of our study. Those patients were counted as individual patients within their original or secondary groups depending on the classification on the day blood was obtained. The study subject demographics and summary of clinical information on immunophenotyped patients are shown in Table 3.

TABLE 3

| | | | | |
|---|---|---|---|---|
| | | Comorbid | | |
| | Healthy | Healthy | COVID-19 | COVID-19 |
| Neutrophil | Control | Control | Moderate | Severe |
| phenotyping | (N = 6) | (N = 9) | (N = 24) | (N = 12) |

Sex-no (%)

| | | | | |
|---|---|---|---|---|
| Male | 4 (66.6%) | 5 (55.5%) | 13 (54%) | 6 (50%) |
| Female | 2 (33.3%) | 4 (44.4%) | 11 (46%) | 6 (50%) |

Age-year

| | | | | |
|---|---|---|---|---|
| Mean ± SD | 49.2 | 65.9 | 63.1 | 63.6 |
| | (15.1) | (16.7) | (18.55) | (15.1) |
| Range | 28-68 | 41-93 | 28-95 | 28-84 |

BMI

| | | | | |
|---|---|---|---|---|
| Mean ± SD | 28.75 | 30.82 | 31.15 | 36.4 |
| | (3.5) | (13.1) | (7.67) | (9.86) |
| Range | 16.3-27 | 16.4-60.2 | 17.7-48.8 | 21.2-49.8 |

Ethnicity-no (%)

| | | | | |
|---|---|---|---|---|
| AA | 0 (0) | 2 (22) | 9 (37.5) | 7 (58.3) |
| White | 6 (100) | 7 (78) | 15 (62.5) | 5 (41.6) |

Comorbidity-no (%)

| | | | | |
|---|---|---|---|---|
| Mean ± SD | 1 (.81) | 6 (2.62) | 4.58 (3.21) | 4.7 (3.17) |
| Range | 0-2 | 1-9 | 1-12 | 1-12 |

Peripheral blood samples were obtained daily from either a venous or arterial line for severe patients, whereas samples from moderate patients were obtained from a venous line approximately every two to three days.

### LDN are Significantly Increased in Hospitalized COVID-19 Patients and CD16^{Int} LDN are Specifically Expanded by SARS-CoV-2 Infection

Previous studies indicated a dominant neutrophilia in severe COVID-19 patients. We confirmed this finding from patient whole blood complete blood count (CBC) reports. We first partitioned all serial blood draws from each patient based on whether they were classified as moderate or severe on the day blood was obtained, and then averaged data by classification for each patient. These data demonstrated that there was an approximately 10% increase in neutrophil percentage in the peripheral blood of patients at severe time points compared to moderate time points, and a 30% increase in neutrophil percentage over what was observed in healthy donors (FIG. 20). Conversely, the overall lymphocyte percentage in patients at severe time points was decreased as compared to the moderate time points and healthy donors. Interestingly, comorbidity control patients also showed a decreased lymphocyte percent (FIG. 20A). viSNE analysis of the overall CD3⁺ T cells and CD4⁺ and CD8⁺ T cell subsets showed a decreasing population size in patients with moderate and severe COVID-19 as well as in comorbidity control patients as compared to healthy donors (FIG. 20B). Our data showing an increased NLR within our severe cohort agrees with previously published reports.

Analysis of the neutrophil pool revealed three distinct subpopulations within whole blood samples that clustered by CD16^{Low}, CD16^{Int}, and CD16^{High} expression. Severe COVID-19 patients showed a marked increase in the CD16^{Int} subset, which was significantly lower in the moderate cohort and comorbidity controls, and virtually absent in the healthy donors (FIG. 13A). CD16^{Int} neutrophils classically are reported to be low-density neutrophils (LDN) or immature neutrophils. Clinically, immature neutrophils are called band cells and are associated with a left shift on a CBC. These neutrophils are often mononucleated and smaller than typical neutrophils. Therefore, the presence of these cells in peripheral blood mononuclear cells (PBMC) isolated by Ficoll gradient separation was examined. Cell lineage cluster analysis from total PBMC population assessed by CyTOF mass cytometry indeed demonstrated that CD66b⁺ neutrophils (large circles, FIG. 13B) were the most prominent population in COVID-19 patients. Minimal LDN were seen in PBMC preparations of healthy donors (FIG. 13B). We also identified a specific population within the neutrophil cluster which showed significant expansion only in COVID-19 patients (small circles, FIG. 13B).

We further examined CD16 expression on neutrophils from the PBMC preparation. Similar to the whole blood samples. LDN in the PBMC also showed three populations based on CD16 expression (FIG. 13C). Despite increased overall neutrophils in comorbidity controls, CD16^{Int} neutrophils were only increased in moderate and severe COVID-19 patients (FIG. 13C), suggesting that SARS-CoV-2 infection specifically drives expansion of this subset of neutrophils. Cluster analysis of isolated PBMCs from a single blood draw from each subject indicated a predominate neutrophil population within the CD45⁺ compartment in the severe and moderate COVID-19 cohorts, as compared to comorbidity control patients (large circles, FIG. 13D, left panels). Additionally, there was a subset of the neutrophil population expressing intermediate CD16 levels in COVID-19 patients, which was almost absent in comorbidity control patients (small circles, FIG. 13D, right panels).

### Phenotypic Characterization of CD16^{Int} LDN

Maturation of neutrophils from hematopoietic stem cells is identified by stages with distinct morphological characteristics. We performed Wright-Giemsa staining to determine if the three CD16 populations of neutrophils were actually neutrophils in the later three stages of development: myelocyte, metamyleocyte (band cell), and granulocyte (mature neutrophil). FIG. 14A clearly shows that the CD16^{Low} cells were basophilic myelocytes with an ovoid nucleus, the CD16^{Int} cells contained a large number of band cells with the characteristic band shaped nucleus, and the CD16^{High} cells were segmented, mature neutrophils. It is worth noting, however, that the mature CD16^{High} neutrophils were typically bi-lobed rather than hyper-segmented and closely resembled pseudo-Pelger-Huet cells described in other severe infections like influenza A, tuberculosis, and human immunodeficiency virus (HIV).

Next, we explored differential surface marker expression on the different CD16⁺ neutrophil subsets from COVID-19 patients. A cluster analysis of the overall CD66b⁺ neutrophil population showed an increased prevalence of cluster 13 in the COVID-19 patient cohorts, as compared to comorbidity controls and healthy donors (FIG. 14B, circles). The single cell marker expression profiles (FIG. 21) revealed that cluster 13 showed decreased expression of CD44, CD16, and CD11b. Tracking the neutrophil clusters in serial blood draws over 5 days from different types of patients revealed the dynamic nature of neutrophil pools in COVID-19 infection (FIGS. 22A, 22B). In the severe patient, the light blue population (cluster 4, upper black circle) increased over time while all the other clusters remained similar. For the moderate patient, the majority of clusters remained stable over time. The patient who was initially enrolled in the severe cohort, but improved to moderate by day 5, had a profound decrease in cluster 5 (lower red circle) over time. Conversely, in the patient that transitioned from moderate to severe, the light blue (cluster 4) and purple clusters (cluster 5) increased over time, which was consistent with the change in disease severity.

As the profile of neutrophil clusters associates with disease status, we next determined specific surface marker phenotypes fix the different CD16 neutrophil clusters using mass cytometry. As compared to CD16^{High} LDN, CD16^{Int} LDN expressed an intermediate level of CD11b and an elevated level of CD38, CD40, CXCR5, and CD69, suggesting a more activated phenotype (FIG. 14C). In addition, CD 161^{Int} LDN showed markedly downregulated CD44 expression (FIG. 14C). Cluster analysis revealed that the CD161^{Int} LDN cluster (blue circle) indeed showed decreased CD11b and CD44 expression as compared to the CD16^{High} LDN cluster (large circle, FIG. 14D). CD44 is an adhesion receptor for extracellular matrix that has been associated with neutrophilic lung inflammation in bacterial pneumonia. Consistent with the observation that decreased surface expression of CD44 resulted in increased accumulation of neutrophils in the lungs of E. coli infected mice, neutrophils from severe patients had the lowest expression of CD44 (FIG. 14E).

### CD16^{Int} LDN Exhibit Proinflammatory Gene Signatures with Increased Phagocytic Capacity and Spontaneous NET Formation

To define gene signatures of LDN subsets in COVID-19 patients, we sorted both CD16^{High} and CD16^{Int} LDN from three severe COVID-19 patients. Normal density neutrophils (NDN) were obtained from healthy donors. RNA was extracted from each neutrophil population and RNA sequencing was performed. Principal component analysis (PCA) showed striking differential aggregations among the three populations (FIG. 23A). We focused our comparison on CD16^{high} and CD16^{Int} LDN from COVID-19 patients. A total of 6387 differentially expressed genes (DEG) was observed comparing CD16^{Int} to CD16^{High} LDN (3116 upregulated DEGs and 3271 downregulated DEGs, FIG. 15A). GO biological pathway (BP) analysis showed that the neutrophil activation, neutrophil activation involved in immune response, neutrophil degranulation, and neutrophil-mediated immunity were ranked as the Top 4 enriched pathways in these DEGs (FIG. 15B). DEGs related to neutrophil activation and neutrophil activation involved immune responses were shown between Ca16^{High} and CD16^{Int} LDN (FIG. 23B). Gene set enrichment analysis (GSEA) indicated that genes related to chronic inflammatory response, positive regulation of inflammatory response, positive regulation of myeloid leukocyte mediated immunity, superoxide generation, positive regulation of leukocyte degranulation, respiratory burst, regulation of neutrophil chemotaxis, and phagocytosis recognition were significantly enriched in CD16^{Int} LDN compared to CD16^{High} neutrophils (FIG. 23C). We specifically compared DEGs related to neutrophil degranulation, NET formation, phagocytosis, signaling, and neutrophil trafficking and function (FIG. 15C). Genes related to neutrophil degranulation, NET formation, and neutrophil phagocytosis were uniformly upregulated in CD16^{Int} LDN. On the other hand, DEGs related to neutrophil trafficking did not show a consistent pattern. CD44 was downregulated, consistent with our flow cytometry data. VEGFA and ARG1were upregulated, while gasdermin. D (GSDMD) was downregulated in the CD16^{Int} LDN.

As the transcriptomic analysis revealed increased expression of phagocytic genes, we next investigated the phagocytic functionality of the neutrophils from COVID-19 patients. FIG. 15D shows that CD16^{Int} LDN had a significantly greater uptake of pHrodo green S. aureus bioparticles than CD16^{High} neutrophils, suggesting an activated phenotype. Another neutrophil anti-microbial mechanism is the formation of NETs, the extravasation of DNA and protein to form a web like structure that can trap and kill extracellular pathogens. NET formation also contributes to increased platelet aggregation and coagulation. Consistent with upregulation of NET forming genes in this subset (FIG. 15C), we observed that CD16^{Int} LDN spontaneously formed NETs (FIG. 15E). Taken together, our gene expression, protein expression, and functional data indicate that CD16^{Int} LDN exhibit a proinflammatory phenotype, including enhanced phagocytosis, NET formation, and granule mobilization and altered expression of surface molecules that may regulate their migration into the lung.

### CD16^{Int} LDN Interact with Platelets for Activation Leading to Hypercoagulable State in Severe COVID-19 Patients

A thrombogenic coagulopathy is associated with COVID-19 and the majority of severe COVID-19 patients present with elevated D-dimer levels. A recent study documented the interaction of NET-forming neutrophils with platelets in pulmonary microthrombi in autopsy specimens and found higher levels of circulating neutrophil-platelet aggregates in patients with

COVID-19. Our GSEA analysis showed that genes related to platelet morphogenesis, platelet aggregation, platelet degranulation, and platelet activation were enriched in CD16^{Int} LDN (FIG. 16A). To determine whether LDN directly interact with platelets, we quantified circulating neutrophil-platelet aggregates in the whole blood samples from additionally recruited COVID-19 patients (Table 4).

TABLE 4

| | |
|---|---|
| Platelet | COVID-19 |
| study | Patients |

Sex-(Total, n = 13)

| | |
|---|---|
| Male | 7 (53.8%) |
| Female | 6 (46.2%) |

Age-year

| | |
|---|---|
| Mean ± SD | 52.15 ± (20.21) |
| Range | 21-84 |

BMI

| | |
|---|---|
| Mean ± SD | 32.4 ± (8.8) |
| Range | 21.1-45.9 |

Ethnicity

| | |
|---|---|
| White | 11 (84.6%) |
| African | 2 (15.4%) |
| American | |

Comorbidities

| | |
|---|---|
| Mean ± SD | 6.23 ± (4.16) |
| Range | 1-18 |

Neutrophil-platelet aggregates were present in both CD16^{high} and CD16^{Int} neutrophil populations (FIG. 16B). To determine the activation status of platelets in those aggregates, expression of CD62P and CD40 by platelets within aggregates was determined by flow cytometry. Both CD62P (FIG. 16C) and CD40 (FIG. 160) expression were significantly higher in CD16^{Int} neutrophil-platelet aggregates, compared to CD16^{High} neutrophil-platelet aggregates, indicating that aggregation with CD16^{Int} neutrophils is associated with significantly greater platelet activation.

The CD40-CD40L, pathway drives platelet activation and thrombosis. Inhibition of the neutrophil-platelet CD40/CD40L axis with anti-CD40 Ab is reported to significantly reduce pulmonary edema and platelet activation and reduce neutrophil recruitment to the lungs in a mouse model of transfusion related acute lung injury (TRALI). We found severe COVID-19 patients had significantly more CD40-1CD16^{Int} LDN than moderate patients as assessed by flow cytometry (FIG. 16E). Moreover, increased CD4 expression by CD16^{Int} LDN significantly correlated with increased D-dimer levels in severe COVID-19 patients (FIG. 16F). These results suggest that CD16^{Int} LDN may participate in COVID-19 coagulopathy through direct activation and aggregation of platelets.

### CD16^{Int} Neutrophils Predominate in Bronchoalveolar Lavage (BAL) Fluid

Neutrophils were observed in alveoli and interstium of lungs of autopsied COVID-19 patients and were prevalent in BAIL fluid from severe COVID-19 patients. To determine if the emergent LDN population we identified in the peripheral blood is associated with increased LDNs in the lungs, we collected BAL, fluid from severe COVID-19 patients (Table 5).

TABLE 5

| | |
|---|---|
| | COVID-19 |
| BAL study | Patients |

Sex-(Total, n = 6)

| | |
|---|---|
| Male | 2 (33%) |
| Female | 4 (66%) |

Age-year

| | |
|---|---|
| Mean ± SD | 63.67 ± (14.00) |
| Range | 40-83 |

BMI

| | |
|---|---|
| Mean ± SD | 35.36 ± (8.65) |
| Range | 22.2-45.8 |

Ethnicity

| | |
|---|---|
| White | 4 (66%) |
| African | 2 (33%) |
| American | |

Comorbidities

| | |
|---|---|
| Mean ± SD | 5 ± (3) |
| Range | 0-9 |

Neutrophils constituted the major immune cell population within the BAL fluid. Strikingly, CD16^{Int} neutrophils accounted for more than 60% of the total neutrophil population in BAL fluid (FIG. 17A). In addition, almost all CD16^{Int} neutrophils in the BAL fluid expressed significantly lower levels of CD44 than peripheral blood CD16^{Int} 41 neutrophils from the same patient (FIG. 17B). Comparison of CD16^{Int} neutrophils from peripheral blood and BAL fluid from the same patient identified two additional markers that were differentially expressed. CD16^{Int} neutrophils in BAL fluid expressed significantly greater levels of the chemokine receptor CXCR3, while CD38 was markedly downregulated (FIG. 17C). We also found that CXCR3 expression was higher in CD16^{Int} neutrophils compared to that in CD16^{High} population (FIG. 24A). In contrast, CD44 expression levels were lower in CD16^{Int} neutrophils compared to these in CD16^{High} subset in BAL samples (FIG. 24A). The expression levels of IL-7Ra and degranulation marker LAMP-1 were also marginally increased in CD16^{Int} neutrophils from BAL fluid (FIG. 17C). A previous study showed that CXCR3 is expressed on lung-recruited neutrophils during influenza pneumonia, CD38 is an ADP-ribosyl cyclase that controls neutrophil chemotaxis to bacterial chemoattractants. Loss of CD38 on CD16^{Int} could contribute to a mechanism where these neutrophils may accumulate in the lungs due to chemokine signaling and without CD38 expression then lack the ability to exit the lungs, leading to neutrophil accumulation.

To evaluate possible stimuli for CD16^{Int} neutrophil trafficking from periphery to the lung, we assayed chemokines/cytokines in the BAL fluid. High levels of a number of chemokines and cytokines capable of recruiting or activating neutrophils were present in the BAL fluid, including G-CSF. IL-1RA, IP-10, MCP-1 and IL-8 (FIG. 17D). Proinflammatory cytokines IL-6 and TNF-α were also present at high concentrations. Consistent with previous studies showing deficient expression of interferon-stimulated genes suggesting defective anti-viral immune responses in severe COVID-19. type I IFNs including IFN-α2a and IFN-β were not detectable. Levels of IP-10, G-CSF, IL-8 and VEGFA were significantly increased in the BAL fluid compared to the corresponding plasma samples (FIG. 17E). IP-10 (CXCL10) is a chemokine ligand for CXCR3, which as noted above was highly expressed on CD16^{Int} neutrophils from BAL fluid. Collectively, these preliminary data suggest that the CXCL10-CXCR3 axis may participate in CD16^{Int} neutrophil recruitment into the lungs of COVID-19 patients.

### Frequency of CD16^{Int} LDN is Correlated with Plasma Levels of IL-10, IL-1R, MCP-1, and MIP-b 1α

To screen for mediators responsible for expanding the CD16^{Int} neutrophils population, we measured 20 cytokineslchemokines in COVID-19 patient plasma samples (Table 6).

TABLE 6

| | |
|---|---|
| | COVID-19 |
| Plasma | Patients |

Sex-(Total, n = 36)

| | |
|---|---|
| Male | 14 (39%) |
| Female | 22 (61%) |

Age-year

| | |
|---|---|
| Mean ± SD | 63.3 ± (17.09) |
| Range | 28-95 |

BMI

| | |
|---|---|
| Mean ± SD | 31.51 ± (8.9) |
| Range | 17.7-49.8 |

Ethnicity

| | |
|---|---|
| White | 21 (58.3%) |
| African | 14 (38.9%) |
| American | |
| Hispanic | 1 (2.7%) |

Comorbidities

| | |
|---|---|
| Mean ± SD | 5.5 ± (6.04) |
| Range | 1-18 |

As shown in FIG. 24B, plasma levels of IL-10, IL-1RA, MCP-1 and MIP-1α positively correlated with the percentage of CD16^{Int} neutrophils, while correlating negatively with the percentage of CD16^{High} neutrophils. No correlations were noted in CD neutrophils population (data not shown). These four cytokines/chemokines are likely to he involved in neutrophil trafficking and migration. Therefore, it remains to be determined whether these factors contribute to emergence of CD16^{Int} neutrophils in severe COVID-19 patients.

### Contribution of CD16^{Int} LDN to Systemic Cytokine Production in COVID-19 Patients

Severe COVID-19 patients have elevated levels of pro-inflammatory cytokines resulting in cytokine storm. Two cytokines found to be consistently elevated among the most severe COVID-19 patients are TNF-α and IL-6. In addition to their effect on innate immunity, both IL-6 and TNF-α activate the extrinsic coagulation cascade by inducing endothelial cell expression of tissue factor. As these activities may contribute to COVID-19 coagulopathy, we determined if CD16^{Int} LDN and/or overall neutrophils contributed to the generation of these cytokines and whether they correlated with clinical markers of coagulation and systemic inflammation. Although plasma levels of TNF-α remained low in COVID-19 patients, TNF-α levels were significantly higher in the severe COVID-19 group, compared to healthy donors. IL-6 levels in severe COVID-19 patients were significantly increased above those in moderate COVID-19 patients, comorbidity control patients, and healthy donors (FIG. 18A). Plasma levels of TNF-α and IL-6 did not significantly correlate with total neutrophil percentage (FIG. 18B). The percentage of CD16^{Int} LDN, however, showed a significant positive correlation with TNF-α and IL-6 levels across all COVID-19 patients (FIG. 18C).

Next, we examined whether neutrophils directly contribute to these systemic cytokine pools. CD16^{Int} neutrophils in the severe patients released higher amounts of TNF-α, and IL-6, compared to moderate or comorbidity control patients (FIG. 18D). Additionally, neutrophils from severe COVID-19 patients accounted for an increased proportion of cytokine-producing cells, compared to comorbidity control patients (FIG. 18E). TNF-α levels demonstrated a significant correlation with platelet counts and LDH levels, but no correlation with D-dimer and ferritin (FIG. 25A). In contrast, IL-6 levels were positively correlated with the levels of D-dimer, ferritin and LDH and negatively correlated with platelet count (FIG. 25B). Overall, these data suggest that neutrophils, particularly the CD16^{Int} LDN subset, are important contributors to the elevated cytokine levels seen in COVID-19 patients.

### Clinical Significance of CD16^{Int} Neutrophils in COVID-19 Patients

Two clinical markers used to monitor coagulation state are D-dimer and platelet count, where increased D-dimer levels and decreased platelet counts are associated with enhanced coagulation. Our severe COVID-19 cohort showed elevated D-dimer levels, compared to those with moderate disease (FIG. 19A). Platelet counts were similar between two groups. Two clinically relevant markers used to monitor systemic inflammation are ferritin and lactate dehydrogenase (LDH). Ferritin levels were elevated above the normal range in our COVID-19 patients, however, there was no difference between patients with moderate and severe disease. LDH levels were similar in the severe cohort versus moderate group (FIG. 19A).

To determine if total neutrophil percentage can identify patients with a high risk of thromboembolism, the neutrophil percentage was correlated with D-dimer, ferritin, platelet count, and LDH levels. There was no significant correlation between neutrophil percent and any of these markers (FIG. 19B). In contrast, the CD16^{Int} neutrophil percent significantly correlated with D-dimer and ferritin levels, while there was no correlation with platelet count or LDH level (FIG. 19C). Longitudinal analyses of individual patient's CD16^{Int} neutrophil populations with D-dimer demonstrated a significant relationship and a pronounced phenotype. FIG. 26A shows a representative severe patient in whom rising D-dimer levels correlated with an increasing CD16^{Int} neutrophil population within their peripheral blood until their death (FIG. 26A). In contrast, both D-dimer and CD16^{Int} LDN percentage in one patient from the moderate group were only marginally elevated throughout the hospital stay until discharge (FIG. 26B). These findings suggest that the CD16^{Int} neutrophil percentage rather than overall neutrophil percentage correlates with coagulation status and clinical outcome in COVID-19 patients.

Tracking the CD16^{Int} neutrophil population over the course of each patient's hospital stay revealed an association between clinical outcomes and the percentage of CD16^{Int} neutrophils (FIG. 19D-19F). The longitudinal blood samples were collected from 25 patients and the frequency of CD161^{Int} LDN was monitored over time. In patients who died, the percentage of CD16^{Int} LDN increased over time and reached the highest level on the last sample obtained before death (FIG. 191)). For patients recovering from COVID-19, two scenarios are observed. One group of patients showed an initial high percentage of CD10 LDN that gradually decreased to basal levels prior to discharge (FIG. 19E). A second group of patients showed low percentages of CD16^{Int} LDNs for the duration of the hospitalization until discharge (FIG. 19F). Collectively, these findings suggest that an emergence of CD16^{Int} LDNs is common in COVID-19 patients, and that changes in the percent of CD16^{Int} LDNs predicts both improvement and decline in clinical status.

### Discussion

The primary finding of our study is the emergence of a subpopulation of LDN in COVID-19 patients that associates with disease severity and changes over time in parallel with changing coagulation and clinical status. Although our severe COVID-19 patients showed an increased neutrophil percentage and increased NLR, neither of these measurements were associated with coagulation status. We describe the emergence of a unique LDN subpopulation in COVID-19 patients. Previous studies have shown that LDN are expanded in severe infection and autoimmune disorders such as lupus. Indeed, comorbidity COVID-19^{neg} control patients have significantly increased LDN within the PBMC population. However, LDN are a heterogenous population that can be further classified as CD16^{High}, CD16^{Int}, and CD16^{Low}. Our study shows that CD16^{Int} LDN are only increased in COVID-19 patients, suggesting that SARS-CoV-2 infection specifically drives expansion of this subset. In addition, severe patients have a greater percentage of CD161^{Int} LDN than moderate patients, indicating that CD16^{Int} LDN are correlated with disease severity. Our data expand on the findings of two recent studies showing emergence of dysfunctional LDNs in severely ill COVID-19 patients.

LDN are classically considered to be immature neutrophils, and our CD16^{Int} LDN population show a band shaped nucleus, resembling immature neutrophil morphology. Although previous studies suggested the emerging neutrophils are immature with phenotypic signs of immunosuppression and dysfunction, our RNAseq data reveal that the CD16^{Int} LDN have a potent proinflammatory gene signature and demonstrate increased neutrophil degranulation, NET formation, and phagocytosis. NET formation has been reported in severe COVID-19 pulmonary autopsies. Serum levels of cell-free DNA, DNA-MPO complexes and citrullinated histone 1-13 are increased in COVID-19 patients, further supporting the notion that NETs play a critical role in lung immunopathogenesis in severe COVID-19 patients. In addition to expression of NET-related genes, we observe that CD16^{Int} LDN spontaneously form large numbers of NETs. Collectively, our findings indicate that CD16^{Int} LDN are morphologically immature but functionally competent with a hyper-activated phenotype.

Evidence suggests that neutrophils aggregate with platelets in COVED-19 leading to microvascular thrombosis and subsequent lung damage. Our data show that neutrophil-platelet aggregates contain both CD16^{High} and CD16^{Int} neutrophils, however, a higher percent of platelets with activation markers are present in the CD16^{Int} neutrophil aggregates. This is consistent with RNAseq data showing genes related to platelet activation and degranulation are enriched in CD16^{Int} LDN. Additionally, CD40 expression is higher in these aggregates, and the frequency of CD40⁺-CD16^{Int} LDN highly correlates with D-dimer levels in COVID-19 patients. Although it is possible that platelet activation could activate neutrophils, however, a recent study suggests that the activation status of neturophils is more important than platelet activation in COVID-19-related thrombosis. Overall, our results suggest that CD16^{Int} neutrophils may he capable of promoting coagulation and thrombosis and could play a prominent role in CAC, though future studies are needed to show a direct connection between CD16^{Int} neutrophils and the formation of platelet aggregates.

Neutrophil infiltration of the lung is accompanied by lung edema, endothelial injury, and epithelial injury, which are hallmark events in the development of ARDS. Our finding that neutrophils are the major immune cells in the BAL fluid from severe COVID-19 patients is consistent with previous reports. In the six patients analyzed, we show that the CD16^{Int} neutrophil subpopulation consistently constitutes more than 60% of neutrophils in the BAL fluid. Those CD16^{Int} BAL fluid neutrophils express CXCR3, but lose CD44 and CD38 expression, compared to CD16^{Int} neutrophils in the blood. In addition, CD16^{Int} BAL fluid neutrophils express higher levels of CXCR3 than CD16^{High} population. The elevated potent neutrophil chemoattractant, including the CXCR3 ligand IP-10 (CXCL10), in the BAL fluid may preferentially recruit CXCR3+CD16^{Int} neutrophils into alveoli and BAL fluid. The mechanism by which CD16^{Int} neutrophils recruited to the lungs lose CD44 and CD38 expression is unknown, however, neutrophils undergoing transmigration from the vasculature undergo a number of phenotypic changes, including release of proteolytic enzymes. The downregulation of CD44 may enhance trafficking of these cells into the lung, as previous studies report that CD44-deficient mice show markedly increased migration of neutrophils into the lungs after induction of bacterial pneumonia or hypoxia-induced injury. Strikingly, CD16^{Int} neutrophils from BAL fluid completely lose CD38 expression. CD38 was reported to play a role in neutrophil chemotaxis to bacterial formylated peptide chemoattractant. Our results suggest the hypothesis that reduced CD38 expression may inhibit CD16^{Int} neutrophil chemotaxis, thereby limiting their emigration from the lung. BAL fluid also demonstrates significant levels of TNF-α and IL-6. Our data show that CD16^{Int} neutrophils are capable of producing increased levels of these cytokines compared to comorbidity controls. Hence, the recruitment of CD16^{Int} neutrophils to the lung in COVID-19 may also play an important role in cytokine production leading to the development of ARDS observed in the most severely ill COVID-19 patients.

To address the question of which mediators are responsible for expanding the CD16^{Int} neutrophils population, we measured the levels of cytokines/chernokines in COVID-19 patient plasma samples. The plasma levels of IL-10,11,-1RA, MCP-1 and MIP-1α positively correlated with the percentage of CD16^{Int} neutrophils while negatively correlated with the percentage of CD16^{High} neutrophils. Interestingly, a recent study reported that IL-10 and IL-1RA levels are associated with disease severity in COVID-19 patients using longitudinal blood samples. In addition, a previous report also showed that ICU patients had higher plasma levels of MCP-1 and MIP-1α. Collectively, these correlation studies further support our conclusion that CD16^{Int} neutrophils play a critical role in disease development and progression. Although the levels of these four cytokines/chemokines significantly correlate with percentages of CD16^{Int} neutrophils, it is currently unknown whether these cytokines actually stimulate expansion of CD16^{Int} neutrophils in severe COVID-19 patients.

Recent publications promoted the use of anti-inflammatory agents in the treatment of COVID-19, Numerous case reports suggest that COVID-19 patients with a history of inflammatory autoimmune diseases like rheumatoid arthritis or inflammatory bowel disease have a milder course of infection. In the context of the data presented here, the reduced disease severity in autoimmune diseases could he due to drug induced neutropenia or to decreased TNF-α/IL-6 levels from antibody treatment. Hesitation to use cytokine blocking antibodies like tocilizumab, adalitnurnab, and etanercept, exists due to concerns that restraining immune function will promote the viral infection. The results with dexamethasone treatment, however, have shifted opinion toward acceptance of immune modulation and suppression as successful treatment. However, the challenge to correctly identify patients who could benefit from immunosuppressive regimens like dexamethasone or anti-IL-6 therapy remains. Based on the data we present here, we propose that CD16^{Int} LDN levels could serve as a predictor of risk for progressive ARDS and CAC, thus, identifying patients in whom implementation of anti-inflammatory therapy may be beneficial.

### METHODS

### Study Participants and Clinical Data

The Institutional Review Board at University of Louisville approved the present study and written informed consent was obtained from either subjects or their legal authorized representatives (IRB No. 20, 0321). Inclusion criteria were all hospitalized adults (older than 18) who have positive COVID-19 results and were consented to this study. Exclusion criteria included age younger than 18 and refusal to participate. COVID-19 patients enrolled in this study were diagnosed with a 2019-CoV detection kit using real-time reverse transcriptase-polymerase chain reaction performed at the University of Louisville Hospital Laboratory from nasal pharyngeal swab samples obtained from patients. The grouping of COVID-19 patients into Moderate Group vs. Severe Group is based on the initial clinical presentation at the time of enrollment. Severe Group participants were COVID-19 confirmed patients who required mechanical ventilation and this group had blood drawn daily along with their standard laboratory work. Moderate Group participants were COVID-19 confirmed patients who were hospitalized without mechanical ventilation and had blood drawn every two to three days along with their standard laboratory work. All COVID-19 patients were followed by the research team daily and the clinical team was blinded to findings of the research analysis to avoid potential bias.

The demographic characteristics (age, sex, height. weight, Body Mass index (BMI) and clinical data (symptoms, comorbidities, laboratory findings, treatments, complications and outcomes) were collected prospectively. All data were independently reviewed and entered into the computer database. For hospital laboratory CBC tests, normal values are the following: white blood cell (4.1-10.8×10³/μL); hemoglobin (13.7-17.5 g/dL); platelet (140-370×10³/μL). For hospital laboratory inflammatory and coagulation markers, normal values are the following: D-dimer (0.19-0.74 μg/ml FEU); ferritin (7-350 ng/ml); LDH (100-242 Units/Liter).

### Plasma and PBMC Isolation

Whole blood samples were centrifuged at 1600 rpm for 10 min. Plasma was aspirated and aliquoted into I mL Eppendorf tubes and immediately stored at −80° C. until future use. The remaining cell layers were diluted with an equal volume of complete RPMI1640. The blood suspension was layered over 5 mL of Ficoll-Paque (Cedarlane Labs, Burlington, ON) in a 15 mL conical tube. Samples were then centrifuged at 2,000 rpm for 30 min at room temperature (RI) without brake. The mononuclear cell layer was then transferred to a new 15 mL conical tubes and washed with complete RPMI 1640. The cell pellet was resuspended in 3 mL of RPMI1640 and counted for sample processing.

### Whole Blood Analysis

For whole blood analysis, 150 uL of whole blood was lysed with 2 mL of ACK buffer for 10 min. Cells were spun down and washed once with PBS. Cells were then stained with Viability Dye/APC-Cy7, CD45-PeCy7, CD66b-PE, and CD-16 APC (Biolegend, San Diego, Calif.) for 30 min at 4° C. prior to washing and analysis of a BD FACSCanto (BD Biosciences).

### CyTOF Mass Cytometry Sample Preparation

Mass cytometry antibodies (FIG. 7) were either purchased pre-conjugated (Fluidigm) or were conjugated in house using MaxPar XS Polymer Kits or MCPS Polymer Kits (Fluidigm) according to the manufacturer's instruction. PBMCs were isolated as described above. The samples were stained for viability with 5 uM cisplatin (Fluidigm) in serum free RPMI1640 for 5 min at RT. The cells were washed with complete RPMI1640 for 5 min and stained with the complete antibody panel for 30 min at RT. Cells were then washed and fixed in 1.6% formaldehyde for 10 min at RT, and then incubated overnight in 125 nM of Intercalator-Ir (Fluidigm) at 4° C.

### CyTOF Data Acquisition

Prior to acquisition, samples were washed twice with Cell Staining Buffer (Fluidigm) and kept on ice until acquisition. Cells were then resuspended at a concentration of 1 million cells/mL in Cell Acquisition Solution containing a 1/9 dilution of EQ 4 Element Beads (Fluidigm). The samples were acquired on a Helios (Fluidigm) at an event rate of <500 events/second. After acquisition, the data were normalized using bead-based normalization in the CyTOF software. The data were gated to exclude residual normalization beads, debris, dead cells and doublets, leaving DNA⁺CD45⁺Cisplatin^{low} events for subsequent clustering and high dimensional analyses.

### CyTOF Data Analysis

CyTOF data was analyzed using a combination of the Cytobank software package and the CyTOF workflow, which consists of suite of packages available in R (r-project.org). For analysis conducted within the CyTOF workflow, FlowJo Workspace files were imported and parsed using functions within flowWorkspace and CytoML. arcsinh transformation (cofactor=5) was applied to the data using the dataPrep function within CATALYST and stored as a singlecellexperiment object. Cell population clustering and visualization was conducted using FlowSOM and ConsensusClusterPlus within the CyTOF workflow and using the viSNE application within Cytobank. Clustering was performed using data across all donors and time points. Additionally, clustering was performed either using all live CD45⁺ cells or after gating on CD66b⁺ neutrophils.

### Wright Giemsa Stain

Half million PBMC were stained with Viability Dye-APC-Cy7, CD45-PerCP-Cy5,5, CD66b-PE, CD16-APC for 30 min at 4° C. Cells were then sorted based on CD16 expression using a BD FACS Aria III. Following collection, cells were spun down at 1600 RMP for 8 min. Cells were resuspended in 200 uL and spun onto a microscope slide using a Shandon CytoSpin3 (Thermo Fisher). Slides were then air dried for 10 min prior to staining. For the Wright Giemsa Stain (Shandon Wright Giemsa Stain Kit. Thermo Fisher), slides were dipped in Wright-Giemsa Stain Solution for 1 min and 20 seconds. After blotting off excess stain, slides were dipped in Wright Giemsa Buffer for 1 min and 20 seconds. Slides were blotted to remove excess buffer. Slides were then dipped into the Wright-Giemsa Rinse Solution for 10 seconds using quick dips. The back of the slides were wiped and set to dry in a vertical position for 10 min prior to analysis on an Aperio Scan Scope.

### RNA Extraction and Sequencing

PBMCs from severe COVID-19 patients were washed and stained with Viability Dye-APC-Cy7, CD45-PerCP, CD66b-PE, CD16-APC for 30 min. at 4° C. CD16^{High} and CD16^{Int} CD66b⁺ neutrophils were sorted by a BD FACSAria III. Cells were then lysed in TRIzol and RNAs were extracted with a QIAGEN RNeasy Kit (RIAGEN). Libraries were prepared using the Universal Plus mRNA-Seq with NuQuant (NuGen). Sequencing was performed on the University of Louisville Brown Cancer Center Genomics Core Illumina NextSeq 500 using the NextSeq 500/550 75 cycle High Output Kit v2.5. The RNAseq data have been deposited into NCBI GEO with the accession number (GSE154311).

### Phagocytosis Assay

Cells were acquired from whole blood following ACK lysis. The pHrodo™ Green S. aureus BioParticles™ Phagocytosis Kit (Thermo-Fisher) was used, where 100 μL of the reconstituted particles were added to the cell suspension and incubated for 1 hour at 37° C. Samples were lightly mixed every 20 min. The reaction was stopped with 1 mL of cold PBS. Cells were then stained for viability, CD45, CD66b and CD16 (BioLegend). Samples were acquired by FACSCanto.

### NET Assay

NET formation was tested using confocal microscopy. Sorted CD16^{Int} (0.5×10⁶cell/well) were resuspended in NETs media (colorless RPMI+0.5% BSA+10 mM HEPES) and seeded onto sterile acid-washed coverslip coated with (1 mg/ml) poly-L-lysine, cells were incubated for 60 min in CO₂ incubator. Following incubation time cells were fixed with 2% PFA for 30 min, washed twice with, and blocked in 1% BSA in PBS for 1 hour at room temperature. NETs were determined by extracellular colocalization of antihuman lactoferrin antibody (1:500 dilution, MP Biomedicals) 4,6-diamidino-2-phenylindole (DAPI, 600 nM for 10 min) nuclear stain. The secondary antibody utilized was Alexa Fluor 647 (1:1,000; Life Technologies). Confocal images and Z-stacks (1 μm thickness for each slice) were obtained by the Fluoview FV1000 confocal microscope with the 63-x oil objective. Confocal Z-stack images were used to quantify co-localization of extracellular DNA and lactoferrin using IMARIS v9.6 software (Oxford Instruments, Zurich).

### Neutrophil-platelet Aggregates

Whole blood samples from COVID-19 patients were diluted with Tyrodes/HEPES buffer at 1:5. Cells were stained with anti-human CD66b, CD16, CD40, platelet marker anti-human CD41, and platelet activation marker anti-human CD62P for 10 min at RT in the dark. Cells were fixed with 1% paraformaldehyde for 10 min and then acquired by FACSCanto.

### BAL Fluid Collection

Non-bronchoscopic protected BAL was performed using a closed suction system with a 14 French 40 cm catheter inside to prevent aerosolization. After injection of 30-40 ml sterile normal saline into the endotracheal tube, the suction catheter was inserted through the endotracheal tube and blindly advanced into the distal airways till resistance was felt. The catheter was wedged. in that position and aspirate was collected in a sterile container into a sputum trap cup. Procedure was repeated if the aspirated fluid was less than 5 ml.

### U-PLEX Assays

U-PLEX Viral Combo 1 (human) kit which includes 20 analytes was purchased from Meso Scale Diagnostics (MSD, Rockville, Md.). The plate was read with a MESO QuickPlex SQ 120 imager and analyzed using Discovery Workbench v4.0 software. The assay was performed according to the manufacturer's instructions.

### TNF-α and IL-6 Quantification

Plasma concentrations of TNFα and IL-6 were measured using enzyme-linked immunosorbent assay (ELISA) kits (BioLegend, San Diego, Calif.). The operating procedure provided by the manufacturer was followed. One-hundred μL of plasma was used for each sample. The optical density (OD) at 450 nm was measured using a Synergy™ HT microplate reader (BioTek, Winooski, Vt.). Concentrations of TNF-α and IL-6 were determined using the standard curves. A few OD readings fell outside of the range of the standard curve, in which case a line of best fit was used to extrapolate the data.

### Ex Vivo Neutrophil Stimulation

Whole blood (1.50 uL) was lysed with ACK buffer. One-million cells were seeded in a 24-well plate and cultured with Brefeldin A solution for 20 min at 37° C. Cells were then stimulated with 250 ng/mL of LPS for 10 hours at 37° C. Following stimulation, cells were collected and washed with PBS prior to cell surface staining with Viability Dye-APC-Cy7, CD45-PE-Cy7, CD66b-PE, CD16-APC for 30 min at 4° C. Cells were washed again with PBS before fixation (Biolegend intracellular Fixation Buffer) for 30 min at RT. Cells were washed twice with permeabilization buffer (Biolegend Per Wash Buffer). Cells were incubated with TNFα-PerCP-Cy5.5 and IL-6-FITC overnight prior to washing and analysis on BD FACSCanto.

### Statistical Analysis

The two-tailed, unpaired Student t-test was used to determine the significance of differences between two groups. One-way ANOVA was used to determine differences between multiple groups. Since we have varied number of observations for each patient, we applied linear mixed effect models along with the Wald test statistics to compare the group differences, where group was considered as fixed effects, and patients were considered random effects. To examine association between two variables, we estimated the marginal Pearson correlation coefficient and tested its significance. The marginal Pearson correlation coefficient captures the association between two variables at the population level. The analyses were carded out in the Statistical software R (https://www.r-project.org/) and Prism version 10. A statistical test was claimed significant if p<0.05.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating coronavirus disease 2019 (COVID-19) in a subject, comprising the step of administering to the subject a therapeutically effective therapeutic agent,
(a) wherein the therapeutic agent inhibits CD66b⁺CD16^{Int}CD11b^{Int}CD44^{low}CD40⁺ low-density inflammatory band (LDIB) neutrophil population, or
(b) wherein the therapeutic agent inhibits COVID-19-associated coagulopathy (CAC).

2. (canceled)

3. A method of treating coronavirus disease 2019 (COVID-19) in a subject, comprising the step of administering to the subject a therapeutically effective therapeutic agent, wherein the subject has a lower level of CD16^{Int}CD44^{Low}CD11b^{Int} low-density neutrophils, and wherein the therapeutic agent is respiratory therapy.

4. A method of claim 3, wherein at a second time point as compared to a first time point, the respiratory therapy use is ceased.

5. A method of treating a subject having been diagnosed with coronavirus disease 2019 (COVID-19) with a therapeutic agent that inhibits low-density inflammatory neutrophil (LDN) population expressing intermediate levels of CD16 (CD16^{Int}).

6. The method of claim 5, wherein the LDN are CD66b⁺ LDN.

7. The method of claim 1, wherein the subject has elevated plasma levels of IL-10, IL-1RA, MCP-1 and/or MIP-1α as compared to a control.

8. The method of claim 1, wherein the subject has an elevated plasma level of IL-6 and/or TNF-α as compared to a control.

9. The method of claim 1, wherein the subject has an elevated plasma level of D-dimer as compared to a control.

10. The method of claim 1, wherein the subject has an elevated plasma level of ferritin as compared to a control.

11. The method of claim 1, wherein the subject has an elevated plasma level of D-dimer and ferritin.

12. The method of claim 1, wherein the subject is treated with a cytokine blocking antibody.

13. The method of claim 12, wherein the cytokine blocking antibody is tocilizumab, adalimumab, or etanercept.

14. The method of claim 1, wherein the subject is treated with an immunosuppressive regimen.

15. The method of claim 14, wherein the subject is treated with dexamethasone or anti-IL-6 therapy.

16. A method of detecting the severity level of coronavirus disease 2019 (COVID-19) in a subject, comprising measuring the level of CD16^{Int} low-density inflammatory neutrophil (LDN) in plasma as compared to a control.
